SEQUENCE LISTING (20 sequences)

SEQ ID NO: 1 - DNA, 21 bp, Artificial Sequence
  Description of Artificial Sequence Synthetic primer
ggatgtgtcgagtagtagggt

SEQ ID NO: 2 - DNA, 23 bp, Artificial Sequence
  Description of Artificial Sequence Synthetic primer
cctatcatctccacactaacgct

SEQ ID NO: 3 - DNA, 22 bp, Artificial Sequence
  Description of Artificial Sequence Synthetic primer
tgttaggggtgatttttagagg

SEQ ID NO: 4 - DNA, 29 bp, Artificial Sequence
  Description of Artificial Sequence Synthetic primer
attaaccccatttccatcctaactatctt

SEQ ID NO: 5 - DNA, 24 bp, Artificial Sequence
  Description of Artificial Sequence Synthetic primer
ttttagaggagtttttttttttta

SEQ ID NO: 6 - DNA, 22 bp, Artificial Sequence
  Description of Artificial Sequence Synthetic primer
gtgatyttggtyaytgtaayyt

SEQ ID NO: 7 - DNA, 28 bp, Artificial Sequence
  Description of Artificial Sequence Synthetic primer
tctcatctttccataracatttatttar

SEQ ID NO: 8 - DNA, 27 bp, Artificial Sequence
  Description of Artificial Sequence Synthetic primer
agggtttyaytatattagyyaggttgg

SEQ ID NO: 9 - DNA, 24 bp, Artificial Sequence
  Description of Artificial Sequence Synthetic primer
atrcaaaccatrcarcccttttrc

SEQ ID NO: 10 - DNA, 25 bp, Artificial Sequence
  Description of Artificial Sequence Synthetic primer
yyyaaaataytgagattataggtgt

SEQ ID NO: 11 - DNA, 23 bp, Artificial Sequence
  Description of Artificial Sequence Synthetic primer
taggagaaagggtttattgtggt

SEQ ID NO: 12 - DNA, 24 bp, Artificial Sequence
  Description of Artificial Sequence Synthetic primer
acacaccctacctttttcactcca

SEQ ID NO: 13 - DNA, 29 bp, Artificial Sequence
  Description of Artificial Sequence Synthetic primer
ggtattgatttatggttagggatttatag

SEQ ID NO: 14 - DNA, 27 bp, Artificial Sequence
  Description of Artificial Sequence Synthetic primer
aaacaaaaaaaactccacctccaatcc

SEQ ID NO: 15 - DNA, 21 bp, Artificial Sequence
  Description of Artificial Sequence Synthetic primer
gggatttatagttttgtgaga

SEQ ID NO: 16 - DNA, 32 bp, Artificial Sequence
  Description of Artificial Sequence Synthetic primer
agtyatgtyttytgyaaataaaaatggayayy

SEQ ID NO: 17 - DNA, 25 bp, Artificial Sequence
  Description of Artificial Sequence Synthetic primer
ttrctttttaaccaccaacactacc

SEQ ID NO: 18 - DNA, 29 bp, Artificial Sequence
  Description of Artificial Sequence Synthetic primer
yygtttytttyatyttytggtgatgttaa

SEQ ID NO: 19 - DNA, 30 bp, Artificial Sequence
  Description of Artificial Sequence Synthetic primer
ararrrcaatctctrrrtaatccacatrtr

SEQ ID NO: 20 - DNA, 22 bp, Artificial Sequence
  Description of Artificial Sequence Synthetic primer
ggtgatgttaatyttytgttta

(19) United States
(12) Patent Application Publication
(10) Pub. No.: US 2022/0267862 A1
(43) Pub. Date: Aug. 25, 2022

(54) DNA METHYLATION SIGNATURES OF CANCER IN HOST PERIPHERAL BLOOD MONONUCLEAR CELLS AND T CELLS

(71) Applicants: Moshe SZYF, Quebec (CA); Beijing Youan Hospital, Captical Medical University, Beijing (CN)

(72) Inventors: Moshe SZYF, Quebec (CA); Ning LI, Beijing (CN); Yonghong ZHANG, Beijing (CN); Sophie PETROPOULOS, Markham (CA)

(21) Appl. No.: 17/700,702

(22) Filed: Mar. 22, 2022

Related U.S. Application Data
(63) Continuation-in-part of application No. 16/309,322, filed on Dec. 12, 2018.

(51) Int. Cl. C12Q 1/6886

(52) U.S. Cl. CPC C12Q 1/6886; C12Q 2600/154; C12Q 2600/112

(57) ABSTRACT

A cancer has a DNA methylation signature in host T cells and Peripheral Blood Mononuclear Cells (PBMC) DNA. The present disclosure provides CG IDs derived from PBMC DNA for predicting hepatocellular carcinoma (HCC) stages and chronic hepatitis. Also disclosed are kits for predicting HCC using identified CG IDs and pyrosequencing DNA methylation assays, receiver operating characteristics (ROC) assays, penalized regression assays and hierarchical clustering analysis assays. The present disclosure provides DNA methylation signatures (CG IDs) that can be used for diagnosis, prognosis, and treatment of a cancer.

### CROSS-REFERENCE TO RELATED APPLICATIONS

This application is a continuation-in-part of U.S. application Ser. No. 16/309,322, filed on Dec. 12, 2019, which is a 371 U.S. National Phase of PCT International Application No. PCT/CN2016/086845, filed on Jun. 23, 2016, the entire content of each application listed above is incorporated by reference herein.

### SEQUENCE LISTING

The instant application contains a Sequence Listing which has been filed electronically in ASCII format and is hereby incorporated by reference in its entirety. Said ASCII copy, created on Jun. 18, 2019, is named 942301-1040_SL.txt and is 4,897 bytes in size.

### FIELD OF THE DISCLOSURE

The present disclosure relates to DNA methylation signatures in human DNA, particularly in the field of molecular diagnostics.

### BACKGROUND

Hepatocellular Carcinoma (HCC) is the fifth most common cancer world-wide (1). It is particularly prevalent in Asia, and its occurrence is highest in areas where hepatitis B is prevalent, indicating a possible causal relationship (2). Follow up of high-risk populations such as chronic hepatitis patients and early diagnosis of transitions from chronic hepatitis to HCC would improve cure rates. The survival rate of hepatocellular carcinoma is currently extremely low because it is almost always diagnosed at the late stages. Liver cancer could be effectively treated with cure rates of >80% if diagnosed early^{l}. Advances in imaging have improved noninvasive detection of HCC (3, 4). However, current diagnostic methods, which include imaging and immunoassays with single proteins such as alpha-fetoprotein often fail to diagnose HCC early (2). These challenges are not limited to HCC but common to other cancers as well. Molecular diagnosis of cancer is focused on tumors and biomaterial originating in tumor including tumor DNA in plasma (5, 6), circulating tumor cells (7) and the tumor-host microenvironment (8, 9). The prevailing and widely accepted hypothesis is that molecular changes that drive cancer initiation and progression originate primarily in the tumor itself and that relevant changes in the host occur primarily in the tumor microenvironment. The identity of immune cells in the tumor microenvironment has attracted therefore significant attention (10, 11).

DNA methylation, a covalent modification of DNA, which is a primary mechanism of epigenetic regulation of genome function is ubiquitously altered in tumors (12-15) including HCC (16). DNA methylation profiles of tumors distinguish different stages of tumor progression and are potentially robust tools for tumor classification, prognosis and prediction of response to chemotherapy (17). The major drawback for using tumor DNA methylation in early diagnosis is that it requires invasive procedures and anatomical visualization of the suspected tumor. Circulating tumor cells are a noninvasive source of tumor DNA and are used for measuring DNA methylation in tumor suppressor genes (18). Hypomethylation of HCC DNA is detectable in patients' blood (19) and genome wide bisulfite sequencing was recently applied to detect hypomethylated DNA in plasma from HCC patients (20). However, this source is limited, particularly at early stages of cancer and the DNA methylation profiles are confounded by host DNA methylation profiles.

The idea that host immuno-surveillance plays an important role in tumorigenesis by eliminating tumor cells and suppressing tumor growth has been proposed by Paul Ehrlich (21, 22) more than a century ago and has fallen out of favor since. However, accumulating data from both animal and human clinical studies suggest that the host immune system plays an important role in tumorigenesis through “immuno-editing” which involves three stages: elimination, equilibrium and escape (23-25). Presence of tumor infiltrating cytotoxic CD8+T cells associated with better prognosis in several clinical studies of human regressive melanoma (26-31), esophageal (32), ovarian (33, 34), and colorectal cancer (35-37). The immune system is believed to be responsible for the phenomenon of cancer dormancy when circulating cancer cells are detectable in the absence of clinical symptoms (15, 38). Interestingly, recent DNA methylation and transcriptome analysis of tumors revealed tumor stage specific immune signatures of infiltrating lymphocytes (39, 40). However, these signatures represent targeted immune cells in the tumor microenvironment and utilization of such signatures for early diagnosis requires invasive procedures. The tumor-infiltrating immune cells represent only a minor fraction of peripheral blood cells (41-44). Global DNA methylation changes were previously reported in leukocytes and EWAS studies revealed differences in DNA methylation in leukocytes from bladder, head and neck and ovarian cancer and these differences were independent of differences in white blood cell distribution (45). These studies were mainly aimed at identifying underlying DNA methylation changes in cancer genes that might serve as surrogate markers for changes in DNA methylation in the tumor. However, the question of whether the peripheral host immune system exhibits a distinct DNA methylation response to the cancer state that correlates with cancer progression has not been addressed.

### SUMMARY

The present disclosure provides that cancer progression is associated with distinct DNA methylation profiles in the host peripheral immune cells. These DNA methylation markers differentiate between cancer and the underlying chronic inflammatory liver disease are provided herein.

In certain embodiments, the present disclosure illustrate these DNA methylation profiles in a discovery set of 69 people from the Beijing area of China (10 controls and 10 patients for each of the following groups Hepatitis B, C, stages 1-3, and 9 patients for stage 4) of HCC staged using the EASL-EORTC Clinical Practice Guidelines for HCC (Table 1). In the present disclosure, a whole genome approach (Illumina 450k arrays) was used to delineate DNA methylation profiles without preconceived bias on the type of genes that might be involved. The disclosure method demonstrates for the first time specific DNA methylation profiles of Hepatitis B and C that are distinct from HCC as well as DNA methylation profiles for each of the different stages of HCC in peripheral blood mononuclear cells. These profiles do not show a significant overlap with the DNA methylation profiles of HCC tumors that have been previously described (16), suggesting that they reflect changes in peripheral blood mononuclear cells genomic functions and are not surrogates of changes in tumor DNA methylation. Thus, the present disclosure provides the DNA methylation changes in the host immune system in cancer. The present disclosure also provides a DNA methylation signature in host T cells in people suffering from cancer. The present disclosure further provides that there is a significant overlap between DNA methylation profiles delineated in PBMCs and T cells.

In certain embodiments, the present disclosure provides a validation of four (4) genes that were differentially methylated in T cells from HCC patients in the discovery cohort by pyrosequencing of T cells DNA in a separate cohort of patients (n=79).

The present disclosure further provides the utility of the disclosed diagnostic method in predicting cancer and stage of cancer of unknown samples using statistical models based on these DNA methylation signatures. The diagnostic methods disclosed herein provide important implications for understanding of the mechanisms of the disease and its treatment and provides noninvasive diagnostics of cancer in peripheral blood mononuclear cells DNA. Such diagnostic methods could be used by any person skilled in the art to derive DNA methylation signatures in the immune system of any cancer using any method for genome wide methylation mapping that are available to those skilled in the art such as for example genome wide bisulfite sequencing, capture sequencing, methylated DNA Immunoprecipitation (MeDIP) sequencing and any other method of genome wide methylation mapping that becomes available.

Preferred embodiments are provided as follows.

In the first aspect, the present disclosure provides DNA methylation signature of cancer in peripheral blood mononuclear cells (PBMC) for predicting cancer, said DNA methylation signature is derived using genome wide DNA methylation mapping methods, such as Illumina 450K or 850K arrays, genome wide bisulfite sequencing, methylated DNA Immunoprecipitation (MeDIP) sequencing or hybridization with oligonucleotide arrays.

In one embodiment, the DNA methylation signature is CG IDs derived from PBMC DNA listed below for predicting hepatocellular carcinoma (HCC) stages and chronic hepatitis using either

PBMC or T cells DNA methylation levels of said CG IDs.

| | | | | | |
|---|---|---|---|---|---|
| cg05375333 | cg24304617 | cg08649216 | cg15775914 | cg06098530 | cg04536922 |
| cg23679141 | cg26009832 | cg06908855 | cg21585138 | cg15514380 | cg20838429 |
| cg01546046 | cg27090007 | cg11412036 | cg00744866 | cg19988492 | cg21542922 |
| cg10036013 | cg24958366 | cg23824801 | cg08306955 | cg00361155 | cg11356004 |
| cg12829666 | cg17479131 | cg27408285 | cg15009198 | cg05423018 | cg19140262 |
| cg15011899 | cg27644327 | cg01810593 | cg18878210 | cg13710613 | cg05033369 |
| cg02001279 | cg11031737 | cg19795616 | cg02717454 | cg07072643 | cg09048334 |
| cg15188939 | cg09800500 | cg27284331 | cg22344162 | cg04018625 | cg04385818 |
| cg23311108 | cg02313495 | cg08575688 | cg26923863 | cg01238991 | cg01214050 |
| cg09789584 | cg16324306 | cg05486191 | cg15447825 | cg17741339 | cg14361741 |
| cg22301128 | cg02914652 | cg04171808 | cg04771084 | cg18132851 | cg16292016 |
| cg11737318 | cg11057824 | cg14276584 | cg23981150 | cg02556954 | cg14783904 |
| cg07118376 | cg26407558 | cg03496780 | cg24383056 | cg01359822 | cg26250154 |
| cg13978347 | cg09451574 | cg14375111 | cg24232444 | cg22747380 | cg02758552 |
| cg23544996 | cg21156970 | cg08944236 | cg22281935 | cg00211609 | cg21811450 |
| cg16306870 | cg01732538 | cg02142483 | cg22110158 | cg11911769 | cg03432151 |
| cg03731740 | cg10312296 | cg23102014 | cg04398282 | cg15755348 | cg08455089 |
| cg02749789 | cg17704839 | cg25683268 | cg08946713 | cg25195795 | cg17766305 |
| cg08123444 | cg24742520 | cg20460227 | cg24056269 | cg06151145 | cg06349546 |
| cg15747825 | cg14983135 | cg17163729 | cg15118835 | cg00568910 | cg23017594 |
| cg23829949 | cg21164050 | cg01417062 | cg14189441 | cg15146122 | cg12813441 |
| cg16712679 | cg06879746 | cg13146484 | cg16111924 | cg13615971 | cg01411912 |
| cg12820627 | cg27057509 | cg18417954 | cg27089675 | cg06194421 | cg15374754 |
| cg17534034 | cg23857976 | cg13913085 | cg07128102 | cg01966878 | cg00093544 |
| cg05591270 | cg05228338 | cg12705693 | cg18556587 | cg16565409 | cg14711743 |
| cg13219008 | cg24783785 | cg21579239 | cg02863594 | cg03044573 | cg00483304 |
| cg15607708 | cg27457290 | cg10274682 | cg08577341 | cg10469659 | cg24376286 |
| cg22475353 | cg14199837 | cg19389852 | cg12306086 | cg16240816 | cg27638509 |
| cg27296330 | cg25104397 | cg01839860 | cg21700582 | cg21487856 | cg11300809 |
| cg24449629 | cg20592700 | cg20222519 | cg14774438 | cg23486701 | cg09244071 |
| cg12177922 | cg27010159 | cg02272851 | cg15123819 | cg24640156 | cg00014638 |
| cg23004466 | cg14898127 | cg14734614 | cg00759807 | cg05086021 | cg00697672 |
| cg01696603 | cg11783497 | cg27120934 | cg07929642 | cg03899643 | cg01116137 |
| cg03639671 | cg08861115 | cg10078703 | cg08134863 | cg11556164 | cg20250700 |
| cg10203922 | cg15966610 | cg05099186 | cg20228731 | cg25135755 | cg15867698 |
| cg13749822 | cg13299325 | cg11767757 | cg23493018 | cg08113187 | cg11151251 |
| cg12263794 | cg22547775 | cg09545443 | cg04071270 | cg27588356 | cg05577016 |
| cg23157190 | cg22945413 | cg20427318 | cg20750319 | cg01611777 | cg01933228 |
| cg21406217 | cg15046123 | cg01698579 | cg12050434 | cg12299554 | cg11006453 |
| cg08247053 | cg26405097 | cg12691488 | cg00458932 | cg14356440 | cg03555836 |
| cg26576206 | cg03483626 | cg08568561 | cg25708982 | cg18482303 | cg02482718 |
| cg07212747 | cg14531436 | cg13943141 | cg12592365 | cg15323084 | cg24065504 |
| cg22872033 | cg20587236 | cg13619522 | cg19780570 | cg22876402 | cg09340198 |
| cg27186013 | cg24284882 | cg05502766 | cg20187173 | cg17092349 | cg22143698 |
| cg19851487 | cg17226602 | cg06445016 | cg07772781 | cg02782634 | cg07065759 |
| cg03481488 | cg22707529 | cg10895875 | cg01828328 | cg09987993 | cg21751540 |
| cg12598524 | cg19945957 | cg08634082 | cg05725404 | cg26401541 | cg20956548 |
| cg10761639 | cg05460226 | cg20944521 | cg14426660 | cg00248242 | cg18731803 |
| cg00350932 | cg25364972 | cg03252499 | cg04998202 | cg09514545 | cg09639931 |
| cg14914552 | cg00754989 | cg14762436 | cg07381872 | cg16476382 | cg16810031 |
| cg07504763 | cg01994308 | cg19266387 | cg14193653 | cg00189276 | cg10861953 |
| cg25279586 | cg23837109 | cg17934470 | cg22675447 | cg08858441 | cg12628061 |
| cg12019814 | cg10892950 | cg00758915 | cg09479286 | cg20874210 | cg06874640 |
| cg05941376 | cg02976588 | cg27143049 | cg00426720 | cg00321614 | cg15006843 |
| cg23044884 | cg24576298 | cg23880736 | cg05999692 | cg08226047 | cg25522867 |
| cg15891076 | cg12344600 | cg04090347 | cg10784548 | cg02265379 | cg01124132 |
| cg07145988 | cg27544294 | cg22515654 | cg12201380 | cg19925215 | cg10536529 |
| cg09635768 | cg00448395 | cg03062944 | cg05961707 | cg10995381 | cg16517298 |
| cg01124132 | cg10536529 | cg16517298 | cg18882449 | cg03909800 | cg18882449 |
| cg03909800 | | | | | |

In one embodiment, the DNA methylation signature is CG IDs derived from T cells listed below for predicting HCC stages and chronic hepatitis using PBMC or T cells DNA methylation levels of said CG IDs.

| | | | | | |
|---|---|---|---|---|---|
| cg00014638 | cg02015053 | cg03568507 | cg06098530 | cg08313420 | cg10918327 |
| cg00052964 | cg02086310 | cg03692651 | cg06168204 | cg08479516 | cg10923662 |
| cg00167275 | cg02132714 | cg03764364 | cg06279274 | cg08566455 | cg11065621 |
| cg00168785 | cg02142483 | cg03853208 | cg06445016 | cg08641990 | cg11080540 |
| cg00257775 | cg02152108 | cg03894796 | cg06477663 | cg08644463 | cg11157127 |
| cg00399683 | cg02193146 | cg03909800 | cg06488150 | cg08826152 | cg11231949 |
| cg00404641 | cg02314201 | cg03911306 | cg06568880 | cg08946713 | cg11262262 |
| cg00431894 | cg02322400 | cg03942932 | cg06652329 | cg09122035 | cg11556164 |
| cg00434461 | cg02490460 | cg03976645 | cg06816239 | cg09259081 | cg11692124 |
| cg00452133 | cg02536838 | cg04083575 | cg06822816 | cg09324669 | cg11706775 |
| cg00500229 | cg02556954 | cg04116354 | cg06850005 | cg09555124 | cg11718162 |
| cg00674365 | cg02710015 | cg04192168 | cg06895913 | cg09639931 | cg11909467 |
| cg00772991 | cg02717454 | cg04398282 | cg07019386 | cg09681977 | cg11955727 |
| cg00804338 | cg02750262 | cg04536922 | cg07052063 | cg09696535 | cg11958644 |
| cg00815832 | cg02849693 | cg04656070 | cg07065759 | cg09750084 | cg12019814 |
| cg00898013 | cg02863594 | cg04771084 | cg07145988 | cg10036013 | cg12099423 |
| cg01044293 | cg02914652 | cg04864807 | cg07249730 | cg10061361 | cg12161228 |
| cg01116137 | cg02939781 | cg04998202 | cg07266910 | cg10091662 | cg12299554 |
| cg01124132 | cg02976588 | cg05084827 | cg07381872 | cg10167378 | cg12315391 |
| cg01254303 | cg02991085 | cg05107535 | cg07385778 | cg10184328 | cg12427303 |
| cg01305421 | cg03035849 | cg05132077 | cg07721852 | cg10185424 | cg12549858 |
| cg01359822 | cg03151810 | cg05157625 | cg07772781 | cg10196532 | cg12583076 |
| cg01366985 | cg03204322 | cg05217983 | cg07834396 | cg10274682 | cg12649038 |
| cg01405107 | cg03215181 | cg05304366 | cg07850527 | cg10341310 | cg12691488 |
| cg01413790 | cg03400131 | cg05348875 | cg07912766 | cg10530883 | cg12727605 |
| cg01557792 | cg03441844 | cg05429448 | cg08038033 | cg10549831 | cg12777448 |
| cg01832672 | cg03461110 | cg05460226 | cg08113187 | cg10555744 | cg12789173 |
| cg01921773 | cg03541331 | cg05512157 | cg08123444 | cg10584024 | cg12856392 |
| cg01927745 | cg03544320 | cg05554346 | cg08280368 | cg10890302 | cg12868738 |
| cg01992590 | cg03546163 | cg05759347 | cg08306955 | cg10909506 | cg12880685 |
| cg12906381 | cg15009198 | cg17335387 | cg19795616 | cg22404498 | cg24919348 |
| cg12963656 | cg15011899 | cg17372657 | cg19841369 | cg22589728 | cg25100962 |
| cg12970155 | cg15046123 | cg17597631 | cg19930116 | cg22656550 | cg25104397 |
| cg13260278 | cg15109018 | cg17718703 | cg19988492 | cg22668906 | cg25174412 |
| cg13286116 | cg15145341 | cg17741339 | cg20197130 | cg22675447 | cg25188006 |
| cg13308137 | cg15302376 | cg17765025 | cg20222519 | cg22747380 | cg25310233 |
| cg13401703 | cg15331834 | cg17766305 | cg20478129 | cg22945413 | cg25353287 |
| cg13404054 | cg15514380 | cg17775490 | cg20585841 | cg23299919 | cg25459280 |
| cg13405775 | cg15514896 | cg17786894 | cg20587236 | cg23486701 | cg25461186 |
| cg13435137 | cg15598244 | cg17837517 | cg20606062 | cg23771949 | cg25502144 |
| cg13466988 | cg15695738 | cg17988310 | cg20625523 | cg23824902 | cg25673720 |
| cg13679714 | cg15704219 | cg18031596 | cg20769177 | cg23829949 | cg25779483 |
| cg13896699 | cg15720112 | cg18051353 | cg20781967 | cg23880736 | cg25784220 |
| cg13904970 | cg15747825 | cg18128914 | cg20995304 | cg23944804 | cg25891647 |
| cg13912027 | cg15756407 | cg18132851 | cg21092324 | cg24056269 | cg25964728 |
| cg13939291 | cg15867698 | cg18182216 | cg21222426 | cg24065504 | cg26015683 |
| cg14140403 | cg16111924 | cg18214661 | cg21226442 | cg24070198 | cg26250154 |
| cg14242995 | cg16218221 | cg18273840 | cg21358380 | cg24142603 | cg26325335 |
| cg14276584 | cg16259904 | cg18297196 | cg21384492 | cg24169486 | cg26402555 |
| cg14326196 | cg16292016 | cg18370682 | cg21386573 | cg24232444 | cg26405097 |
| cg14362178 | cg16306870 | cg18417954 | cg21487856 | cg24383056 | cg26407558 |
| cg14376836 | cg16496269 | cg18766900 | cg21816330 | cg24405716 | cg26465602 |
| cg14419424 | cg16512390 | cg18804667 | cg21833076 | cg24453118 | cg26475911 |
| cg14734614 | cg16763089 | cg18808261 | cg21918548 | cg24536818 | cg26594335 |
| cg14762436 | cg16810031 | cg19095568 | cg22088248 | cg24616553 | cg26803268 |
| cg14774438 | cg16894855 | cg19140262 | cg22143698 | cg24631428 | cg26827373 |
| cg14858267 | cg16924102 | cg19193595 | cg22256433 | cg24680439 | cg26856443 |
| cg14898127 | cg17144149 | cg19266387 | cg22301128 | cg24716416 | cg26876834 |
| cg14914552 | cg17173975 | cg19760965 | cg22303909 | cg24729928 | cg26963367 |
| cg15000827 | cg17221813 | cg19768229 | cg22374742 | cg24742520 | cg27010159 |
| cg27098685 | cg27113419 | cg27186013 | cg27207470 | cg27247736 | cg27300829 |
| cg27406664 | cg27408285 | cg27544294 | cg27576694 | | |

In one embodiment, the DNA methylation signature is CG IDs listed below for predicting different stages of HCC using DNA methylation measurements of said CG IDs in T cells or PBMC obtained by using statistical models such as penalized regression or clustering analysis.

Target CG IDs for separating HCC stage 1 from controls: cg14983135, cg10203922, cg05941376, cg14762436, cg12019814, cg14426660, cg18882449, cg02914652;

Target CG IDs for separating HCC stage 2 from controls: cg05941376, cg15188939, cg12344600, cg03496780, cg12019814;

Target CG IDs for separating HCC stage 3 from controls: cg05941376, cg02782634, cg27284331, cg12019814, cg23981150;

Target CG IDs for separating HCC stage 4 from controls: cg02782634, cg05941376, cg10203922, cg12019814, cg14914552, cg21164050, cg23981150;

Target CG IDs for separating HCC stage 1 from hepatitis B: cg05941376, cg10203922, cg11767757, cg04398282, cg11151251, cg24742520, cg14711743;

Target CG IDs for separating HCC stage 1 from stage 2-4: cg03252499, cg03481488, cg04398282, cg10203922, cg11783497, cg13710613, cg14762436, cg23486701;

Target CG IDs for separating HCC stage 2 from stage 3-4: cg02914652, cg03252499, cg11783497, cg11911769, cg12019814, cg14711743, cg15607708, cg20956548, cg22876402, cg24958366;

Target CG IDs for separating HCC stage 1-3 from stage 4: cg02782634, cg11151251, cg24958366, cg06874640, cg27284331, cg16476382, cg14711743.

In one embodiment, the DNA methylation signature is CG IDs listed below for predicting stages of HCC using DNA methylation measurements of said CG IDs in T cells or PBMC obtained by using statistical models such as penalized regression or clustering analysis,

| | | | | |
|---|---|---|---|---|
| cg14983135 | cg10203922 | cg05941376 | cg14762436 | cg12019814 |
| cg03496780 | cg02782634 | cg27284331 | cg23981150 | cg14914552 |
| cg13710613 | cg23486701 | cg11911769 | cg14711743 | cg15607708 |
| cg14426660 | cg18882449 | cg02914652 | cg15188939 | cg12344600 |
| cg21164050 | cg03252499 | cg03481488 | cg04398282 | cg11783497 |
| cg20956548 | cg22876402 | cg24958366 | cg11151251 | cg06874640 |
| cg16476382 | | | | |

In the second aspect, the present disclosure provides a kit for predicting cancer, comprising means and reagents for detecting DNA methylation measurements of the DNA methylation signature.

In one embodiment, the present disclosure provides a kit for predicting hepatocellular carcinoma (HCC) stages and chronic hepatitis, comprising means and reagents for detecting DNA methylation measurements of the CG IDs of table 3 in embodiment.

In one embodiment, the present disclosure provides a kit for predicting HCC stages and chronic hepatitis, comprising means and reagents for detecting DNA methylation measurements of the CG IDs of table 6 in embodiment.

In one embodiment, the present disclosure provides a kit for predicting different stages of HCC, comprising means and reagents for detecting DNA methylation measurements of the CG IDs of table 4 in embodiment.

In one embodiment, the present disclosure provides a kit for predicting stages of HCC, comprising means and reagents for detecting DNA methylation measurements of the CG IDs of table 5 in embodiment.

In the third aspect, the present disclosure provides gene pathways that are epigenetically regulated in cancer in peripheral immune system.

In the fourth aspect, the present disclosure provides use of CG IDs disclosed herein

In one embodiment, present disclosure provides use of DNA pyrosequencing methylation assays for predicting HCC by using CG IDs listed above, for example using the below disclosed primers for:

AHNAK (outside forward; GGATGTGTCGAGTAGTAGGGT (SEQ ID NO:1), outside reverse CCTATCATCTCCACACTAACGCT (SEQ ID NO:2), nested forward TGTTAGGGGTGATTTTTAGAGG (SEQ ID NO:3), nested reverse ATTAACCCCATTTCCATCCTAACTATCTT (SEQ ID NO:4), and sequencing primer TTTTAGAGGAGTTTTTTTTTTTTA) (SEQ ID NO:5);

SLFN2L (outside forward GTGATYTTGGTYAYTGTAAYYT (SEQ ID NO:6), Outside reverse TCTCATCTTTCCATARACATTTATTTAR (SEQ ID NO:7), forward nested AGGGTTTYAYTATATTAGYYAGGTTGG (SEQ ID NO:8), reverse nested ATRCAAACCATRCARCCCTTTTRC (SEQ ID NO:9), sequencing primer YYYAAAATAYTGAGATTATAGGTGT (SEQ ID NO:10));

AKAP7 (outside forward TAGGAGAAAGGGTTTATTGTGGT (SEQ ID NO:11), outside reverse ACACACCCTACCTTTTTCACTCCA (SEQ ID NO:12), nested forward GGTATTGATTTATGGTTAGGGATTTATAG (SEQ ID NO:13), nested reverse AAACAAAAAAAACTCCACCTCCAATCC (SEQ ID NO:14), sequencing primer GGGATTTATAGTTTTGTGAGA (SEQ ID NO:15)); and

STAP1 (outside forward AGTYATGTYTTYTGYAAATAAAAATGGAYAYY (SEQ ID NO:16), outside reverse, TTRCTTTTTAACCACCAACACTACC (SEQ ID NO:17) nested forward YYGTTTYTTTYATYTTYTGGTGATGTTAA (SEQ ID NO:18), nested reverse ARARRRCAATCTCTRRRTAATCCACATRTR (SEQ ID NO:19), sequencing primer GGTGATGTTAATYTTYTGTTTA (SEQ ID NO:20)).

In one embodiment, present disclosure provides use of Receiver operating characteristics (ROC) assays for predicting HCC by using CG IDs listed above, for example STAP1 (cg04398282).

In one embodiment, present disclosure provides use of hierarchical Clustering analysis for predicting HCC by using CG IDs listed above.

In the fifth aspect, the present disclosure provides method for identifying DNA methylation signature for predicting disease, comprising the step of performing statistical analysis on DNA methylation measurements obtained from samples.

In one embodiment, the method comprises the step of performing statistical analysis on DNA methylation measurements obtained from samples, said DNA methylation measurements are obtained by performing Illumina Beadchip 450K or 850K assay of DNA extracted from sample.

In one embodiment, said DNA methylation measurements are obtained by performing DNA pyrosequencing, mass spectrometry based (Epityper™) or PCR based methylation assays of DNA extracted from sample.

In one embodiment, the method comprises the step of performing statistical analysis on DNA methylation measurements obtained from samples; said statistical analysis includes Pearson correlation.

In one embodiment, said statistical analysis includes Receiver operating characteristics (ROC) assays.

In one embodiment, said statistical analysis includes hierarchical clustering analysis assays.

### DEFINITIONS

As used herein, the term “CG” refers to a di-nucleotide sequence in DNA containing cytosine and guanosine bases. These di-nucleotide sequences could become methylated in human and other animal DNA. The CG ID reveals its position in the human genome as defined by the Illlumina 450K manifest (The annotation of the CGs listed herein is publicly available and installed as an R package IlluminaHumanMethylation450k.db as described in Triche T and Jr. IlluminaHumanMethylation450k.db: Illumina Human Methylation 450k annotation data. R package version 2.0.9.). Annotated CGs useful herein are provided below:

| CG ID | Chr | Start | End | Distance to TSS | Gene Name |
|---|---|---|---|---|---|
| cg00014638 | chr10 | 94113651 | 94113652 | 62732 | 5-Mar |
| cg00093544 | chr22 | 30901640 | 30901641 | 57 | SEC14L4 |
| cg00189276 | chr6 | 1410267 | 1410268 | −19622 | MIR6720 |
| cg00211609 | chr1 | 1178039 | 1178040 | 4062 | FAM132A |
| cg00248242 | chr2 | 240867059 | 240867060 | 15451 | MIR4786 |
| cg00321614 | chr5 | 172856932 | 172856933 | 82475 | MIR8056 |
| cg00350932 | chr2 | 86335912 | 86335913 | 2608 | PTCD3 |
| cg00361155 | chr2 | 109651951 | 109651952 | −46124 | EDAR |
| cg00426720 | chr2 | 157187204 | 157187205 | 2082 | NR4A2 |
| cg00448395 | chr7 | 124570359 | 124570360 | −323 | POT1 |
| cg00458932 | chr2 | 208199465 | 208199466 | −80426 | LOC101927865 |
| cg00483304 | chr2 | 64976962 | 64976963 | −95917 | SERTAD2 |
| cg00568910 | chr1 | 43429380 | 43429381 | −4534 | SLC2A1 |
| cg00697672 | chr16 | 89151343 | 89151344 | −8873 | ACSF3 |
| cg00744866 | chr3 | 33701209 | 33701210 | −277 | CLASP2 |
| cg00754989 | chr15 | 72530044 | 72530045 | −6318 | PKM |
| cg00758915 | chr5 | 140773539 | 140773540 | 2057 | PCDHGA8 |
| cg00759807 | chr16 | 89390789 | 89390790 | 3249 | LOC100287036 |
| cg01116137 | chr20 | 25034263 | 25034264 | 4554 | ACSS1 |
| cg01124132 | chr22 | 32599511 | 32599512 | −48 | RFPL2 |
| cg01214050 | chr16 | 54155639 | 54155640 | 82335 | FTO-IT1 |
| cg01238991 | chr8 | 99076314 | 99076315 | −435 | ERICH5 |
| cg01359822 | chr21 | 40176597 | 40176598 | −633 | ETS2 |
| cg01411912 | chr1 | 153517265 | 153517266 | 1016 | S100A4 |
| cg01417062 | chr10 | 63246532 | 63246533 | 6734 | TMEM26-AS1 |
| cg01546046 | chr14 | 31494849 | 31494850 | 174 | AP4S1 |
| cg01611777 | chr2 | 102359027 | 102359028 | 44490 | MAP4K4 |
| cg01696603 | chr6 | 125623444 | 125623445 | −163 | HDDC2 |
| cg01698579 | chr13 | 112823520 | 112823521 | −28126 | LINC01070 |
| cg01732538 | chr2 | 181738144 | 181738145 | −106967 | UBE2E3 |
| cg01810593 | chr8 | 22464288 | 22464289 | 1750 | CCAR2 |
| cg01828328 | chr8 | 134310946 | 134310947 | −1400 | NDRG1 |
| cg01839860 | chr5 | 138957422 | 138957423 | 16672 | UBE2D2 |
| cg01933228 | chr1 | 100316637 | 100316638 | 107 | AGL |
| cg01966878 | chr4 | 90757139 | 90757140 | −412 | SNCA-AS1 |
| cg01994308 | chr8 | 57122990 | 57122991 | 868 | PLAG1 |
| cg02001279 | chr19 | 940967 | 940968 | 14931 | ARID3A |
| cg02142483 | chr16 | 84560555 | 84560556 | −22268 | TLDC1 |
| cg02265379 | chr5 | 87898506 | 87898507 | 64250 | MIR9-2 |
| cg02272851 | chr10 | 3797471 | 3797472 | 30001 | KLF6 |
| cg02313495 | chr15 | 40398007 | 40398008 | 279 | BMF |
| cg02482718 | chr1 | 4726759 | 4726760 | 11655 | AJAP1 |
| cg02556954 | chr5 | 137848577 | 137848578 | 28822 | ETF1 |
| cg02717454 | chr16 | 3928799 | 3928800 | 1321 | CREBBP |
| cg02749789 | chr17 | 1303531 | 1303532 | 24 | YWHAE |
| cg02758552 | chr3 | 49395714 | 49395715 | 76 | GPX1 |
| cg02782634 | chr17 | 57916643 | 57916644 | −1983 | MIR21 |
| cg02863594 | chr6 | 33280199 | 33280200 | 1964 | TAPBP |
| cg02914652 | chr12 | 4417142 | 4417143 | −13216 | C12orf5 |
| cg02976588 | chr1 | 150135546 | 150135547 | 13377 | PLEKHO1 |
| cg03044573 | chr1 | 173835265 | 173835266 | −100 | SNORD44 |
| cg03062944 | chr10 | 6183455 | 6183456 | −3387 | PFKFB3 |
| cg03252499 | chr11 | 124324477 | 124324478 | −13497 | OR8B8 |
| cg03432151 | chr15 | 89745000 | 89745001 | 19921 | RLBP1 |
| cg03481488 | chr10 | 81091172 | 81091173 | −16047 | PPIF |
| cg03483626 | chr1 | 111218276 | 111218277 | −622 | KCNA3 |
| cg03496780 | chr7 | 92466842 | 92466843 | −902 | CDK6 |
| cg03555836 | chr8 | 41422764 | 41422765 | −12942 | AGPAT6 |
| cg03639671 | chr4 | 145430689 | 145430690 | −136458 | HHIP |
| cg03731740 | chr1 | 29062689 | 29062690 | −443 | YTHDF2 |
| cg03899643 | chr1 | 90205170 | 90205171 | −81402 | LRRC8D |
| cg03909800 | chr6 | 76458005 | 76458006 | −887 | MYO6 |
| cg04018625 | chr2 | 171608293 | 171608294 | −18898 | ERICH2 |
| cg04071270 | chr5 | 140457553 | 140457554 | 55 | LOC101926905 |
| cg04090347 | chr21 | 44061597 | 44061598 | −12264 | PDE9A |
| cg04171808 | chr11 | 35188437 | 35188438 | 28021 | CD44 |
| cg04385818 | chr19 | 49468626 | 49468627 | 61 | FTL |
| cg04398282 | chr4 | 68424256 | 68424257 | −189 | STAP1 |
| cg04536922 | chr4 | 89978566 | 89978567 | −221 | FAM13A |
| cg04771084 | chr6 | 31973255 | 31973256 | −103 | CYP21A2 |
| cg04998202 | chr1 | 61545546 | 61545547 | −1987 | NFIA |
| cg05033369 | chr1 | 161676469 | 161676470 | −292 | FCRLA |
| cg05086021 | chr6 | 28829253 | 28829254 | 2200 | LOC401242 |
| cg05099186 | chr13 | 39923838 | 39923839 | 253517 | LHFP |
| cg05228338 | chr1 | 150048339 | 150048340 | 8615 | VPS45 |
| cg05375333 | chr12 | 99549023 | 99549024 | −156 | ANKS1B |
| cg05423018 | chr7 | 36193854 | 36193855 | 1019 | EEPD1 |
| cg05460226 | chr17 | 8804279 | 8804280 | 11554 | PIK3R5 |
| cg05486191 | chr7 | 5937190 | 5937191 | −1150 | CCZ1 |
| cg05502766 | chr3 | 122604506 | 122604507 | −853 | LOC100129550 |
| cg05577016 | chr4 | 7945149 | 7945150 | −3497 | AFAP1 |
| cg05591270 | chr10 | 80732609 | 80732610 | 94595 | ZMIZ1-AS1 |
| cg05725404 | chr16 | 58534157 | 58534158 | 111 | NDRG4 |
| cg05941376 | chr5 | 167836834 | 167836835 | −76628 | RARS |
| cg05961707 | chr10 | 104881879 | 104881880 | 71183 | NT5C2 |
| cg05999692 | chr6 | 23414372 | 23414373 | −712041 | NRSN1 |
| cg06098530 | chr10 | 76727919 | 76727920 | 90352 | DUPD1 |
| cg06151145 | chr4 | 48346434 | 48346435 | 2822 | SLAIN2 |
| cg06194421 | chr17 | 33570128 | 33570129 | 43 | SLFN5 |
| cg06349546 | chr22 | 43011285 | 43011286 | 35 | RNU12 |
| cg06445016 | chr8 | 61835848 | 61835849 | 44458 | LOC100130298 |
| cg06874640 | chr6 | 12716655 | 12716656 | −381 | PHACTR1 |
| cg06879746 | chr6 | 30883768 | 30883769 | 1661 | VARS2 |
| cg06908855 | chr7 | 93201042 | 93201043 | 2999 | CALCR |
| cg07065759 | chr2 | 198017462 | 198017463 | 149780 | ANKRD44-IT1 |
| cg07072643 | chr19 | 14785593 | 14785594 | 136 | EMR3 |
| cg07118376 | chr8 | 62624872 | 62624873 | 2326 | ASPH |
| cg07128102 | chr16 | 84221332 | 84221333 | −657 | TAF1C |
| cg07145988 | chr1 | 8692312 | 8692313 | 185386 | RERE |
| cg07212747 | chr16 | 4539233 | 4539234 | −6586 | HMOX2 |
| cg07381872 | chr1 | 61408076 | 61408077 | 28371 | NFIA-AS2 |
| cg07504763 | chr1 | 198575077 | 198575078 | −33020 | PTPRC |
| cg07772781 | chr3 | 101798142 | 101798143 | 138440 | LOC152225 |
| cg07929642 | chr16 | 89390685 | 89390686 | 3145 | LOC100287036 |
| cg08113187 | chr16 | 87469329 | 87469330 | 43529 | MAP1LC3B |
| cg08123444 | chr2 | 9833101 | 9833102 | −61918 | YWHAQ |
| cg08134863 | chr16 | 89390968 | 89390969 | 3428 | LOC100287036 |
| cg08226047 | chr21 | 15144580 | 15144581 | 48071 | MIR8069-1 |
| cg08247053 | chr1 | 34175317 | 34175318 | −150758 | HMGB4 |
| cg08306955 | chr6 | 25137971 | 25137972 | 79 | CMAHP |
| cg08455089 | chr6 | 37292135 | 37292136 | −29612 | RNF8 |
| cg08568561 | chr7 | 42834498 | 42834499 | −88453 | LINC01448 |
| cg08575688 | chr2 | 228678500 | 228678501 | −57 | CCL20 |
| cg08577341 | chr5 | 167001123 | 167001124 | 289281 | TENM2 |
| cg08634082 | chr1 | 241801700 | 241801701 | 2000 | OPN3 |
| cg08649216 | chr7 | 135344844 | 135344845 | −2376 | C7orf73 |
| cg08858441 | chr1 | 569427 | 569428 | −1635 | MIR6723 |
| cg08861115 | chr2 | 113735377 | 113735378 | −218 | IL36G |
| cg08944236 | chr16 | 53242355 | 53242356 | 153411 | CHD9 |
| cg08946713 | chr2 | 191844998 | 191844999 | 33977 | STAT1 |
| cg09048334 | chr6 | 37012640 | 37012641 | 39218 | FGD2 |
| cg09244071 | chr7 | 101768746 | 101768747 | −159606 | SH2B2 |
| cg09340198 | chr3 | 15902540 | 15902541 | −1488 | ANKRD28 |
| cg09451574 | chr4 | 113069076 | 113069077 | 2524 | C4orf32 |
| cg09479286 | chr2 | 169659182 | 169659183 | 76 | NOSTRIN |
| cg09514545 | chr19 | 54200652 | 54200653 | −134 | MIR525 |
| cg09545443 | chr3 | 106960066 | 106960067 | 528 | LINC00883 |
| cg09635768 | chr1 | 8601318 | 8601319 | −117572 | RERE |
| cg09639931 | chr17 | 38024394 | 38024395 | −60 | ZPBP2 |
| cg09789584 | chr17 | 45144857 | 45144858 | −24779 | ARL17A |
| cg09800500 | chr12 | 24992256 | 24992257 | 63065 | BCAT1 |
| cg09987993 | chr2 | 69381969 | 69381970 | 51156 | MIR3126 |
| cg10036013 | chr7 | 4778839 | 4778840 | −36422 | AP5Z1 |
| cg10078703 | chr11 | 35963440 | 35963441 | −2171 | LDLRAD3 |
| cg10203922 | chr4 | 145566200 | 145566201 | −947 | HHIP |
| cg10274682 | chr19 | 6496041 | 6496042 | 6553 | TUBB4A |
| cg10312296 | chr16 | 34404524 | 34404525 | 237 | UBE2MP1 |
| cg10469659 | chr15 | 51057714 | 51057715 | 195 | SPPL2A |
| cg10536529 | chr2 | 105477284 | 105477285 | 5316 | POU3F3 |
| cg10761639 | chr1 | 2023794 | 2023795 | −12360 | PRKCZ |
| cg10784548 | chr5 | 176571350 | 176571351 | 10518 | NSD1 |
| cg10861953 | chr15 | 93892667 | 93892668 | −260225 | RGMA |
| cg10892950 | chr12 | 45626938 | 45626939 | −17150 | PLEKHA8P1 |
| cg10895875 | chr7 | 56242407 | 56242408 | −58318 | NUPR1L |
| cg10995381 | chr5 | 7877198 | 7877199 | 7982 | MTRR |
| cg11006453 | chr8 | 141599185 | 141599186 | 46460 | AGO2 |
| cg11031737 | chr11 | 27255755 | 27255756 | −14096 | BBOX1-AS1 |
| cg11057824 | chr14 | 50471938 | 50471939 | 2299 | C14orf182 |
| cg11151251 | chr14 | 69522003 | 69522004 | 75605 | ACTN1-AS1 |
| cg11300809 | chr2 | 223288637 | 223288638 | −684 | SGPP2 |
| cg11356004 | chr5 | 150948901 | 150948902 | −397 | FAT2 |
| cg11412036 | chr15 | 43941871 | 43941872 | −829 | CATSPER2 |
| cg11556164 | chr7 | 110738315 | 110738316 | 7254 | LRRN3 |
| cg11737318 | chr8 | 131440305 | 131440306 | 15600 | ASAP1 |
| cg11767757 | chr21 | 40145404 | 40145405 | −4 | LINC00114 |
| cg11783497 | chr2 | 113875292 | 113875293 | −177 | IL1RN |
| cg11911769 | chr7 | 101768676 | 101768677 | −159676 | SH2B2 |
| cg12019814 | chr8 | 117861247 | 117861248 | −25415 | RAD21-AS1 |
| cg12050434 | chr12 | 43030949 | 43030950 | 9350 | LOC101927058 |
| cg12177922 | chr1 | 154245232 | 154245233 | 194 | HAX1 |
| cg12201380 | chr10 | 123717181 | 123717182 | 17561 | NSMCE4A |
| cg12263794 | chr6 | 27791530 | 27791531 | −372 | HIST1H4J |
| cg12299554 | chr15 | 94840953 | 94840954 | −476 | MCTP2 |
| cg12306086 | chr4 | 106117747 | 106117748 | 49906 | TET2 |
| cg12344600 | chr6 | 89769123 | 89769124 | −21305 | PNRC1 |
| cg12592365 | chr17 | 78765948 | 78765949 | 13483 | LOC101928855 |
| cg12598524 | chr2 | 46088325 | 46088326 | 209283 | PRKCE |
| cg12628061 | chr1 | 56453730 | 56453731 | 591526 | PPAP2B |
| cg12691488 | chr1 | 243053673 | 243053674 | 211372 | LINC01347 |
| cg12705693 | chr5 | 912860 | 912861 | 19892 | TRIP13 |
| cg12813441 | chr2 | 55239331 | 55239332 | −1862 | RTN4 |
| cg12820627 | chr2 | 207147089 | 207147090 | 7338 | ZDBF2 |
| cg12829666 | chr3 | 153840379 | 153840380 | 1231 | ARHGEF26 |
| cg13146484 | chr14 | 61645461 | 61645462 | 103068 | TMEM30B |
| cg13219008 | chr19 | 9695776 | 9695777 | −568 | ZNF121 |
| cg13299325 | chr6 | 447777 | 447778 | 56039 | IRF4 |
| cg13615971 | chr15 | 92392821 | 92392822 | −4116 | SLCO3A1 |
| cg13619522 | chr15 | 75095171 | 75095172 | −10022 | LMAN1L |
| cg13710613 | chr9 | 140574551 | 140574552 | 61108 | EHMT1 |
| cg13749822 | chr4 | 145566663 | 145566664 | −484 | HHIP |
| cg13913085 | chr17 | 52996635 | 52996636 | 18584 | TOM1L1 |
| cg13943141 | chr9 | 93205862 | 93205863 | −10092 | LINC01508 |
| cg13978347 | chr9 | 120140243 | 120140244 | 37073 | ASTN2 |
| cg14189441 | chr10 | 30971547 | 30971548 | −9655 | SVILP1 |
| cg14193653 | chr9 | 94178868 | 94178869 | 7275 | NFIL3 |
| cg14199837 | chr1 | 151164109 | 151164110 | −1421 | VPS72 |
| cg14276584 | chr9 | 99318213 | 99318214 | 10989 | CDC14B |
| cg14356440 | chr2 | 135050894 | 135050895 | 39065 | MGAT5 |
| cg14361741 | chr9 | 71685390 | 71685391 | 34912 | FXN |
| cg14375111 | chr3 | 14165186 | 14165187 | 1184 | CHCHD4 |
| cg14426660 | chr10 | 5488500 | 5488501 | −13 | NET1 |
| cg14531436 | chr8 | 140928796 | 140928797 | −213498 | KCNK9 |
| cg14711743 | chr5 | 79514577 | 79514578 | 37320 | SERINC5 |
| cg14734614 | chr19 | 51473346 | 51473347 | −418 | KLK6 |
| cg14762436 | chr7 | 24917750 | 24917751 | 14489 | OSBPL3 |
| cg14774438 | chr11 | 111957396 | 111957397 | 125 | TIMM8B |
| cg14783904 | chr17 | 9729422 | 9729423 | 42 | GLP2R |
| cg14898127 | chr15 | 81587493 | 81587494 | −1760 | IL16 |
| cg14914552 | chr8 | 97340188 | 97340189 | 66075 | PTDSS1 |
| cg14983135 | chr7 | 48129822 | 48129823 | 972 | UPP1 |
| cg15006843 | chr1 | 205720633 | 205720634 | −1262 | NUCKS1 |
| cg15009198 | chr2 | 97429502 | 97429503 | 2864 | CNNM4 |
| cg15011899 | chr13 | 111854118 | 111854119 | 14946 | ARHGEF7 |
| cg15046123 | chr6 | 15421581 | 15421582 | 172496 | JARID2 |
| cg15118835 | chr5 | 75469826 | 75469827 | 90588 | SV2C |
| cg15123819 | chr5 | 99388688 | 99388689 | 335269 | LOC100133050 |
| cg15146122 | chr2 | 40472772 | 40472773 | 184671 | SLC8A1 |
| cg15188939 | chr15 | 72809154 | 72809155 | 42488 | ARIH1 |
| cg15323084 | chr1 | 1556707 | 1556708 | 5463 | MIB2 |
| cg15374754 | chr18 | 76696950 | 76696951 | −43324 | SALL3 |
| cg15447825 | chr13 | 113873353 | 113873354 | 9535 | CUL4A |
| cg15514380 | chr21 | 38737243 | 38737244 | −2615 | DYRK1A |
| cg15607708 | chr19 | 54041308 | 54041309 | −24 | ZNF331 |
| cg15747825 | chr6 | 28565626 | 28565627 | −10515 | ZBED9 |
| cg15755348 | chr7 | 101768874 | 101768875 | −159478 | SH2B2 |
| cg15775914 | chr1 | 241799084 | 241799085 | 147 | CHML |
| cg15867698 | chr14 | 69438267 | 69438268 | 7815 | ACTN1 |
| cg15891076 | chr10 | 65930618 | 65930619 | 649496 | REEP3 |
| cg15966610 | chr8 | 79718206 | 79718207 | −449 | IL7 |
| cg16111924 | chr7 | 138348981 | 138348982 | −13 | SVOPL |
| cg16240816 | chr2 | 65861662 | 65861663 | −202007 | SPRED2 |
| cg16292016 | chr5 | 42424356 | 42424357 | −197 | GHR |
| cg16306870 | chr3 | 194868790 | 194868791 | 191 | XXYLT1-AS2 |
| cg16324306 | chr14 | 93786330 | 93786331 | 13107 | BTBD7 |
| cg16476382 | chr2 | 189169831 | 189169832 | 7613 | MIR561 |
| cg16517298 | chr1 | 230413174 | 230413175 | 148499 | PGBD5 |
| cg16565409 | chr17 | 27048223 | 27048224 | 656 | SNORD42B |
| cg16712679 | chr12 | 42719762 | 42719763 | 169 | ZCRB1 |
| cg16810031 | chr17 | 38024146 | 38024147 | −308 | ZPBP2 |
| cg17092349 | chr3 | 49058272 | 49058273 | −131 | MIR191 |
| cg17163729 | chr2 | 554372 | 554373 | 123066 | TMEM18 |
| cg17226602 | chr5 | 154393494 | 154393495 | 235 | KIF4B |
| cg17479131 | chr7 | 149567078 | 149567079 | −2978 | ATP6V0E2 |
| cg17534034 | chr8 | 106586880 | 106586881 | 255734 | ZFPM2 |
| cg17704839 | chr19 | 9939038 | 9939039 | 471 | UBL5 |
| cg17741339 | chr6 | 152085619 | 152085620 | −41188 | ESR1 |
| cg17766305 | chr10 | 90147030 | 90147031 | 196051 | RNLS |
| cg17934470 | chr5 | 49959703 | 49959704 | −2029 | PARP8 |
| cg18132851 | chr6 | 152085641 | 152085642 | −41166 | ESR1 |
| cg18417954 | chr19 | 55672513 | 55672514 | −3414 | TNNI3 |
| cg18482303 | chr2 | 135041380 | 135041381 | 29551 | MGAT5 |
| cg18556587 | chr2 | 159909020 | 159909021 | 83875 | TANC1 |
| cg18731803 | chr19 | 9903129 | 9903130 | −23720 | ZNF846 |
| cg18878210 | chr10 | 77021880 | 77021881 | −26111 | COMTD1 |
| cg18882449 | chr10 | 104885122 | 104885123 | 67940 | NT5C2 |
| cg19140262 | chr6 | 99380488 | 99380489 | 15393 | FBXL4 |
| cg19266387 | chr3 | 183596123 | 183596124 | 6569 | PARL |
| cg19389852 | chr1 | 145439013 | 145439014 | 576 | TXNIP |
| cg19780570 | chr5 | 133764548 | 133764549 | 6189 | LOC102546229 |
| cg19795616 | chr7 | 106371890 | 106371891 | −70257 | CCDC71L |
| cg19851487 | chr19 | 49655517 | 49655518 | 3163 | HRC |
| cg19925215 | chr8 | 80964918 | 80964919 | −22413 | MRPS28 |
| cg19945957 | chr20 | 33264901 | 33264902 | 187 | PIGU |
| cg19988492 | chr21 | 38807712 | 38807713 | 15111 | DYRK1A |
| cg20187173 | chr3 | 177370839 | 177370840 | −163813 | LOC102724550 |
| cg20222519 | chr3 | 23245916 | 23245917 | 1133 | UBE2E2 |
| cg20228731 | chr7 | 130646051 | 130646052 | 47829 | LOC100506860 |
| cg20250700 | chr7 | 100251432 | 100251433 | 2651 | ACTL6B |
| cg20427318 | chr6 | 134757763 | 134757764 | −1090 | LINC01010 |
| cg20460227 | chr2 | 120452632 | 120452633 | 15890 | TMEM177 |
| cg20587236 | chr12 | 109900956 | 109900957 | 14198 | KCTD10 |
| cg20592700 | chr7 | 5230083 | 5230084 | 249 | WIPI2 |
| cg20750319 | chr7 | 625089 | 625090 | −17392 | LOC101926963 |
| cg20838429 | chr2 | 163100512 | 163100513 | −468 | FAP |
| cg20874210 | chr11 | 45716004 | 45716005 | −2679 | MIR7154 |
| cg20944521 | chr14 | 22218494 | 22218495 | 85198 | OR4E2 |
| cg20956548 | chr19 | 56618060 | 56618061 | 14681 | ZNF787 |
| cg21156970 | chr7 | 47711149 | 47711150 | 16308 | C7orf65 |
| cg21164050 | chr13 | 27757411 | 27757412 | 11016 | USP12-AS2 |
| cg21406217 | chr8 | 28748500 | 28748501 | 276 | HMBOX1 |
| cg21487856 | chr2 | 54828502 | 54828503 | 42972 | SPTBN1 |
| cg21542922 | chr4 | 187680768 | 187680769 | −35782 | FAT1 |
| cg21579239 | chr15 | 43211292 | 43211293 | 1714 | TTBK2 |
| cg21585138 | chr3 | 50645106 | 50645107 | 4155 | CISH |
| cg21700582 | chr7 | 93474119 | 93474120 | 46183 | TFPI2 |
| cg21751540 | chr19 | 21541537 | 21541538 | −197 | ZNF738 |
| cg21811450 | chr22 | 47022471 | 47022472 | −186 | GRAMD4 |
| cg22110158 | chr11 | 130036542 | 130036543 | 6861 | ST14 |
| cg22143698 | chr5 | 10608058 | 10608059 | 43624 | ANKRD33B |
| cg22281935 | chr2 | 162934111 | 162934112 | −3060 | DPP4 |
| cg22301128 | chr4 | 77011716 | 77011717 | 15869 | ART3 |
| cg22344162 | chr1 | 167523769 | 167523770 | −714 | CREG1 |
| cg22475353 | chr19 | 54041163 | 54041164 | −169 | ZNF331 |
| cg22515654 | chr1 | 10590672 | 10590673 | 55670 | PEX14 |
| cg22547775 | chr5 | 2537634 | 2537635 | 214134 | IRX2 |
| cg22675447 | chr1 | 24745395 | 24745396 | 3151 | NIPAL3 |
| cg22707529 | chr6 | 143999715 | 143999716 | 614 | PHACTR2 |
| cg22747380 | chr8 | 118993090 | 118993091 | 130967 | EXT1 |
| cg22872033 | chr14 | 21725703 | 21725704 | 11934 | HNRNPC |
| cg22876402 | chr3 | 71553543 | 71553544 | 37696 | MIR1284 |
| cg22945413 | chr1 | 65399413 | 65399414 | 32773 | JAK1 |
| cg23004466 | chr7 | 106815478 | 106815479 | 6019 | HBP1 |
| cg23017594 | chr14 | 32728466 | 32728467 | −55992 | RNU6-2 |
| cg23044884 | chr8 | 30245145 | 30245146 | −2229 | RBPMS-AS1 |
| cg23102014 | chr15 | 70574295 | 70574296 | −184040 | TLE3 |
| cg23157190 | chr2 | 75060880 | 75060881 | 1099 | HK2 |
| cg23311108 | chr5 | 115387951 | 115387952 | 789 | ARL14EPL |
| cg23486701 | chr2 | 54789491 | 54789492 | 3961 | SPTBN1 |
| cg23493018 | chr8 | 37309823 | 37309824 | 41607 | LOC100507420 |
| cg23544996 | chr3 | 182514833 | 182514834 | 3543 | ATP11B |
| cg23679141 | chr4 | 165118930 | 165118931 | −68 | ANP32C |
| cg23824801 | chr12 | 54653403 | 54653404 | −34 | CBX5 |
| cg23829949 | chr1 | 244214679 | 244214680 | 119 | ZBTB18 |
| cg23837109 | chr10 | 75670435 | 75670436 | −426 | PLAU |
| cg23857976 | chr17 | 56065481 | 56065482 | 133 | VEZF1 |
| cg23880736 | chr4 | 582172 | 582173 | −37190 | PDE6B |
| cg23981150 | chr1 | 161111090 | 161111091 | −8613 | DEDD |
| cg24056269 | chr13 | 99171636 | 99171637 | 2742 | STK24 |
| cg24065504 | chr10 | 90613015 | 90613016 | −1284 | ANKRD22 |
| cg24232444 | chr13 | 99545448 | 99545449 | 61111 | DOCK9-AS1 |
| cg24284882 | chr4 | 154418379 | 154418380 | 30882 | KIAA0922 |
| cg24304617 | chr1 | 169079632 | 169079633 | 3686 | ATP1B1 |
| cg24376286 | chr2 | 198245629 | 198245630 | 54141 | SF3B1 |
| cg24383056 | chr17 | 48071706 | 48071707 | 881 | DLX3 |
| cg24449629 | chr19 | 52646265 | 52646266 | −3075 | ZNF616 |
| cg24576298 | chr7 | 108137995 | 108137996 | 28766 | PNPLA8 |
| cg24640156 | chr2 | 132202427 | 132202428 | 39 | LOC401010 |
| cg24742520 | chr1 | 19506481 | 19506482 | 30264 | UBR4 |
| cg24783785 | chr17 | 619036 | 619037 | −941 | VPS53 |
| cg24958366 | chr17 | 46952555 | 46952556 | −17592 | ATP5G1 |
| cg25104397 | chr10 | 104535920 | 104535921 | 33 | WBP1L |
| cg25135755 | chr15 | 23894248 | 23894249 | −1256 | MAGEL2 |
| cg25195795 | chr10 | 21807252 | 21807253 | 7358 | SKIDA1 |
| cg25279586 | chr18 | 7566258 | 7566259 | −1055 | PTPRM |
| cg25364972 | chr2 | 217075573 | 217075574 | −6038 | PKI55 |
| cg25522867 | chr11 | 34236648 | 34236649 | 109538 | NAT10 |
| cg25683268 | chr17 | 53809564 | 53809565 | −83 | TMEM100 |
| cg25708982 | chr13 | 112895431 | 112895432 | 31882 | LOC101928730 |
| cg26009832 | chr1 | 169081894 | 169081895 | 5948 | ATP1B1 |
| cg26250154 | chr2 | 241562424 | 241562425 | −2237 | GPR35 |
| cg26401541 | chr6 | 91078974 | 91078975 | 56514 | MIR4464 |
| cg26405097 | chr6 | 15428301 | 15428302 | 179216 | JARID2 |
| cg26407558 | chr1 | 207262706 | 207262707 | 79 | C4BPB |
| cg26576206 | chr19 | 1064938 | 1064939 | −983 | HMHA1 |
| cg26923863 | chr4 | 1221838 | 1221839 | 17931 | CTBP1-AS |
| cg27010159 | chr12 | 119591747 | 119591748 | −24847 | HSPB8 |
| cg27057509 | chr6 | 30883762 | 30883763 | 1655 | VARS2 |
| cg27089675 | chr10 | 123838499 | 123838500 | −34054 | TACC2 |
| cg27090007 | chr13 | 28519388 | 28519389 | 46 | ATP5EP2 |
| cg27120934 | chr6 | 129480619 | 129480620 | 276334 | LAMA2 |
| cg27143049 | chr11 | 14665558 | 14665559 | 290 | PDE3B |
| cg27186013 | chr4 | 95264127 | 95264128 | −101 | HPGDS |
| cg27284331 | chr7 | 106297689 | 106297690 | 3944 | CCDC71L |
| cg27296330 | chr19 | 54041251 | 54041252 | −81 | ZNF331 |
| cg27408285 | chr12 | 54653364 | 54653365 | 5 | CBX5 |
| cg27457290 | chr2 | 64246845 | 64246846 | −632 | VPS54 |
| cg27544294 | chr22 | 25082493 | 25082494 | −27380 | POM121L10P |
| cg27588356 | chr6 | 161459571 | 161459572 | 46813 | MAP3K4 |
| cg27638509 | chr12 | 132093988 | 132093989 | −101643 | SFSWAP |
| cg27644327 | chr6 | 90845852 | 90845853 | 160774 | BACH2 |
| cg12649038 | chr10 | 116282534 | 116282535 | 4150 | ABLIM1 |
| cg15867698 | chr14 | 69438267 | 69438268 | 7815 | ACTN1 |
| cg01116137 | chr20 | 25034263 | 25034264 | 4554 | ACSS1 |
| cg02086310 | chr20 | 25039719 | 25039720 | −902 | ACSS1 |
| cg03461110 | chr7 | 4778881 | 4778882 | −36380 | AP5Z1 |
| cg10036013 | chr7 | 4778839 | 4778840 | −36422 | AP5Z1 |
| cg08826152 | chr17 | 15869607 | 15869608 | 21377 | ADORA2B |
| cg01921773 | chr16 | 75661691 | 75661692 | −4471 | ADAT1 |
| cg12789173 | chr11 | 118084192 | 118084193 | −119 | AMICA1 |
| cg18051353 | chr8 | 68251877 | 68251878 | 4034 | ARFGEF1 |
| cg22301128 | chr4 | 77011716 | 77011717 | 15869 | ART3 |
| cg15109018 | chr12 | 85862615 | 85862616 | 188580 | ALX1 |
| cg02536838 | chr8 | 108510343 | 108510344 | −90 | ANGPT1 |
| cg18031596 | chr8 | 108510292 | 108510293 | −39 | ANGPT1 |
| cg07065759 | chr2 | 198017462 | 198017463 | 149780 | ANKRD44-IT1 |
| cg24065504 | chr10 | 90613015 | 90613016 | −1284 | ANKRD22 |
| cg22143698 | chr5 | 10608058 | 10608059 | 43624 | ANKRD33B |
| cg09555124 | chr6 | 160451213 | 160451214 | −22518 | AIRN |
| cg11262262 | chr17 | 35305366 | 35305367 | −808 | AATF |
| cg22256433 | chr17 | 7942743 | 7942744 | 386 | ALOX15B |
| cg07145988 | chr1 | 8692312 | 8692313 | 185386 | RERE |
| cg17597631 | chr1 | 8443425 | 8443426 | 40321 | RERE |
| cg13286116 | chr11 | 13302098 | 13302099 | 2825 | ARNTL |
| cg21226442 | chr12 | 27088580 | 27088581 | 2673 | ASUN |
| cg19930116 | chr14 | 50809588 | 50809589 | 30542 | ATP5S |
| cg03976645 | chr7 | 16724981 | 16724982 | 39223 | BZW2 |
| cg03541331 | chr1 | 85786958 | 85786959 | −44372 | BCL10 |
| cg17173975 | chr12 | 32292997 | 32292998 | 32813 | BICD1 |
| cg10091662 | chr10 | 22609897 | 22609898 | −241 | BMI1 |
| cg14242995 | chr9 | 122249943 | 122249944 | −118205 | BRINP1 |
| cg21386573 | chr1 | 94219800 | 94219801 | −72407 | BCAR3 |
| cg23944804 | chr20 | 11871384 | 11871385 | 14 | BTBD3 |
| cg03035849 | chr6 | 91003200 | 91003201 | 3426 | BACH2 |
| cg26803268 | chr10 | 18549536 | 18549537 | −47 | CACNB2 |
| cg02849693 | chr19 | 54402455 | 54402456 | −13535 | CACNG7 |
| cg16894855 | chr10 | 12430878 | 12430879 | 39296 | CAMKID |
| cg00452133 | chr1 | 7308117 | 7308118 | 462734 | CAMTA1 |
| cg21833076 | chr11 | 104643591 | 104643592 | 125805 | CASP12 |
| cg10185424 | chr5 | 66478491 | 66478492 | 14125 | CD180 |
| cg12880685 | chr10 | 120489658 | 120489659 | 25099 | CACUL1 |
| cg25188006 | chr3 | 350503 | 350504 | −10862 | CHL1 |
| cg14276584 | chr9 | 99318213 | 99318214 | 10989 | CDC14B |
| cg15145341 | chr13 | 25506340 | 25506341 | −9314 | CENPJ |
| cg05759347 | chr1 | 243416723 | 243416724 | 1984 | CEP170 |
| cg27408285 | chr12 | 54653364 | 54653365 | 5 | CBX5 |
| cg03441844 | chr1 | 161368947 | 161368948 | −31275 | C1orf192 |
| cg06279274 | chr10 | 124635805 | 124635806 | −3343 | LOC399815 |
| cg02914652 | chr12 | 4417142 | 4417143 | −13216 | C12orf5 |
| cg25174412 | chr12 | 105803653 | 105803654 | 79240 | C12orf75 |
| cg12777448 | chr14 | 58618986 | 58618987 | −140 | C14orf37 |
| cg19095568 | chr15 | 41062113 | 41062114 | −45 | C15orf62 |
| cg26594335 | chr5 | 76010472 | 76010473 | −1395 | F2R |
| cg19795616 | chr7 | 106371890 | 106371891 | −70257 | CCDC71L |
| cg27576694 | chr7 | 106372161 | 106372162 | −70528 | CCDC71L |
| cg01992590 | chr17 | 48277042 | 48277043 | 1957 | COL1A1 |
| cg03544320 | chr4 | 5894691 | 5894692 | 118 | CRMP1 |
| cg26407558 | chr1 | 207262706 | 207262707 | 79 | C4BPB |
| cg02717454 | chr16 | 3928799 | 3928800 | 1321 | CREBBP |
| cg13308137 | chr11 | 47528955 | 47528956 | −12883 | CELF1 |
| cg15009198 | chr2 | 97429502 | 97429503 | 2864 | CNNM4 |
| cg14140403 | chr4 | 908952 | 908953 | 17221 | GAK |
| cg16218221 | chr2 | 208576609 | 208576610 | 346 | CCNYL1 |
| cg01366985 | chr6 | 25167695 | 25167696 | −29076 | CMAHP |
| cg08306955 | chr6 | 25137971 | 25137972 | 79 | CMAHP |
| cg26325335 | chr3 | 50402333 | 50402334 | −431 | CYB561D2 |
| cg04771084 | chr6 | 31973255 | 31973256 | −103 | CYP21A2 |
| cg12727605 | chr6 | 33292029 | 33292030 | −1237 | DAXX |
| cg03911306 | chr3 | 16648294 | 16648295 | −1289 | DAZL |
| cg06488150 | chr7 | 6476003 | 6476004 | 11639 | DAGLB |
| cg08313420 | chr7 | 6476110 | 6476111 | 11532 | DAGLB |
| cg05512157 | chr12 | 50901878 | 50901879 | 3111 | DIP2B |
| cg04083575 | chr7 | 153748818 | 153748819 | −684 | DPP6 |
| cg02490460 | chr8 | 1365502 | 1365503 | −84029 | DLGAP2 |
| cg24383056 | chr17 | 48071706 | 48071707 | 881 | DLX3 |
| cg27207470 | chr11 | 111848326 | 111848327 | 294 | DIXDC1 |
| cg15302376 | chr2 | 25560263 | 25560264 | 4520 | DNMT3A |
| cg24232444 | chr13 | 99545448 | 99545449 | 61111 | DOCK9-AS1 |
| cg06098530 | chr10 | 76727919 | 76727920 | 90352 | DUPD1 |
| cg15514380 | chr21 | 38737243 | 38737244 | −2615 | DYRK1A |
| cg19988492 | chr21 | 38807712 | 38807713 | 15111 | DYRK1A |
| cg13896699 | chr5 | 13770231 | 13770232 | 174357 | DNAH5 |
| cg18370682 | chr5 | 158239759 | 158239760 | 287028 | EBF1 |
| cg13679714 | chr17 | 77706946 | 77706947 | 2082 | ENPP7 |
| cg11909467 | chr8 | 132912348 | 132912349 | −4007 | EFR3A |
| cg17741339 | chr6 | 152085619 | 152085620 | −41188 | ESR1 |
| cg18132851 | chr6 | 152085641 | 152085642 | −41166 | ESR1 |
| cg05304366 | chr15 | 40226905 | 40226906 | 581 | EIF2AK4 |
| cg02015053 | chr15 | 44853982 | 44853983 | 24717 | EIF3J |
| cg02556954 | chr5 | 137848577 | 137848578 | 28822 | ETF1 |
| cg22747380 | chr8 | 118993090 | 118993091 | 130967 | EXT1 |
| cg04536922 | chr4 | 89978566 | 89978567 | −221 | FAM13A |
| cg25779483 | chr4 | 89978300 | 89978301 | 45 | FAM13A |
| cg15704219 | chr10 | 5735135 | 5735136 | 8335 | FAM208B |
| cg24729928 | chr12 | 31480184 | 31480185 | −1026 | FAM60A |
| cg18182216 | chr1 | 150978385 | 150978386 | 887 | FAM63A |
| cg19140262 | chr6 | 99380488 | 99380489 | 15393 | FBXL4 |
| cg13912027 | chr11 | 72759293 | 72759294 | 93849 | FCHSD2 |
| cg22303909 | chr7 | 50518439 | 50518440 | −352 | FIGNL1 |
| cg03546163 | chr6 | 35654363 | 35654364 | 2328 | FKBP5 |
| cg01927745 | chr5 | 72677723 | 72677724 | 66628 | FOXD1 |
| cg08038033 | chr3 | 71354056 | 71354057 | −146 | FOXP1 |
| cg22589728 | chr3 | 71439885 | 71439886 | −85975 | FOXP1 |
| cg11955727 | chr2 | 84105546 | 84105547 | −412259 | FUNDC2P2 |
| cg17765025 | chr2 | 84105169 | 84105170 | −412636 | FUNDC2P2 |
| cg24070198 | chr6 | 37014597 | 37014598 | 41175 | FGD2 |
| cg26250154 | chr2 | 241562424 | 241562425 | −2237 | GPR35 |
| cg04864807 | chr2 | 121412139 | 121412140 | −142727 | GLI2 |
| cg00167275 | chr10 | 88854588 | 88854589 | 187 | GLUD1 |
| cg24616553 | chr3 | 113557638 | 113557639 | −42 | GRAMD1C |
| cg17988310 | chr22 | 40355732 | 40355733 | 12912 | GRAP2 |
| cg16292016 | chr5 | 42424356 | 42424357 | −197 | GHR |
| cg08644463 | chr1 | 110106962 | 110106963 | 15777 | GNAI3 |
| cg06445016 | chr8 | 61835848 | 61835849 | 44458 | LOC100130298 |
| cg01254303 | chr12 | 119592035 | 119592036 | −24559 | HSPB8 |
| cg27010159 | chr12 | 119591747 | 119591748 | −24847 | HSPB8 |
| cg27186013 | chr4 | 95264127 | 95264128 | −101 | HPGDS |
| cg20995304 | chr12 | 48196167 | 48196168 | 17595 | HDAC7 |
| cg01405107 | chr17 | 46671635 | 46671636 | −533 | HOXB5 |
| cg18273840 | chr5 | 45695643 | 45695644 | 576 | HCN1 |
| cg01305421 | chr12 | 102874286 | 102874287 | 91 | IGF1 |
| cg06652329 | chr12 | 102874566 | 102874567 | −189 | IGF1 |
| cg27300829 | chr13 | 48811111 | 48811112 | 3838 | ITM2B |
| cg01044293 | chr2 | 173296469 | 173296470 | 4156 | ITGA6 |
| cg09122035 | chr11 | 319667 | 319668 | 1246 | IFITM3 |
| cg26015683 | chr6 | 29720519 | 29720520 | −1595 | IFITM4P |
| cg09324669 | chr1 | 234749105 | 234749106 | −3835 | IRF2BP2 |
| cg14898127 | chr15 | 81587493 | 81587494 | −1760 | IL16 |
| cg10530883 | chr5 | 3596207 | 3596208 | 40 | IRX1 |
| cg22945413 | chr1 | 65399413 | 65399414 | 32773 | JAK1 |
| cg15046123 | chr6 | 15421581 | 15421582 | 172496 | JARID2 |
| cg26405097 | chr6 | 15428301 | 15428302 | 179216 | JARID2 |
| cg14734614 | chr19 | 51473346 | 51473347 | −418 | KLK6 |
| cg02193146 | chr1 | 110752257 | 110752258 | 351 | KCNC4-AS1 |
| cg14326196 | chr9 | 116860650 | 116860651 | 686 | KIF12 |
| cg05157625 | chr14 | 93153553 | 93153554 | 61493 | LGMN |
| cg16259904 | chr10 | 134146220 | 134146221 | 480 | LRRC27 |
| cg23771949 | chr10 | 134165390 | 134165391 | 14780 | LRRC27 |
| cg17718703 | chr1 | 90313059 | 90313060 | 25580 | LRRC8D |
| cg11556164 | chr7 | 110738315 | 110738316 | 7254 | LRRN3 |
| cg24453118 | chr13 | 47229927 | 47229928 | 102632 | LRCH1 |
| cg06168204 | chr6 | 27570548 | 27570549 | −91265 | LINC01012 |
| cg00431894 | chr4 | 189871012 | 189871013 | 494281 | LINC01060 |
| cg17837517 | chr4 | 189541174 | 189541175 | 164443 | LINC01060 |
| cg24680439 | chr10 | 134778467 | 134778468 | 325 | LINC01166 |
| cg00399683 | chr7 | 153109375 | 153109376 | −57 | LINC01287 |
| cg05132077 | chr22 | 49448320 | 49448321 | 185739 | LINC01310 |
| cg00500229 | chr1 | 243054071 | 243054072 | 210974 | LINC01347 |
| cg12691488 | chr1 | 243053673 | 243053674 | 211372 | LINC01347 |
| cg15000827 | chr9 | 110228655 | 110228656 | 210 | LINC01509 |
| cg02991085 | chr20 | 30073537 | 30073538 | −43 | LINC00028 |
| cg25502144 | chr20 | 30073546 | 30073547 | −34 | LINC00028 |
| cg24169486 | chr13 | 106971568 | 106971569 | −57342 | LINC00460 |
| cg07019386 | chr20 | 47013687 | 47013688 | 25034 | LINC00494 |
| cg16763089 | chr20 | 5485284 | 5485285 | −43 | LINC00654 |
| cg07385778 | chr3 | 72320634 | 72320635 | 120227 | LINC00870 |
| cg02939781 | chr3 | 183208857 | 183208858 | 43419 | LINC00888 |
| cg20197130 | chr12 | 127256717 | 127256718 | 90 | LINC00944 |
| cg18128914 | chr15 | 74244249 | 74244250 | −23661 | LOXL1-AS1 |
| cg06477663 | chr13 | 46757415 | 46757416 | −957 | LCP1 |
| cg09750084 | chr13 | 49005868 | 49005869 | −4826 | LPAR6 |
| cg25310233 | chr1 | 31234437 | 31234438 | −3755 | LAPTM5 |
| cg03764364 | chr10 | 29480551 | 29480552 | −97438 | LYZL1 |
| cg06822816 | chr8 | 120220882 | 120220883 | 273 | MAL2 |
| cg04116354 | chr1 | 26003643 | 26003644 | 59685 | MAN1C1 |
| cg10555744 | chr1 | 25946258 | 25946259 | 2300 | MAN1C1 |
| cg27406664 | chr17 | 2294951 | 2294952 | 9306 | MNT |
| cg00014638 | chr10 | 94113651 | 94113652 | 62732 | 5-Mar |
| cg07834396 | chr10 | 23385979 | 23385980 | 1553 | MSRB2 |
| cg25100962 | chr12 | 31782808 | 31782809 | −17285 | METTL20 |
| cg02132714 | chr17 | 46656690 | 46656691 | 618 | MIR10A |
| cg17144149 | chr17 | 46656572 | 46656573 | 736 | MIR10A |
| cg02322400 | chr11 | 95980186 | 95980187 | −94415 | MIR1260B |
| cg14858267 | chr3 | 44037760 | 44037761 | −117943 | MIR138-1 |
| cg03853208 | chr7 | 25989763 | 25989764 | −158 | MIR148A |
| cg23299919 | chr7 | 157406096 | 157406097 | −38983 | MIR153-2 |
| cg26963367 | chr15 | 89157841 | 89157842 | −2687 | MIR3529 |
| cg00772991 | chr2 | 220716794 | 220716795 | 54491 | MIR4268 |
| cg21222426 | chr9 | 20339790 | 20339791 | 71445 | MIR4473 |
| cg25891647 | chr11 | 123232359 | 123232360 | 19860 | MIR4493 |
| cg00815832 | chr1 | 228658973 | 228658974 | 9199 | MIR4666A |
| cg06850005 | chr10 | 35926564 | 35926565 | 3615 | MIR4683 |
| cg12583076 | chr12 | 65082713 | 65082714 | −66329 | MIR548Z |
| cg24919348 | chr8 | 100549849 | 100549850 | −761 | MIR875 |
| cg08113187 | chr16 | 87469329 | 87469330 | 43529 | MAP1LC3B |
| cg10341310 | chr8 | 66582206 | 66582207 | 99 | MTFR1 |
| cg25461186 | chr12 | 122518089 | 122518090 | 1456 | MLXIP |
| cg07052063 | chr10 | 99255236 | 99255237 | 3129 | MMS19 |
| cg21092324 | chr4 | 90816310 | 90816311 | 259 | MMRN1 |
| cg12299554 | chr15 | 94840953 | 94840954 | −476 | MCTP2 |
| cg07249730 | chr17 | 55362836 | 55362837 | 28463 | MSI2 |
| cg27098685 | chr3 | 151867537 | 151867538 | −118291 | MBNL1 |
| cg12970155 | chr19 | 54374873 | 54374874 | 2229 | MYADM |
| cg03909800 | chr6 | 76458005 | 76458006 | −887 | MYO6 |
| cg24405716 | chr15 | 31280513 | 31280514 | 3293 | MTMR10 |
| cg11231949 | chr8 | 63161616 | 63161617 | 116 | NKAIN3 |
| cg12161228 | chr11 | 89224506 | 89224507 | 226 | NOX4 |
| cg27113419 | chr16 | 58533979 | 58533980 | −67 | NDRG4 |
| cg20585841 | chr8 | 102729926 | 102729927 | 73512 | NCALD |
| cg10549831 | chr10 | 5488366 | 5488367 | −147 | NET1 |
| cg20478129 | chr14 | 27067372 | 27067373 | −413 | NOVA1 |
| cg05348875 | chr2 | 206628625 | 206628626 | 81402 | NRP2 |
| cg07381872 | chr1 | 61408076 | 61408077 | 28371 | NFIA-AS2 |
| cg20781967 | chr12 | 772688 | 772689 | 218 | NINJ2 |
| cg22675447 | chr1 | 24745395 | 24745396 | 3151 | NIPAL3 |
| cg13404054 | chr19 | 15311666 | 15311667 | 125 | NOTCH3 |
| cg00434461 | chr5 | 92905860 | 92905861 | 1188 | NR2F1-AS1 |
| cg04998202 | chr1 | 61545546 | 61545547 | −1987 | NFIA |
| cg22656550 | chr2 | 132202485 | 132202486 | −19 | LOC401010 |
| cg11718162 | chr1 | 154128002 | 154128003 | −411 | NUP210L |
| cg00404641 | chr3 | 131080516 | 131080517 | −172 | NUDT16P1 |
| cg05107535 | chr16 | 3242850 | 3242851 | −11396 | OR1F1 |
| cg10909506 | chr17 | 38081995 | 38081996 | 1888 | ORMDL3 |
| cg17775490 | chr20 | 45179354 | 45179355 | −142 | OCSTAMP |
| cg05554346 | chr4 | 4145468 | 4145469 | 83152 | OTOP1 |
| cg14762436 | chr7 | 24917750 | 24917751 | 14489 | OSBPL3 |
| cg11157127 | chr6 | 143998869 | 143998870 | −232 | PHACTR2 |
| cg11080540 | chr5 | 54897272 | 54897273 | −66367 | PPAP2A |
| cg14914552 | chr8 | 97340188 | 97340189 | 66075 | PTDSS1 |
| cg06895913 | chr5 | 58957910 | 58957911 | −75587 | PDE4D |
| cg18804667 | chr5 | 58883392 | 58883393 | −1069 | PDE4D |
| cg23880736 | chr4 | 582172 | 582173 | −37190 | PDE6B |
| cg26402555 | chr14 | 105750534 | 105750535 | −16613 | PACS2 |
| cg05460226 | chr17 | 8804279 | 8804280 | 11554 | PIK3R5 |
| cg18214661 | chr8 | 17471997 | 17471998 | 38056 | PDGFRL |
| cg02976588 | chr1 | 150135546 | 150135547 | 13377 | PLEKHO1 |
| cg27247736 | chr6 | 160241105 | 160241106 | 19825 | PNEDC1 |
| cg27544294 | chr22 | 25082493 | 25082494 | −27380 | POM121L10P |
| cg20587236 | chr12 | 109900956 | 109900957 | 14198 | KCTD10 |
| cg26475911 | chr17 | 73056187 | 73056188 | 12909 | KCTD2 |
| cg03942932 | chr6 | 106441441 | 106441442 | −92753 | PRDM1 |
| cg19266387 | chr3 | 183596123 | 183596124 | 6569 | PARL |
| cg25459280 | chr12 | 124492549 | 124492550 | 34788 | ZNF664-FAM101A |
| cg25353287 | chr2 | 42277667 | 42277668 | 2507 | PKDCC |
| cg24631428 | chr6 | 64281604 | 64281605 | −312 | PTP4A1 |
| cg00898013 | chr13 | 113819073 | 113819074 | 6106 | PROZ |
| cg13435137 | chr17 | 3814718 | 3814719 | 5241 | P2RX1 |
| cg21816330 | chr17 | 27044629 | 27044630 | 278 | RAB34 |
| cg12019814 | chr8 | 117861247 | 117861248 | −25415 | RAD21-AS1 |
| cg11958644 | chr5 | 130872422 | 130872423 | 98506 | RAPGEF6 |
| cg10167378 | chr1 | 228756711 | 228756712 | −23682 | RHOU |
| cg15514896 | chr1 | 229074115 | 229074116 | 203292 | RHOU |
| cg16512390 | chr1 | 228756714 | 228756715 | −23679 | RHOU |
| cg26856443 | chr13 | 114890296 | 114890297 | 7798 | RASA3 |
| cg02152108 | chr22 | 37641506 | 37641507 | −1168 | RAC2 |
| cg14419424 | chr10 | 65388604 | 65388605 | 107482 | REEP3 |
| cg14376836 | chr9 | 94606638 | 94606639 | 105805 | ROR2 |
| cg14362178 | chr2 | 79007750 | 79007751 | −245061 | REG3G |
| cg10196532 | chr13 | 50134640 | 50134641 | 25078 | RCBTB1 |
| cg13260278 | chr10 | 121265587 | 121265588 | 30457 | RGS10 |
| cg17766305 | chr10 | 90147030 | 90147031 | 196051 | RNLS |
| cg01124132 | chr22 | 32599511 | 32599512 | −48 | RFPL2 |
| cg12427303 | chr22 | 32599613 | 32599614 | −150 | RFPL2 |
| cg12906381 | chr22 | 32599516 | 32599517 | −53 | RFPL2 |
| cg13405775 | chr22 | 32599648 | 32599649 | −185 | RFPL2 |
| cg22404498 | chr22 | 32600722 | 32600723 | −5 | RFPL2 |
| cg15011899 | chr13 | 111854118 | 111854119 | 14946 | ARHGEF7 |
| cg05084827 | chr2 | 55402999 | 55403000 | −56039 | RPS27A |
| cg25673720 | chr17 | 74188601 | 74188602 | 47788 | RNF157 |
| cg09696535 | chr22 | 32810284 | 32810285 | −2011 | RTCB |
| cg05217983 | chr6 | 45406867 | 45406868 | 16554 | RUNX2 |
| cg18808261 | chr3 | 18464935 | 18464936 | 1893 | SATB1 |
| cg06816239 | chr1 | 169679199 | 169679200 | −1203 | SELL |
| cg01557792 | chr14 | 70162755 | 70162756 | −71073 | SRSF5 |
| cg24056269 | chr13 | 99171636 | 99171637 | 2742 | STK24 |
| cg20625523 | chr2 | 64893849 | 64893850 | −12804 | SERTAD2 |
| cg03400131 | chr6 | 134497247 | 134497248 | −178 | SGK1 |
| cg12315391 | chr3 | 157815145 | 157815146 | 8806 | SHOX2 |
| cg08946713 | chr2 | 191844998 | 191844999 | 33977 | STAT1 |
| cg04398282 | chr4 | 68424256 | 68424257 | −189 | STAP1 |
| cg08641990 | chr1 | 54822503 | 54822504 | 49564 | SSBP3 |
| cg16924102 | chr4 | 20044588 | 20044589 | −210598 | SLIT2 |
| cg07912766 | chr18 | 45458698 | 45458699 | −1182 | SMAD2 |
| cg19193595 | chr15 | 67396487 | 67396488 | −21566 | SMAD3 |
| cg13466988 | chr1 | 12538541 | 12538542 | −28758 | SNORA59A |
| cg26876834 | chr16 | 2013573 | 2013574 | −467 | SNORA64 |
| cg06568880 | chr17 | 2166583 | 2166584 | 2909 | SMG6 |
| cg11065621 | chr8 | 82606061 | 82606062 | 1145 | SLC10A5 |
| cg12099423 | chr20 | 61590751 | 61590752 | 6753 | SLC17A9 |
| cg17221813 | chr20 | 61590823 | 61590824 | 6825 | SLC17A9 |
| cg07850527 | chr16 | 89268040 | 89268041 | −1512 | SLC22A31 |
| cg23824902 | chr1 | 9619882 | 9619883 | 20355 | SLC25A33 |
| cg25964728 | chr3 | 136539328 | 136539329 | 1468 | SLC35G2 |
| cg12549858 | chr5 | 101425870 | 101425871 | 206382 | SLCO4C1 |
| cg26465602 | chr16 | 1098847 | 1098848 | −23908 | SSTR5 |
| cg19841369 | chr14 | 64663928 | 64663929 | −16930 | SYNE2 |
| cg21487856 | chr2 | 54828502 | 54828503 | 42972 | SPTBN1 |
| cg23486701 | chr2 | 54789491 | 54789492 | 3961 | SPTBN1 |
| cg03204322 | chr1 | 84767878 | 84767879 | −455 | SAMD13 |
| cg21384492 | chr2 | 241938321 | 241938322 | 67 | SNED1 |
| cg10184328 | chr7 | 138349158 | 138349159 | −190 | SVOPL |
| cg16111924 | chr7 | 138348981 | 138348982 | −13 | SVOPL |
| cg02863594 | chr6 | 33280199 | 33280200 | 1964 | TAPBP |
| cg02142483 | chr16 | 84560555 | 84560556 | −22268 | TLDC1 |
| cg09259081 | chr16 | 84538889 | 84538890 | −602 | TLDC1 |
| cg16496269 | chr16 | 84541118 | 84541119 | −2831 | TLDC1 |
| cg10890302 | chr6 | 32064246 | 32064247 | 12904 | TNXB |
| cg10923662 | chr6 | 32064258 | 32064259 | 12892 | TNXB |
| cg13401703 | chr15 | 99789777 | 99789778 | 46 | TTC23 |
| cg08280368 | chr14 | 71110536 | 71110537 | 2033 | TTC9 |
| cg02710015 | chr12 | 55362424 | 55362425 | 5091 | TESPA1 |
| cg24536818 | chr12 | 55371892 | 55371893 | 3729 | TESPA1 |
| cg00052964 | chr2 | 85135823 | 85135824 | 3061 | TMSB10 |
| cg10061361 | chr4 | 122078167 | 122078168 | 7327 | TNIP3 |
| cg00804338 | chr13 | 114239234 | 114239235 | 179 | TFDP1 |
| cg03215181 | chr4 | 122873487 | 122873488 | −579 | TRPC3 |
| cg14774438 | chr11 | 111957396 | 111957397 | 125 | TIMM8B |
| cg15756407 | chr11 | 111956086 | 111956087 | 1435 | TIMM8B |
| cg11692124 | chr17 | 79316097 | 79316098 | −11624 | TMEM105 |
| cg15331834 | chr10 | 45360969 | 45360970 | −45794 | TMEM72 |
| cg07721852 | chr11 | 118576628 | 118576629 | −26248 | TREH |
| cg04656070 | chr8 | 116661063 | 116661064 | 19175 | TRPS1 |
| cg18297196 | chr6 | 41168941 | 41168942 | −17 | TREML2 |
| cg20606062 | chr7 | 99517279 | 99517280 | −57 | TRIM4 |
| cg18417954 | chr19 | 55672513 | 55672514 | −3414 | TNNI3 |
| cg08566455 | chr2 | 130971164 | 130971165 | −15131 | TUBA3E |
| cg10274682 | chr19 | 6496041 | 6496042 | 6553 | TUBB4A |
| cg08123444 | chr2 | 9833101 | 9833102 | −61918 | YWHAQ |
| cg24742520 | chr1 | 19506481 | 19506482 | 30264 | UBR4 |
| cg20222519 | chr3 | 23245916 | 23245917 | 1133 | UBE2E2 |
| cg22374742 | chr2 | 106761673 | 106761674 | −6337 | UXS1 |
| cg02314201 | chr10 | 134843775 | 134843776 | 56425 | LOC100128127 |
| cg24142603 | chr8 | 72753888 | 72753889 | −1469 | LOC100132891 |
| cg21358380 | chr2 | 70353785 | 70353786 | −1338 | LOC100133985 |
| cg24716416 | chr4 | 188736112 | 188736113 | −142318 | LOC100506272 |
| cg03894796 | chr8 | 144361315 | 144361316 | 2554 | LOC100507316 |
| cg17372657 | chr7 | 1216933 | 1216934 | 16513 | LOC101927021 |
| cg15720112 | chr5 | 125036315 | 125036316 | 207362 | LOC101927460 |
| cg10584024 | chr1 | 84234998 | 84234999 | 91230 | LOC101927560 |
| cg10918327 | chr8 | 9106953 | 9106954 | 60445 | LOC101929128 |
| cg17335387 | chr5 | 55828740 | 55828741 | −51145 | LOC102467147 |
| cg05429448 | chr3 | 101659630 | 101659631 | −72 | LOC152225 |
| cg07772781 | chr3 | 101798142 | 101798143 | 138440 | LOC152225 |
| cg12963656 | chr3 | 101659687 | 101659688 | −15 | LOC152225 |
| cg01413790 | chr19 | 35330180 | 35330181 | −6408 | LOC400685 |
| cg15695738 | chr19 | 35329860 | 35329861 | −6088 | LOC400685 |
| cg17786894 | chr2 | 65131556 | 65131557 | 28024 | LOC400958 |
| cg03568507 | chr2 | 240153791 | 240153792 | −36639 | MGC16025 |
| cg09681977 | chr2 | 240153103 | 240153104 | −35951 | MGC16025 |
| cg18766900 | chr10 | 11574616 | 11574617 | −338 | USP6NL |
| cg01832672 | chr12 | 123358583 | 123358584 | 22128 | VPS37B |
| cg08479516 | chr7 | 158905536 | 158905537 | 32112 | VIPR2 |
| cg22668906 | chr11 | 128180077 | 128180078 | 212127 | ETS1 |
| cg01359822 | chr21 | 40176597 | 40176598 | −633 | ETS2 |
| cg00168785 | chr2 | 160142643 | 160142644 | 419 | WDSUB1 |
| cg20769177 | chr17 | 44928516 | 44928517 | −451 | WNT9B |
| cg25104397 | chr10 | 104535920 | 104535921 | 33 | WBP1L |
| cg16306870 | chr3 | 194868790 | 194868791 | 191 | XXYLT1-AS2 |
| cg19760965 | chr3 | 194868843 | 194868844 | 244 | XXYLT1-AS2 |
| cg02750262 | chr18 | 72916776 | 72916777 | 4504 | ZADH2 |
| cg22088248 | chr18 | 72917387 | 72917388 | 3893 | ZADH2 |
| cg23829949 | chr1 | 244214679 | 244214680 | 119 | ZBTB18 |
| cg25784220 | chr19 | 58609602 | 58609603 | 127 | ZSCAN18 |
| cg12856392 | chr7 | 64126140 | 64126141 | −320 | ZNF107 |
| cg13939291 | chr4 | 383159 | 383160 | 51564 | ZNF141 |
| cg12868738 | chr7 | 148946070 | 148946071 | 9329 | ZNF212 |
| cg21918548 | chr8 | 145956024 | 145956025 | 24945 | ZNF251 |
| cg15598244 | chr1 | 23696413 | 23696414 | −57 | ZNF436 |
| cg00674365 | chr19 | 57019069 | 57019070 | −142 | ZNF471 |
| cg13904970 | chr5 | 123987667 | 123987668 | 93137 | ZNF608 |
| cg04192168 | chr15 | 64806741 | 64806742 | 15123 | ZNF609 |
| cg03151810 | chr8 | 144371745 | 144371746 | −1813 | ZNF696 |
| cg03692651 | chr19 | 22444593 | 22444594 | −24658 | ZNF729 |
| cg26827373 | chr19 | 12175935 | 12175936 | 390 | ZNF844 |
| cg00257775 | chr6 | 37904681 | 37904682 | 117375 | ZFAND3 |
| cg15747825 | chr6 | 28565626 | 28565627 | −10515 | ZBED9 |
| cg11706775 | chr1 | 52608467 | 52608468 | 702 | ZFYVE9 |
| cg07266910 | chr3 | 178745575 | 178745576 | 44080 | ZMAT3 |
| cg19768229 | chr5 | 60615309 | 60615310 | −12790 | ZSWIM6 |
| cg09639931 | chr17 | 38024394 | 38024395 | −60 | ZPBP2 |
| cg16810031 | chr17 | 38024146 | 38024147 | −308 | ZPBP2 |

As used herein, the term “penalized regression” refers to a statistical method aimed at identifying the smallest number of predictors required to predict an outcome out of a larger list of biomarkers as implemented for example in the R statistical package “penalized” as described in Goeman, J. J., L1 penalized estimation in the Cox proportional hazards model. Biometrical Journal 52(1), 70-84.

As used herein, the term “clustering” refers to the grouping of a set of objects in such a way that objects in the same group (called a cluster) are more similar (in some sense or another) to each other than to those in other groups (clusters).

As used herein, the term “Hierarchical clustering” refers to a statistical method that builds a hierarchy of “clusters” based on how similar (close) or dissimilar (distant) are the clusters from each other as described for example in Kaufman, L.; Rousseeuw, P. J. (1990). Finding Groups in Data: An Introduction to Cluster Analysis (1 ed.). New York: John Wiley. ISBN 0-471-87876-6.

As used herein, the term “gene pathways” refers to a group of genes that encode proteins that are known to interact with each other in physiological pathways or processes. These pathways are characterized using bio-computational methods such as Ingenuity Pathway Analysis.

As used herein, the term “Receiver operating characteristics (ROC) assay” refers to a statistical method that creates a graphical plot that illustrates the performance of a predictor. The true positive rate of prediction is plotted against the false positive rate at various threshold settings for the predictor (i.e., different % of methylation) as described for example in Hanley, James A.; McNeil, Barbara J. (1982). “The Meaning and Use of the Area under a Receiver Operating Characteristic (ROC) Curve”. Radiology 143 (1): 29-36.

As used herein, the term “Multivariate linear regression” refers to a statistical method that estimates the relationship between multiple “independent variables” or “predictors” such as percentage of methylation, age, sex etc. and an “outcome” or a “dependent variable” such as cancer or stage of cancer. This method determines the statistical significance of each “predictor” (independent variable) in predicting the “outcome” (dependent variable) when several “independent variables” are included in the model.

Methods or means for detecting the “DNA methylation level” are well known in the art, for example, pyrosequencing as described in {Zhang Y, Petropoulos S, Liu J, Cheishvili D, Zhou R, Dymov S, Li K, Li N, Szyf M. The signature of liver cancer in immune cells DNA methylation. Clin Epigenetics. 2018 Jan. 18; 10:8}; targeted amplification of bisulfite converted DNA and next generation sequencing as described in {El-Zein M, Cheishvili D, Gotlieb W, Gilbert L, Hemmings R, Behr M A, Szyf M, Franco E L; MARKER study group. Genome-wide DNA methylation profiling identifies two novel genes in cervical neoplasia. Int J Cancer. 2020 Sep 1;147(5):1264-1274}; methylated DNA immunoprecipitation followed by quantitative PCR as described in {Provençal N, Suderman M J, Guillemin C, Massart R, Ruggiero A, Wang D, Bennett A J, Pierre P J, Friedman D P, Côté S M, Hallett M, Tremblay R E, Suomi S J, Szyf M. The signature of maternal rearing in the methylome in rhesus macaque prefrontal cortex and T cells. J Neurosci. 2012 Oct. 31; 32(44):15626-42}; methylation specific PCR as described in {Ku J L, Jeon Y K, Park J G. Methylation-specific PCR. Methods Mol Biol. 2011; 791:23-32}; high resolution melting PCR (HRM) as described in {Stefanska B, Bouzelmat A, Huang J, Suderman M, Hallett M, Han Z G, Al-Mahtab M, Akbar S M, Khan W A, Raqib R, Szyf M. Discovery and validation of DNA hypomethylation biomarkers for liver cancer using HRM-specific probes. PLoS One. 2013 Aug. 7; 8(8): e68439}; sequenome mass array technology as described in {Song F, Mahmood S, Ghosh S, Liang P, Smiraglia D J, Nagase H, Held W A. Tissue specific differentially methylated regions (TDMR): Changes in DNA methylation during development. Genomics. 2009 February; 93(2):130-91.

### BRIEF DESCRIPTIONS OF THE DRAWINGS

FIGS. 1A-1B. Genome wide distribution of cancer specific DNA methylation signatures in peripheral blood mononuclear cells. FIG. 1A. A genome wide view (IGV genome browser) of the escalating differences in DNA methylation from healthy controls (Ref.), chronic hepatitis B (HepB) and C (HepC), and progressive stages of HCC (CAN1, CAN2, CANS, CAN4). FIG. 1B. The top box plot represents beta values of DNA methylation of sites that lose methylation as HCC progresses. The bottom box plot represents beta values of DNA methylation of sites that gain DNA methylation during progression of HCC.

FIG. 2. DNA methylation signature of HCC progression in 69 individuals which are in the state of normal, chronic hepatitis and stages of HCC. Each column represents a subject, each row represents a CG site, level of methylation is indicated by gray level. Black represents most methylated, white represents least methylated and grey represents intermediate methylated.

FIG. 3A. Overlap in number of CG sites that are differentially methylated between stages of HCC (CAN1, CAN2, CAN3, CAN4). FIG. 3B. Number of CGs that become either hypo or hypermethylated during HCC progression (CAN1, CAN2, CAN3, CAN4).

FIG. 4. Prediction of 49 chronic hepatitis and HCC patients using the DNA methylation signature derived for stage 1 HCC (20 patients). Black represents most methylated, white represents least methylated and grey represents intermediate methylated.

FIG. 5. Prediction of 49 chronic hepatitis and HCC patients using the DNA methylation signature derived for stage 2 HCC (20 patients). Black represents most methylated, white represents least methylated and grey represents intermediate methylated.

FIG. 6. Prediction of 49 chronic hepatitis and HCC patients using the DNA methylation signature derived for stage 3 HCC (20 patients). Black represents most methylated, white represents least methylated and grey represents intermediate methylated.

FIG. 7. Prediction of 49 chronic hepatitis and HCC patients using the DNA methylation signature derived for stage 4 HCC (20 patients). Black represents most methylated, white represents least methylated and grey represents intermediate methylated.

FIG. 8. Prediction of 69 controls, chronic hepatitis and HCC patients using the 350 CG DNA methylation signature (Table 3). Black represents most methylated, white represents least methylated and grey represents intermediate methylated.

FIG. 9. Prediction of 69 controls, chronic hepatitis and HCC patients using a 31 CG DNA methylation signature (Table 5). Black represents most methylated, white represents least methylated and grey represents intermediate methylated.

FIG. 10A. Prediction (0 to 1 probability) differentiating stage HCC 2-4 from stage 1 using measurements of DNA methylation of following predictive CGs described in the present disclsoure, Target CG IDs: cg03252499, cg03481488, cg04398282, cg10203922, cg11783497, cg13710613, cg14762436, cg23486701. FIG. 10B. Prediction (0 to 1 probability) differentiating stage HCC 3-4 from stage 1 and 2 using measurements of DNA methylation of following predictive CGs described in the present disclosure, Target CG IDs: cg02914652, cg03252499, cg11783497, cg11911769, cg12019814, cg14711743, cg15607708, cg20956548, cg22876402, cg24958366. FIG. 10C. Prediction (0 to 1 probability) differentiating stage HCC 4 from stage 1 to 3 using measurements of DNA methylation in predictive CGs described in the present disclsoure, Target CG IDs: cg02782634, cg11151251, cg24958366, cg06874640, cg27284331, cg16476382, cg14711743.

FIG. 11. Differences in DNA methylation profiles between T cells from healthy controls (n=10; TCTRL-1 to TCTRL-10) and HCC stages (n=10; TCAN1, TCAN2, TCAN3, TCAN4).

FIG. 12. Prediction of HCC using measurements of DNA methylation in PBMC DNA of the 370 CGs derived from T cells (Table 6).

FIG. 13A. Prediction of HCC using measurements of DNA methylation in T cell DNA of 350 CGs derived from PBMC DNA (Table 3). FIG. 13B. Overlap between differentially methylated CGs in T cell DNA from different stages of HCC (TCAN1-4) and in DNA from PBMC from different stages of HCC (PBMCCAN1, PBMCCAN2, PBMCCAN4). FIG. 13C. Prediction of HCC using measurements of DNA methylation in T cell DNA of 31 CGs derived from PBMC DNA (Table 5).

FIGS. 14A-14D. Validation by pyrosequencing of differences in DNA methylation in 4 genes between all control samples and early stages of HCC in T cell DNA from a replication set.

FIG. 15A. HCC versus healthy controls (T cells Illumina) and FIG. 15B. HCC versus all controls (PB MC Illumina). Receiver Operating Characteristic (ROC) measuring specificity (fraction of true positives) (Y axis) and sensitivity (absence of false positives) (X axis) of STAP1 methylation as a biomarker for discriminating HCC from healthy controls using T cells DNA (Illumina 450K data) (FIG. 15A) or HCC from all controls (healthy and chronic hepatitis) in PBMC (FIG. 15B).

FIG. 16A. HCC versus healthy controls (pyro) and FIG. 16B. HCC versus all controls (pyro). Receiver Operating Characteristic (ROC) measuring specificity (Y axis) and sensitivity (X axis) of STAP1 methylation (measured using pyrosequencing) in T cells as a biomarker for discriminating

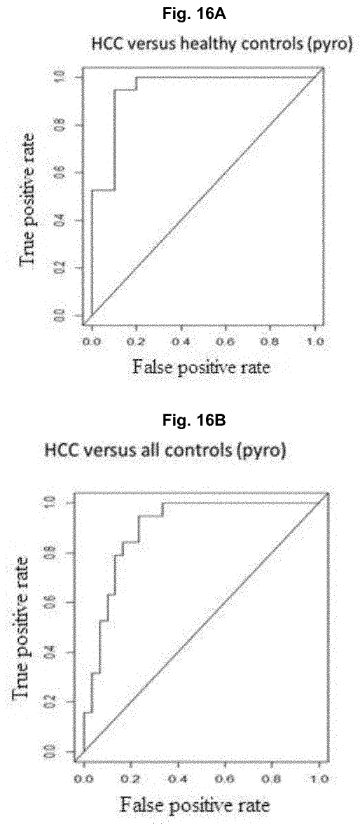

HCC from healthy controls (FIG. 16A) and all controls (FIG. 16B).

### DETAILED DESCRIPTIONS

### Embodiment 1. DNA Methylation Signatures in Peripheral Blood Mononuclear Cells (PBMC) that Correlate with HCC Cancer Stages

### Patient Samples

HCC staging was diagnosed according to EASL-EORTC Clinical Practice Guidelines:

Management of hepatocellular carcinoma. The patients were divided into four groups, including Stage 0 (1), stage A (2), stage B (3) and stage C+D (4). For simplicity, stages 1-4 are referenced in the figures and embodiments. Chronic hepatitis B diagnosing was confirmed using AASLD practice guideline for chronic Hepatitis B, and chronic hepatitis C diagnosing was according to AASLD recommendations for testing, managing and treating Hepatitis C. A strict exclusion criterion was any other known inflammatory disease (bacterial or viral infection with the exception of hepatitis B or C, diabetes, asthma, autoimmune disease, active thyroid disease) which could alter T cells and monocytes characteristics. Clinical characteristics of patients are provided in Table 1 and 2. The participants in the study provided consent according to the regulations of the Capital Medical School. The study received ethical approval from The Capital Medical School in Beijing and McGill University (IRB Study Number A02-M34-13B).

TABLE 1
Clinical data of training cohort.

| | | | | | | anticancer | AFP | HBV- | HCV- |
|---|---|---|---|---|---|---|---|---|---|
| ID | sex | age | diagnosis | smoking | alcohol | therapy | (ng/ml) | DNA | RNA |
| 1_9 | M | 52 | HCC-BCLC-0 | No | 15 y | TACE | 5.8 | <500 | |
| 1_6 | M | 45 | HCC-BCLC-0 | No | No | No | 2.25 | <500 | |
| 1_5 | M | 55 | HCC-BCLC-0 | 20 y | No | No | 25.83 | 4.80E+04 | |
| 1_10 | M | 61 | HCC-BCLC-0 | No | 30 y | TACE | 81.98 | <500 | |
| 1_8 | M | 44 | HCC-BCLC-0 | 25 y | No | No | 50.12 | 1.26E+04 | |
| 1_2 | M | 59 | HCC-BCLC-0 | 15 y | seldom | No | 7.34 | <500 | |
| 1_1 | M | 52 | HCC-BCLC-0 | No | No | No | 4.72 | 2.46E+05 | <1000 |
| 1_7 | M | 58 | HCC-BCLC-0 | No | No | Iodine/ | 1.75 | 5.41E+02 | |
| | | | | | | Metuximab | | | |
| 1_3 | M | 47 | HCC-BCLC-0 | 20 y | 20 y | No | 3.07 | 3.92E+05 | |
| 1_4 | F | 56 | HCC-BCLC-0 | No | seldom | No | 13.4 | <500 | 586000 |
| 2_8 | F | 50 | HCC-BCLC-A | No | No | TACE + ADV- | 9307 | <500 IU/ml | |
| | | | | | | TK, Sorafenib | | | |
| 2_3 | M | 55 | HCC-BCLC-A | quit | No | TACE + RFA | 5.01 | <500 | |
| 2_4 | M | 56 | HCC-BCLC-A | quit | 30 y | TACE | 325.2 | 5.41E+04 | |
| 2_1 | M | 46 | HCC-BCLC-A | quit | seldom | No | 0.82 | <500 | |
| 2_2 | M | 34 | HCC-BCLC-A | No | seldom | No | 3176 | 1.08E+04 | |
| 2_10 | M | 70 | HCC-BCLC-A | No | No | TACE + RFA | 50.79 | | <1000 |
| 2_5 | M | 73 | HCC-BCLC-A | No | No | No | 16.38 | <500 | |
| 2_6 | M | 41 | HCC-BCLC-A | seldom | seldom | hepatectomy + | 2.31 | 8.59E+02 | |
| | | | | | | RFA | | | |
| 2_7 | F | 53 | HCC-BCLC-A | No | seldom | RFA | 117.4 | 1.08E+06 | |
| 2_9 | M | 44 | HCC-BCLC-A | 25 y | No | Iodine/ | 32.76 | <500 | |
| | | | | | | Metuximab | | | |
| 3_8 | M | 52 | HCC-BCLC-B | No | No | TACE + RFA | 46761 | <500 | |
| 3_10 | M | 59 | HCC-BCLC-B | No | No | TACE + RFA | 86.72 | 2.70E+05 | |
| 3_3 | M | 60 | HCC-BCLC-B | 40 y | No | TACE | 43583 | 4.61E+08 | |
| 3_9 | M | 53 | HCC-BCLC-B | 30 y | 30 y | No | 3481 | 7.47E+05 | |
| 3_1 | M | 53 | HCC-BCLC-B | 30 y | 20 y | TACE | 254.3 | 1.18E+03 | |
| 3_7 | M | 46 | HCC-BCLC-B | 25 y | 25 y | No | 6.2 | <500 | |
| 3_4 | M | 66 | HCC-BCLC-B | quit | 40 y | TACE | 28.84 | 4.26E+04 | |
| 3_5 | M | 55 | HCC-BCLC-B | quit | 30 y | TACE | 4616 | <500 | |
| 3_6 | F | 59 | HCC-BCLC-B | No | No | hepatectomy | 1.25 | <500 | |
| 3_2 | M | 58 | HCC-BCLC-B | No | 30 y | TACE | 31474 | 5.25E+04 | |
| 4_3 | M | 48 | HCC-BCLC- | No | 5 y | hepatectomy | 1087 | <500 | |
| | | | C + D | | | | | | |
| 4_7 | M | 48 | HCC-BCLC- | No | No | TACE + RFA | 1304 | <500 | |
| | | | C + D | | | | | | |
| 4_2 | M | 58 | HCC-BCLC- | quit | 30 y | No | 67.44 | | |
| | | | C + D | | | | | | |
| 4_5 | F | 47 | HCC-BCLC- | No | No | hepatectomy + | 4325 | <500 | |
| | | | C + D | | | PECT + RFA | | | |
| 4_8 | M | 37 | HCC-BCLC- | 20 y | seldom | No | 97.91 | 1.30E+05 | |
| | | | C + D | | | | | | |
| 4_9 | M | 76 | HCC-BCLC- | 50 y | seldom | TACE + RFA | 1.89 | <500 | |
| | | | C + D | | | | | | |
| 4_1 | F | 28 | HCC-BCLC- | No | No | hepatectomy | 44740 | <500 | |
| | | | C + D | | | | | | |
| 4_4 | M | 59 | HCC-BCLC- | No | No | RFA | 12.51 | 6.92E+02 | |
| | | | C + D | | | | | | |
| 4_6 | M | 31 | HCC-BCLC- | No | No | hepatectomy + | 2.3 | 4.16E+03 | |
| | | | C + D | | | RFA | | | |
| C1 | M | 47 | hepatitis C | No | No | | 2.65 | | |
| C6 | M | 54 | hepatitis C | No | No | | 1.66 | | |
| C4 | M | 31 | hepatitis C | 10 y | No | | 2.68 | | |
| C7 | F | 43 | hepatitis C | No | seldom | | 2.78 | | |
| C5 | M | 57 | hepatitis C | No | No | | 4.35 | | |
| C2 | M | 33 | hepatitis C | 10 y | No | | 4.43 | | |
| C10 | F | 26 | hepatitis C | No | No | | | | |
| C8 | F | 41 | hepatitis C | 10 y | No | | 1.5 | | |
| C9 | M | 28 | hepatitis C | No | seldom | | 2.09 | | |
| C3 | M | 17 | hepatitis C | No | No | | 1.56 | | |
| B3 | M | 53 | hepatitis B | 30 y | No | | 25 | 1.77E+05 | |
| B4 | M | 19 | hepatitis B | No | No | | | 1.85E+07 | |
| B2 | M | 36 | hepatitis B | No | 10 y | | 3686 | 4.85E+05 | |
| B7 | M | 43 | hepatitis B | 30 y | No | | 48.42 | 2.02E+08 | |
| B5 | M | 42 | hepatitis B | 20 y | seldom | | 99.1 | 6.01E+04 | |
| B1 | M | 40 | hepatitis B | 10 y | 25 y | | 199.8 | 2.09E+04 | |
| B8 | F | 31 | hepatitis B | No | No | | 17.72 | 2.55E+04 | |
| B9 | M | 37 | hepatitis B | No | No | | 48.34 | 1.29E+04 | |
| B6 | M | 38 | hepatitis B | 10 y | 14 y | | 2.78 | 1.09E+03 | |
| B10 | F | 30 | hepatitis B | No | No | | | 7.83E+02 | |
| H1 | M | 30 | healthy | 10 y | seldom | | | | |
| H2 | F | 28 | healthy | No | No | | | | |
| H3 | M | 40 | healthy | 18 y | seldom | | | | |
| H4 | F | 42 | healthy | No | No | | | | |
| H5 | F | 53 | healthy | No | No | | | | |
| H6 | F | 25 | healthy | No | No | | | | |
| H7 | F | 33 | healthy | No | No | | | | |
| H8 | F | 28 | healthy | No | No | | | | |
| H9 | F | 36 | healthy | No | No | | | | |
| H10 | M | 29 | healthy | No | No | | | | |

DNA was prepared from PBMC cells for all patients. T cells were isolated from all healthy controls and from HCC patients (patient IDs; 1-1, 1-3, 1-6, 2-2, 2-3, 2-4, 3-6, 4-2, 4-3).

TABLE 2
Clinical data of test (replication) cohort

| | | | | | | anticancer | AFP | HBV- | HCV- |
|---|---|---|---|---|---|---|---|---|---|
| ID | sex | age | diagnosis | smoking | alcohol | therapy | (ng/ml) | DNA | RNA |
| I-11 | M | 68 | HCC-BCLC-0 | No | No | No | | <500 | |
| I-14 | M | 50 | HCC-BCLC-0 | 35 y | No | No | 1.53 | <500 | |
| I-18 | M | 65 | HCC-BCLC-0 | 50 y | No | No | 1.69 | <500 | |
| I-19 | M | 80 | HCC-BCLC-0 | No | No | No | 15.67 | <500 | |
| I-22 | M | 57 | HCC-BCLC-0 | 30 y | 30 y | No | 3.13 | <500 | |
| I-23 | M | 62 | HCC-BCLC-0 | No | No | No | 2355 | 10100000 | |
| I-24 | M | 54 | HCC-BCLC-0 | 20 y | No | No | | | |
| I-30 | M | 58 | HCC-BCLC-0 | No | No | No | 2.86 | <500 | |
| I-17 | M | 57 | HCC-BCLC-A | No | No | No | 1210 | <500 | |
| I-27 | M | 54 | HCC-BCLC-A | No | 40 y | No | 5.07 | 2720000 | |
| I-28 | M | 72 | HCC-BCLC-A | 30 y | No | No | 128.3 | <500 | |
| I-13 | M | 41 | HCC-BCLC-A | No | No | No | 1.51 | <500 | |
| I-12 | M | 43 | HCC-BCLC-A | No | No | No | 91.67 | <500 | |
| I-15 | M | 71 | HCC-BCLC-A | quit | No | No | 59.11 | <500 | |
| I-16 | F | 54 | HCC-BCLC-A | No | No | No | 4578 | <500 | |
| I-20 | M | 58 | HCC-BCLC-A | No | No | No | 3.01 | <500 | |
| I-25 | F | 68 | HCC-BCLC-A | No | No | No | 0.8 | <500 | |
| I I-11 | M | 47 | HCC-BCLC-A | 20 y | 10 y | No | 974.3 | <500 | |
| I I-13 | M | 45 | HCC-BCLC-A | 20 y | 17 y | No | 5.9 | <500 | |
| I I-15 | M | 62 | HCC-BCLC-A | No | seldom | No | 41.87 | <500 | |
| I-21 | M | 45 | HCC-BCLC-B | 20 y | 20 y | No | 852.3 | 3600 | |
| I I-12 | M | 53 | HCC-BCLC-B | 20 y | 20 y | No | 9.67 | 4190000 | |
| I I-14 | M | 64 | HCC-BCLC-B | 40 y | 40 y | No | 442.3 | 383000 | |
| I I-16 | M | 52 | HCC-BCLC-B | 30 y | 20 y | No | 37.05 | <500 | |
| I I-17 | M | 47 | HCC-BCLC-B | 30 y | 20 y | No | 2.54 | | |
| I I-18 | M | 52 | HCC-BCLC-B | 40 y | 30 y | No | 4.35 | 1620 | |
| I I-19 | M | 49 | HCC-BCLC-B | 30 y | No | No | 4565 | 3020 | |
| I I-20 | M | 45 | HCC-BCLC-B | No | No | No | 171.4 | 17600 | |
| III-16 | M | 54 | HCC-BCLC-B | 40 y | No | No | 358.5 | 1400000 | |
| III-17 | M | 34 | HCC-BCLC-B | 10 y | 5 y | No | 41524 | 8200 | |
| III-18 | M | 45 | HCC-BCLC-B | No | 20 y | No | 796.6 | <500 | |
| I-26 | M | 63 | HCC-BCLC-C | No | No | No | 7399 | <500 | |
| I-29 | M | 47 | HCC-BCLC-C | No | No | No | 12.46 | 470000 | |
| III-13 | M | 50 | HCC-BCLC-C | 10 y | 10 y | No | 56.88 | 1070 | |
| III-14 | M | 51 | HCC-BCLC-C | 30 y | 20 y | No | 37182 | <500 | |
| III-15 | F | 53 | HCC-BCLC-C | No | No | No | 3.64 | 172000 | |
| III-19 | M | 60 | HCC-BCLC-C | 40 y | seldom | No | 30512 | <500 | |
| IV-13 | M | 56 | HCC-BCLC-C | quit | 10 y | No | 230.9 | <500 | |
| IV-15 | M | 29 | HCC-BCLC-C | 10 y | No | No | 121000 | 2410 | |
| IV-16 | M | 63 | HCC-BCLC-C | 20 y | 20 y | No | 4282 | 394000 | |
| IV-17 | M | 64 | HCC-BCLC-C | No | No | No | 243.6 | 2700 | |
| IV-18 | M | 42 | HCC-BCLC-C | No | No | No | 4.95 | 1640 | |
| IV-19 | M | 50 | HCC-BCLC-C | 20 y | 17 y | No | 1382 | 2350000 | |
| IV-20 | M | 50 | HCC-BCLC-C | No | 30 years | No | 4040 | <500 | |
| III-11 | M | 57 | HCC-BCLC-D | 40 y | 40 y | No | 496.4 | <500 | |
| III-12 | M | 55 | HCC-BCLC-D | 30 y | 30 y | No | 23.47 | 1080 | |
| III-20 | F | 72 | HCC-BCLC-D | quit | No | No | 4.8 | 965000 | |
| IV-11 | M | 53 | HCC-BCLC-D | quit | 30 y | No | 6.88 | 1800 | |
| IV-12 | F | 62 | HCC-BCLC-D | No | No | No | 10.56 | 8080000 | |
| IV-14 | M | 42 | HCC-BCLC-D | 20 y | No | No | 745.9 | 215000 | |
| B11 | M | 54 | hepatitis B | No | No | | 181.9 | 2.64E+07 | |
| B12 | F | 24 | hepatitis B | No | No | | 0.94 | <500 | |
| B13 | M | 26 | hepatitis B | 5 y | No | | 11.47 | 3.07E+04 | |
| B14 | M | 39 | hepatitis B | No | No | | 3 | <500 | |
| B15 | M | 55 | hepatitis B | 30 y | No | | 6.54 | <500 | |
| B16 | M | 63 | hepatitis B | No | No | | 20.73 | 2.19E+07 | |
| B17 | M | 61 | hepatitis B | 40 y | No | | 4.67 | <500 | |
| B18 | F | 27 | hepatitis B | No | No | | 35.2 | 1.22E+08 | |
| B19 | M | 34 | hepatitis B | No | No | | 160.7 | 4.78E+03 | |
| B20 | F | 56 | hepatitis B | quit | No | | 4.26 | <500 | |
| C11 | M | 19 | hepatitis C | No | No | | 1.72 | | 2.01E+06 |
| C12 | F | 51 | hepatitis C | No | No | | 8.67 | | 1.25E+06 |
| C13 | M | 32 | hepatitis C | No | No | | 3.12 | <500 | 9.56E+05 |
| C14 | M | 60 | hepatitis C | 30 y | No | | 37.98 | | 1.87E+06 |
| C15 | M | 57 | hepatitis C | 30 y | 20 y | | 4.25 | | |
| C16 | F | 52 | hepatitis C | No | No | | 4.25 | | 2..22E+05 |
| C17 | F | 48 | hepatitis C | No | No | | 1.82 | | 9.66E+06 |
| C18 | F | 62 | hepatitis C | No | No | | 2.44 | | 1.98E+07 |
| C19 | M | 69 | hepatitis C | No | quit | | 3.08 | | <100 |
| C20 | F | 51 | hepatitis C | No | No | | 3.4 | | 6.40E+04 |
| H11 | M | 31 | healthy | | | | | | |
| H12 | M | 37 | healthy | | | | | | |
| H13 | M | 25 | healthy | | | | | | |
| H14 | M | 44 | healthy | | | | | | |
| H15 | M | 38 | healthy | | | | | | |
| H16 | F | 42 | healthy | | | | | | |
| H17 | F | 44 | healthy | | | | | | |
| H18 | F | 23 | healthy | | | | | | |
| H19 | M | 39 | healthy | | | | | | |
| H20 | F | 32 | healthy | | | | | | |

AFP—alpha feto protein;
HBV—Hepatitis B virus;
HCV—hepatitis C virus;
TACE—transcatheter arterial chemoembolization;
RFA—Radiofrequency ablation

### Illumina Beadchip 450K Analysis

Blood was drawn from patients into EDTA coated tubes and peripheral blood mononuclear cells were isolated using standard protocols by centrifugation on Ficoll-Hypaque density gradient and mononuclear cells were collected on top of the Ficoll-Hypaque layer because they have a lower density using routine lab procedures, mononuclear cells were separated from platelets by washing (46). DNA was extracted from the cells using commercial human DNA extraction kits (Qiagen), DNA was bisulfite converted and subjected to Illumina HumanMethyaltion450k BeadChip hybridization and scanning using standard protocols recommended by the manufacturer. Samples were randomized with respect to slide and position on arrays and all samples were hybridized and scanned concurrently to mitigate batch effects as recommended by McGill Genome Quebec innovation center according to Illumina Infinum HD technology user guide. Illumina arrays hybridizations and scanning were performed by the McGill Genome Quebec Innovation center according to the manufacturer guidelines. Illumina arrays were analyzed using the ChAMP Bioconductor package in R(47). IDAT files were used as input in the champ.load function using minfi quality control and normalization options. Raw data were filtered for probes with a detection value of P>0.01in at least one sample. Probes on the X or Y chromosome are filtered out to mitigate sex effects and probes with SNPs as identified in (48), as well as probes that align to multiple locations as identified in (48). Batch effects were analyzed on the non-normalized data using the function champ.svd. Five out of the first 6 principal components were associated with group and batch (slides). Intra-array normalization to adjust the data for bias introduced by the Infinium type 2 probe design was performed using beta-mixture quantile normalization (BMIQ) with function champ.norm (norm =“BMIQ”) (47). Batch effects are corrected after BMIQ normalization using champ.runcombat function.

Cell count analysis for peripheral blood mononuclear cells distribution in samples was performed according to the Houseman algorithm (49) using the function estimate Cell Counts and FlowSorted.Blood.450k data as reference. The Beta values of the batch corrected normalized data are used for downstream statistical analyses.

To compute linear correlation between HCC stages and quantitative distribution of DNA methylation at the 450K CG sites, Pearson correlation between the normalized DNA methylation values and stages of HCC (with stage codes of 0 for control 1 and 2 for hepatitis B and C respectively and 3-6 for the 4 stages of HCC) is performed using the pearson corr function in R and correcting for multiple testing using the method “fdr” of Benjamini Hochberg (adjusted P value (Q) of <0.05) as well as the conservative Bonferroni correction (Q<1×10⁻⁷). A similar approach could be used utilizing new generations of Illumina arrays such as Illumina 850K arrays.

### Correlation Between Quantitative Distribution of Site-Specific DNA Methylation Levels and Progression of HCC

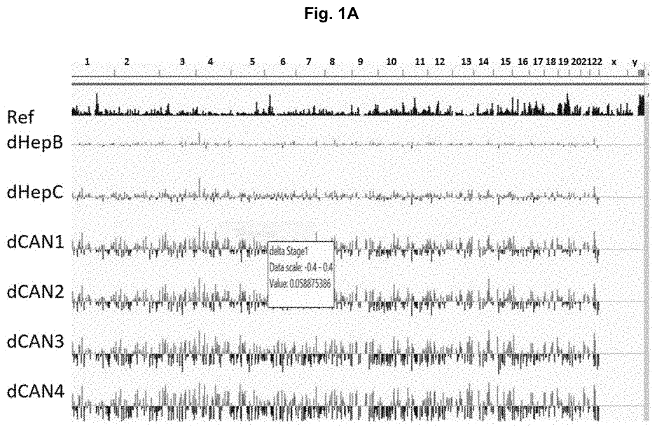

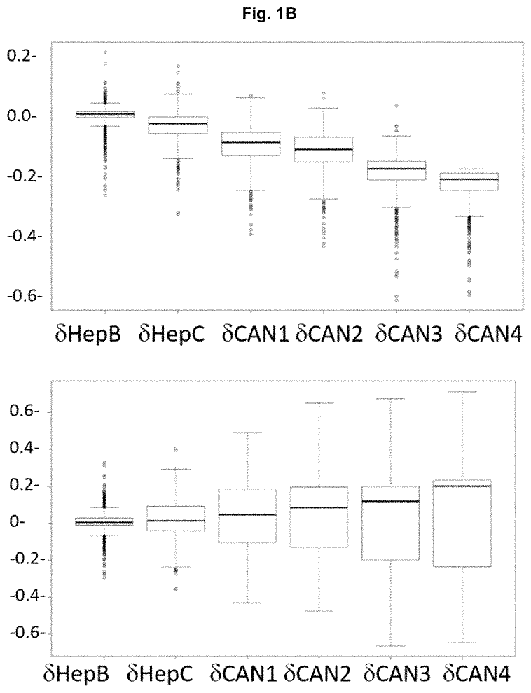

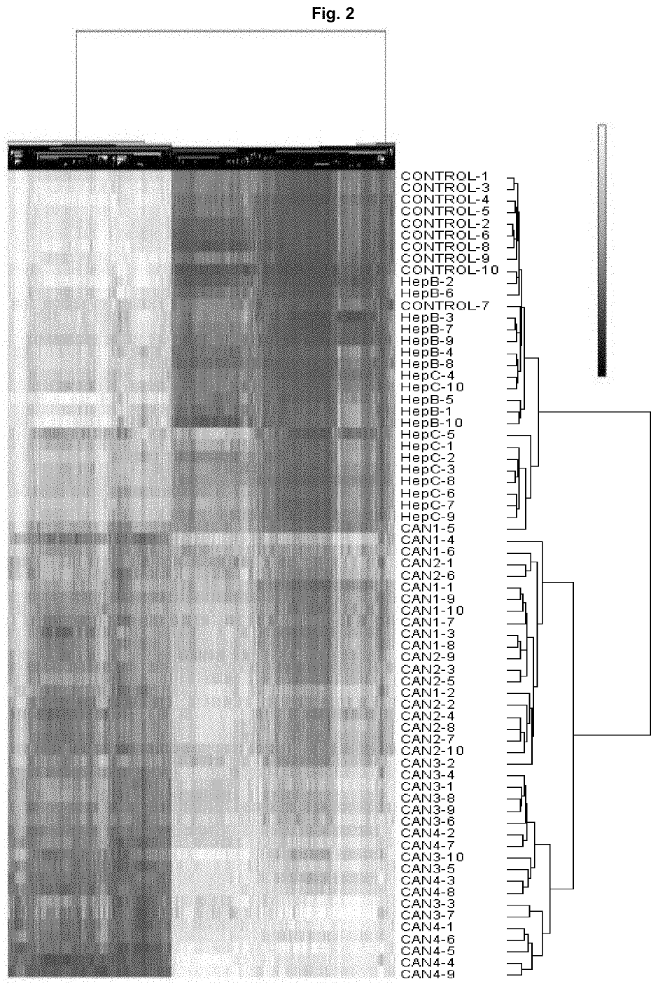

The analysis reveals a broad signature of DNA methylation that correlates with progression of HCC (160,904 sites). The analysis focuses on 3924 sites with the most robust changes (r>0.8; r<-0.8; delta beta>0.2/, delta beta>−0.2, p<10⁻⁷). A genome wide view of the intensifying changes in DNA methylation of these sites during HCC progression relative to chronic hepatitis B and C and control is shown in FIG. 1A. A box plot of the DNA methylation levels of sites that either increase or decrease methylation during HCC confirms the progression of changes in DNA methylation with progression of HCC with an increase in the extent of hypomethylation with progression of HCC (FIG. 1B). Clustering using One minus Pearson correlation reveals that these sites cluster all individual HCC patients away from control and Hepatitis B and C individuals with the exception of patient CAN1-5 who is clustered on the boundary between HepC and HCC, showing strong consistency across individual members of the different groups (FIG. 2).

Utility of DNA Methylation Signature of HCC in Peripheral Blood Mononuclear Cells for Differentiating Cancer Samples from Controls

These DNA methylation signatures have therefore the utility of classifying the stage of HCC in patient sample. The heat map in FIG. 2 reveals the intensification of the changes in DNA methylation differences with progression of HCC. Importantly, the combination of the analyses disclosure herein show that DNA methylation signatures differentiate individual HCC patients at the earliest stage from Hepatitis B and C which is a critical challenge in early diagnosis of HCC. Further, the analysis disclosed herein shows that changes in DNA methylation in PBMC from HCC patients could be distinguished from changes induced by viral triggered chronic inflammation. Based on the present disclosure any person skilled in the art may be able to derive similar DNA methylation signatures for other cancers.

### Embodiment 2. Unique and Overlapping Differentially Methylated Sites Associate with Different HCC Stages and Differentiate HCC from Hepatitis B and C

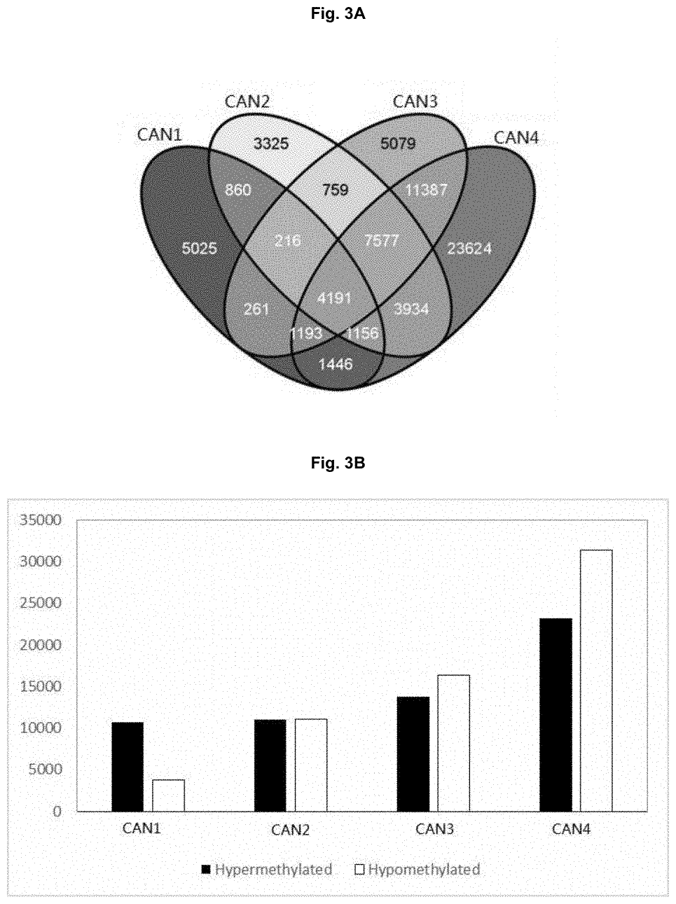

Differentially methylated CGs were delineated independently between healthy controls and each of the HCC stages using the Bioconductor package Limma (50) as implemented in ChAMP. The number of differentially methylated CG sites (p<1×10⁻⁷) between each stage of HCC and healthy controls increases with advance in stages; 14375 for stage 1, 22018 stage 2, 30709, stage 3 and 54580 for stage 4. Significance of overlap between two groups was determined using hypergeometric Fisher exact test in R. There is a significant overlap between the stages of cancer (FIG. 3A) suggesting common markers are affected in all HCC stages (p<1.9e⁻²⁹⁷).

The fraction of sites that are hypomethylated relative to hypermethylated sites in HCC increases as well from 26% in stage 1 to 57% in stage 4 (FIG. 3B). This increase in number of hypomethylated sites with progression of HCC was observed as well in the results of the Pearson correlation analysis (FIGS. 1A-1B & 2). For each HCC stage, a set of highly robust CG methylation markers are derived by using the threshold of p<1×10⁻⁷ (genome wide significance after Bonferroni correction) and delta beta of +/−0.3 for HCC stage 1 and p<10⁻¹⁰ delta beta of +/−0.3 for the stages 2-4 (a more stringent threshold for later stages is used to reduce the number of sites used for analysis) which were used for further analysis (74 for stage 1, 14 for stage 2, 58 for stage 3, and 298 for stage 4). By combining the lists of markers derived independently for each stage and removing redundant CG sites between stages, a combined non-redundant list of 350 CGs (Table 3) is derived.

TABLE 3
List of top significant 350 CG IDs derived from PBMC DNA that are differentially
methylated between stages of HCC and healthy controls.

| | | | | | |
|---|---|---|---|---|---|
| cg05375333 | cg24304617 | cg08649216 | cg15775914 | cg06098530 | cg04536922 |
| cg23679141 | cg26009832 | cg06908855 | cg21585138 | cg15514380 | cg20838429 |
| cg01546046 | cg27090007 | cg11412036 | cg00744866 | cg19988492 | cg21542922 |
| cg10036013 | cg24958366 | cg23824801 | cg08306955 | cg00361155 | cg11356004 |
| cg12829666 | cg17479131 | cg27408285 | cg15009198 | cg05423018 | cg19140262 |
| cg15011899 | cg27644327 | cg01810593 | cg18878210 | cg13710613 | cg05033369 |
| cg02001279 | cg11031737 | cg19795616 | cg02717454 | cg07072643 | cg09048334 |
| cg15188939 | cg09800500 | cg27284331 | cg22344162 | cg04018625 | cg04385818 |
| cg23311108 | cg02313495 | cg08575688 | cg26923863 | cg01238991 | cg01214050 |
| cg09789584 | cg16324306 | cg05486191 | cg15447825 | cg17741339 | cg14361741 |
| cg22301128 | cg02914652 | cg04171808 | cg04771084 | cg18132851 | cg16292016 |
| cg11737318 | cg11057824 | cg14276584 | cg23981150 | cg02556954 | cg14783904 |
| cg07118376 | cg26407558 | cg03496780 | cg24383056 | cg01359822 | cg26250154 |
| cg13978347 | cg09451574 | cg14375111 | cg24232444 | cg22747380 | cg02758552 |
| cg23544996 | cg21156970 | cg08944236 | cg22281935 | cg00211609 | cg21811450 |
| cg16306870 | cg01732538 | cg02142483 | cg22110158 | cg11911769 | cg03432151 |
| cg03731740 | cg10312296 | cg23102014 | cg04398282 | cg15755348 | cg08455089 |
| cg02749789 | cg17704839 | cg25683268 | cg08946713 | cg25195795 | cg17766305 |
| cg08123444 | cg24742520 | cg20460227 | cg24056269 | cg06151145 | cg06349546 |
| cg15747825 | cg14983135 | cg17163729 | cg15118835 | cg00568910 | cg23017594 |
| cg23829949 | cg21164050 | cg01417062 | cg14189441 | cg15146122 | cg12813441 |
| cg16712679 | cg06879746 | cg13146484 | cg16111924 | cg13615971 | cg01411912 |
| cg12820627 | cg27057509 | cg18417954 | cg27089675 | cg06194421 | cg15374754 |
| cg17534034 | cg23857976 | cg13913085 | cg07128102 | cg01966878 | cg00093544 |
| cg05591270 | cg05228338 | cg12705693 | cg18556587 | cg16565409 | cg14711743 |
| cg13219008 | cg24783785 | cg21579239 | cg02863594 | cg03044573 | cg00483304 |
| cg15607708 | cg27457290 | cg10274682 | cg08577341 | cg10469659 | cg24376286 |
| cg22475353 | cg14199837 | cg19389852 | cg12306086 | cg16240816 | cg27638509 |
| cg27296330 | cg25104397 | cg01839860 | cg21700582 | cg21487856 | cg11300809 |
| cg24449629 | cg20592700 | cg20222519 | cg14774438 | cg23486701 | cg09244071 |
| cg12177922 | cg27010159 | cg02272851 | cg15123819 | cg24640156 | cg00014638 |
| cg23004466 | cg14898127 | cg14734614 | cg00759807 | cg05086021 | cg00697672 |
| cg01696603 | cg11783497 | cg27120934 | cg07929642 | cg03899643 | cg01116137 |
| cg03639671 | cg08861115 | cg10078703 | cg08134863 | cg11556164 | cg20250700 |
| cg10203922 | cg15966610 | cg05099186 | cg20228731 | cg25135755 | cg15867698 |
| cg13749822 | cg13299325 | cg11767757 | cg23493018 | cg08113187 | cg11151251 |
| cg12263794 | cg22547775 | cg09545443 | cg04071270 | cg27588356 | cg05577016 |
| cg23157190 | cg22945413 | cg20427318 | cg20750319 | cg01611777 | cg01933228 |
| cg21406217 | cg15046123 | cg01698579 | cg12050434 | cg12299554 | cg11006453 |
| cg08247053 | cg26405097 | cg12691488 | cg00458932 | cg14356440 | cg03555836 |
| cg26576206 | cg03483626 | cg08568561 | cg25708982 | cg18482303 | cg02482718 |
| cg07212747 | cg14531436 | cg13943141 | cg12592365 | cg15323084 | cg24065504 |
| cg22872033 | cg20587236 | cg13619522 | cg19780570 | cg22876402 | cg09340198 |
| cg27186013 | cg24284882 | cg05502766 | cg20187173 | cg17092349 | cg22143698 |
| cg19851487 | cg17226602 | cg06445016 | cg07772781 | cg02782634 | cg07065759 |
| cg03481488 | cg22707529 | cg10895875 | cg01828328 | cg09987993 | cg21751540 |
| cg12598524 | cg19945957 | cg08634082 | cg05725404 | cg26401541 | cg20956548 |
| cg10761639 | cg05460226 | cg20944521 | cg14426660 | cg00248242 | cg18731803 |
| cg00350932 | cg25364972 | cg03252499 | cg04998202 | cg09514545 | cg09639931 |
| cg14914552 | cg00754989 | cg14762436 | cg07381872 | cg16476382 | cg16810031 |
| cg07504763 | cg01994308 | cg19266387 | cg14193653 | cg00189276 | cg10861953 |
| cg25279586 | cg23837109 | cg17934470 | cg22675447 | cg08858441 | cg12628061 |
| cg12019814 | cg10892950 | cg00758915 | cg09479286 | cg20874210 | cg06874640 |
| cg05941376 | cg02976588 | cg27143049 | cg00426720 | cg00321614 | cg15006843 |
| cg23044884 | cg24576298 | cg23880736 | cg05999692 | cg08226047 | cg25522867 |
| cg15891076 | cg12344600 | cg04090347 | cg10784548 | cg02265379 | cg01124132 |
| cg07145988 | cg27544294 | cg22515654 | cg12201380 | cg19925215 | cg10536529 |
| cg09635768 | cg00448395 | cg03062944 | cg05961707 | cg10995381 | cg16517298 |
| cg01124132 | cg10536529 | cg16517298 | cg18882449 | cg03909800 | cg18882449 |
| cg03909800 | | | | | |

HCC patients in the study and in clinical setting are a heterogeneous group with respect to alcohol, smoking (52-55), sex (56) and age (57) and each of these factors are known to affect DNA methylation. In addition, peripheral mononuclear cells are a heterogeneous mixture of cells and alterations in cell distribution between individuals might affect DNA methylation as well. In this study, the cell count distribution was first determined for each case using the Houseman algorithm (49). Two-way ANOVA followed by pairwise comparisons and correction for multiple testing found no significant difference in cell count between the groups. Multifactorial ANOVA with group, sex and age as cofactors was performed for CGs that were short listed for association with HCC using loop_anova lmFit function with Bonferoni adjustment for multiple testing. Multivariate linear regression was performed on the shortlisted CG sites that were found to associate with HCC to test whether these associations will survive if cell counts, sex, age, and alcohol abuse are used as covariates in the linear regression model using the lmFit function in R. Comparison of differentially methylated (relative to control) gene lists in different groups was performed using Venny. Hierarchical clustering was performed using One minus Pearson correlation and heatmaps were generated in the Broad institute GeneE application.

Then, a multivariate linear regression on the normalized beta values of the 350 CG sites is performed that differentiate HCC from all other groups using group (HCC versus non-HCC), sex, alcohol, smoking, age, and cell-count as covariates. All CG sites remained highly significant for the group covariate even after including the other covariates in the model. Following Bonferroni corrections for 350 measurements, 342 CG sites remained highly significant for group (HCC versus non-HCC). A multifactorial ANOVA analysis is performed on the beta values of the 350 sites as dependent variables and group (HCC versus non-HCC), sex and age as independent variables to determine whether there are possible interactions between either sex and group, age and group and between sex+age and group on DNA methylation.

While group remained significant for all 350 CGs no significant interactions with sex or age were found after Bonferroni corrections. In summary, these data show robust DNA methylation differences in PBMC DNA between HCC and other non-HCC patients including Hepatitis B and Hepatitis C.

### Embodiment 3. Utility of Cancer Stage Specific DNA Methylation Markers to Predict Unknown Samples from Patients Using One Minus Pearson Cluster Analysis, Detect Early Stages of HCC Cancer and Differentiate them from Chronic Hepatitis

The differentially methylated sites for each of the HCC stages were derived by comparing 10 healthy control and 10 stage specific HCCs. Other stages and the Hepatitis B and C samples were not “trained” (“trained” is used by the model to derive the differentially methylated sites) for these differentially methylated CGs and served as “cross-validation” sets of “unknown” samples to address the following questions: First, would the markers derived for one stage of cancer cluster correctly HCC samples that were not “trained” by these markers? Second, would DNA methylation markers that were “trained” to differentiate HCC from healthy controls also differentiate HCC from Hepatitis B and hepatitis C. Differentiating HCC from chronic hepatitis is a critical challenge for early diagnosis of HCC since a notable fraction of HCC patient progress from chronic hepatitis to HCC.

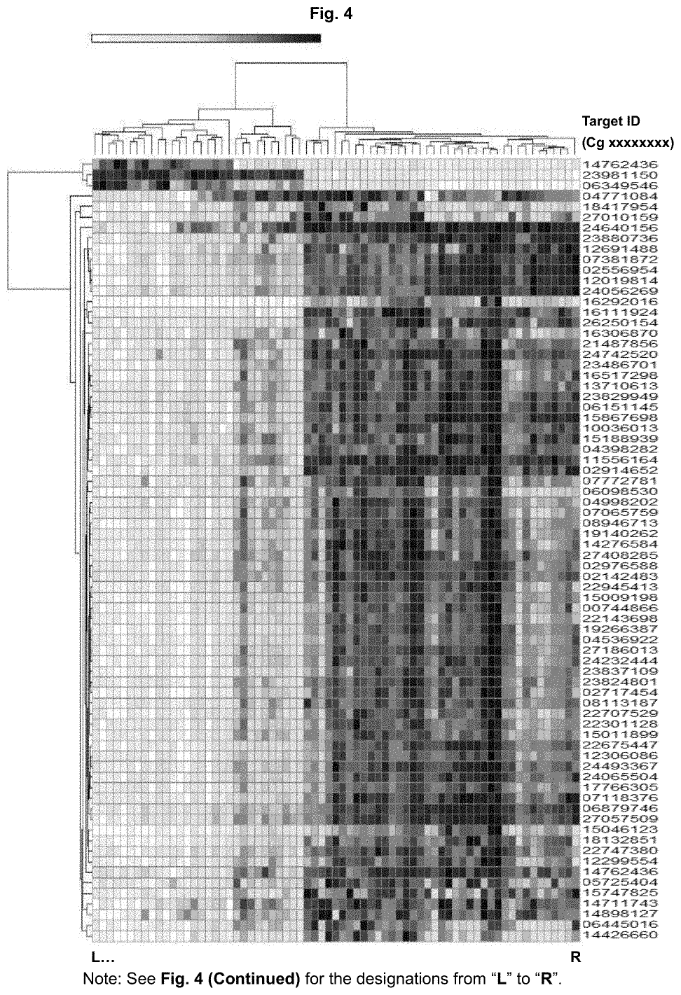

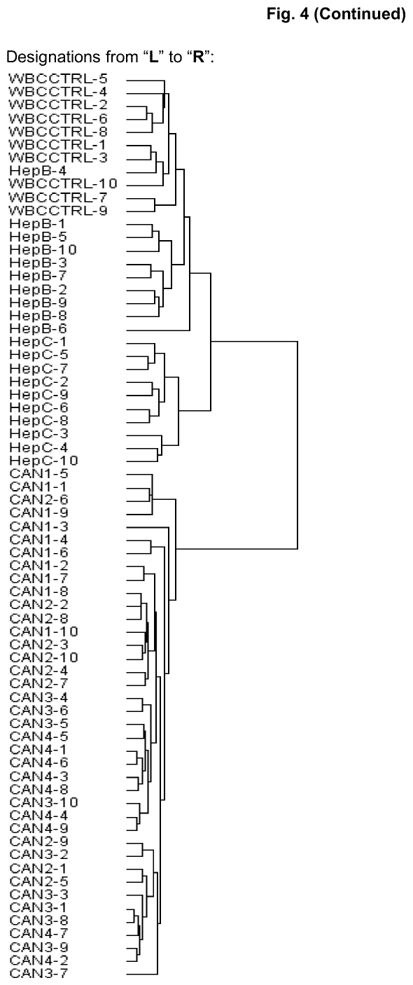

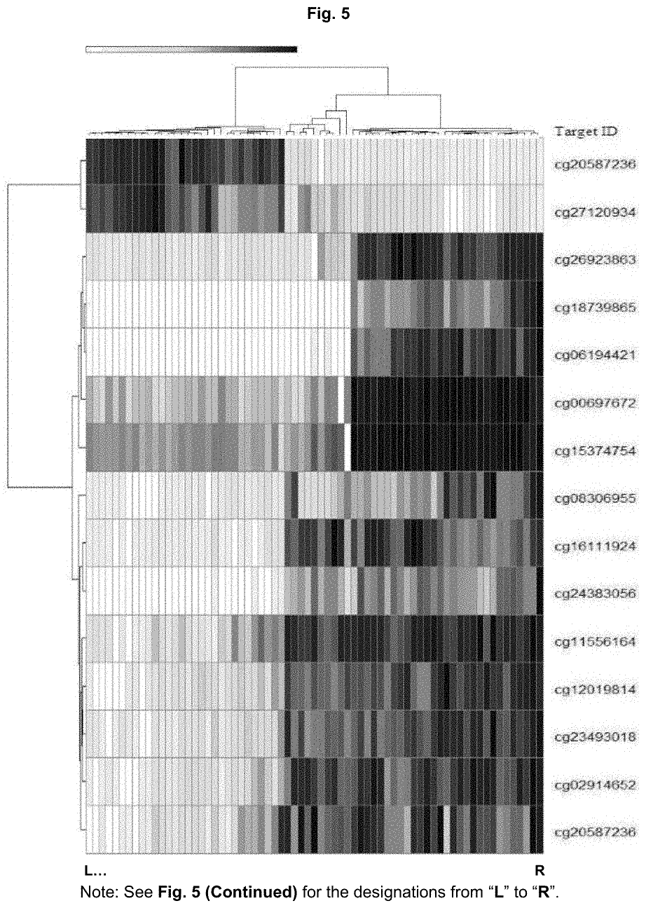

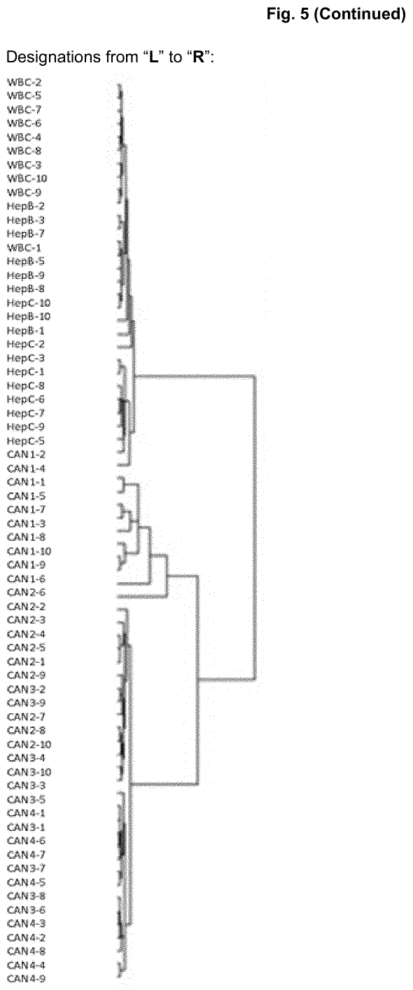

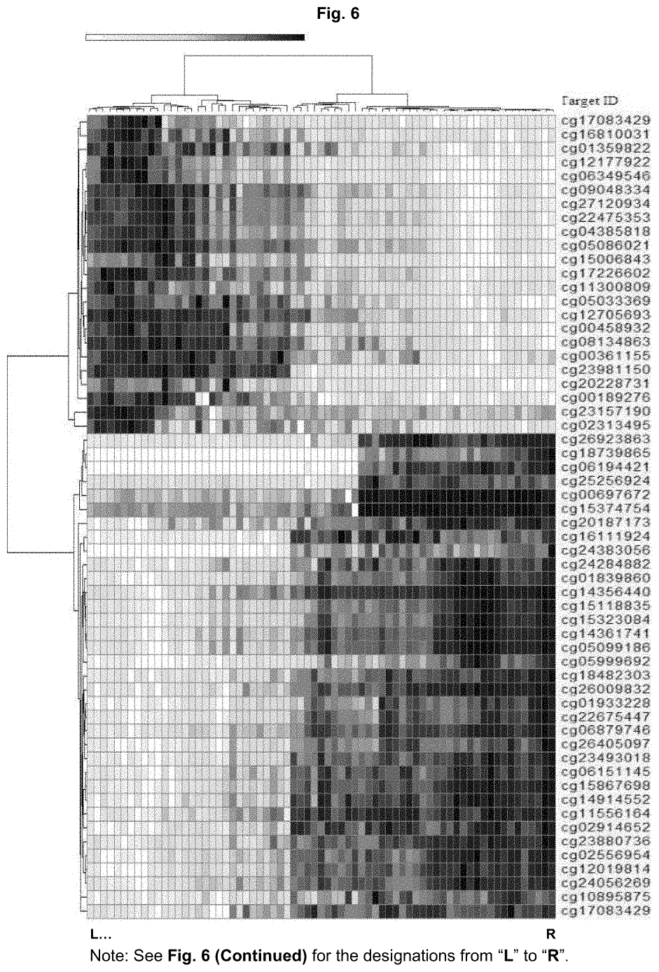

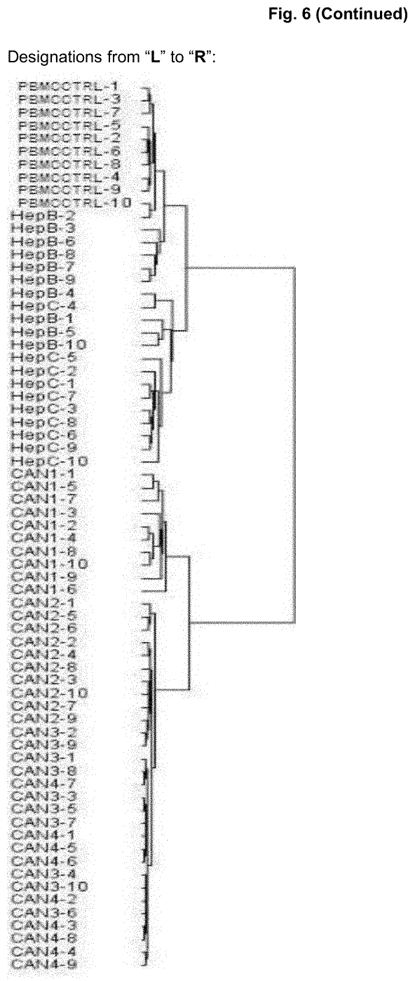

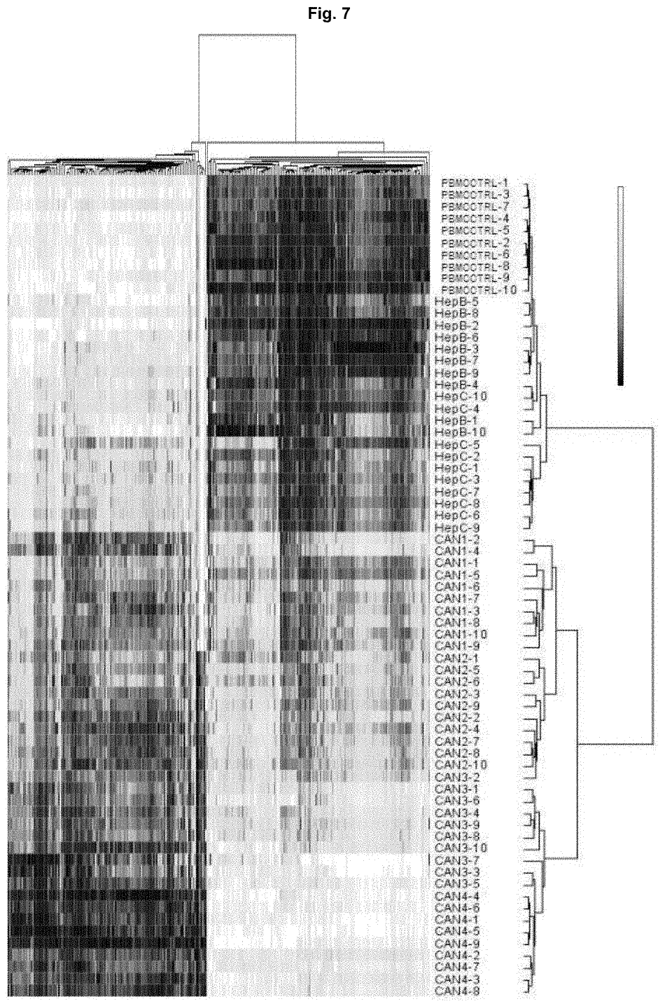

Hierarchical clustering is performed by one minus Pearson correlation for all HCC and hepatitis samples using for each individual analysis a set of CG methylation markers that were “discovered” by testing only one stage of HCC and controls. All other stages were “naïve” to these markers and served as “cross-validation”. Cross validation refers to a statistical strategy whereby a small subset of samples in the study is used to “discover” a list of markers (predictors) that differentiate two groups from each other (i.e., “cancer” and “control”). These “discovered” markers are then tested as predictors in other “new” samples in the study. As demonstrated in FIGS. 4 to 7, each of the independently-derived set of markers for specific stages of HCC were “cross-validated”; they correctly predicted HCC in a group of samples that included “new” HCC and non-HCC cases (FIG. 4 uses stage 1 markers, FIG. 5 uses stage 2 markers, FIG. 6 uses stage 3 markers and FIG. 7 uses stage 4 markers). Remarkably, the CG markers that were discovered by just comparing only one stage of HCC to healthy controls correctly predicted HCC in a different set of samples that included HCC and chronic hepatitis cases. This provides further evidence for a different DNA methylation profile for chronic hepatitis and cancer that could be utilized for predicting whether a patient has still chronic hepatitis or whether he/she has transitioned into HCC. Interestingly, the same markers predicted correctly Hepatitis B and C cases as well (FIGS. 4-7).

The overlap between independently derived CG markers that differentiate each of the HCC stages (FIG. 3A) is significant for all possible overlaps between the stages using Fisher hypergeometric test (p<1.921718e⁻²⁹⁷). The highly significant overlap between the markers derived for each stage independently using only 10 cases and controls strongly validates the robustness of these markers and illustrates the utility of these differentially methylated CGs as peripheral markers of HCC that could be used for early detection.

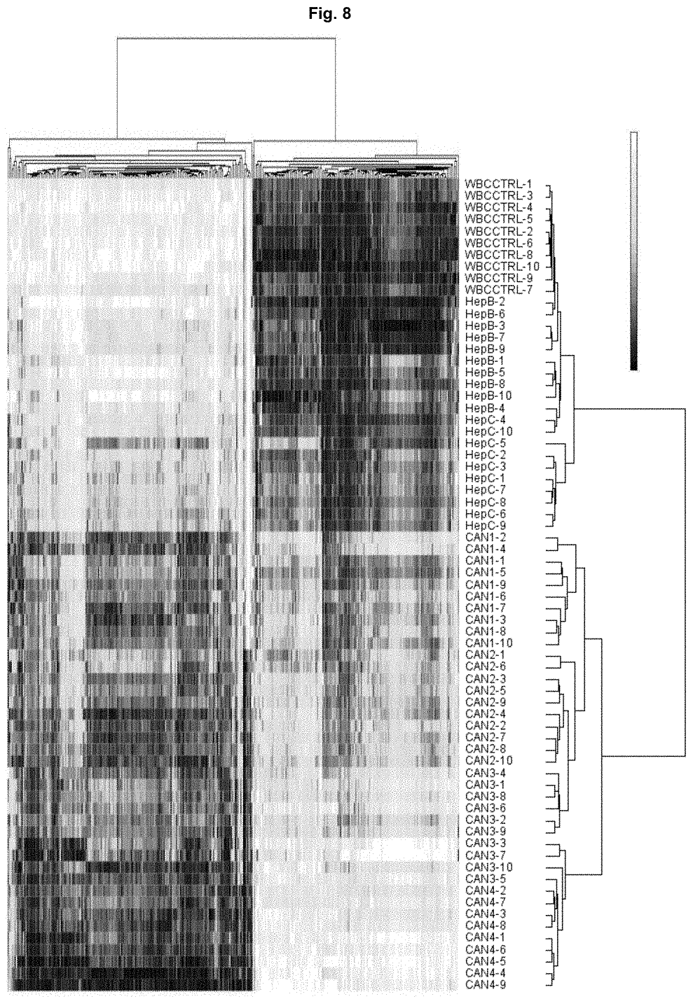

Although there is a large overlap between CGs that are differentially methylated at the different stages of cancer, the overlap is partial. These studies demonstrate that one could utilize the 350 CG list (described above) (Table 3) to differentiate HCC stages from each other. Hierarchical clustering by one minus Pearson correlation of all samples using these 350 CGs correctly clustered the HCC cases by stage while hepatitis B and C cases were clustered with healthy controls. Although there is a large overlap between sites that are differentially methylated from healthy controls at different stages of HCC, the intensity of differential methylation is enhanced with progression of HCC. Thus, the level of methylation of these 350 CG sites could be also used to differentiate stages of HCC. A kit, comprising means and reagents for detecting DNA methylation measurements of the CG IDs of table 3, could be used for predicting hepatocellular carcinoma (HCC) stages and chronic hepatitis. Note that the DNA methylation markers list was derived by comparing only healthy controls and single stages of HCC, nevertheless this list could correctly predict other “new” hepatitis B and C cases as non-HCC (FIG. 8).

These studies disclosed herein reveal differentially methylated CGs in PBMC from HCC patients that can be used to distinguish particular stages of HCC from controls and from chronic hepatitis patients.

### Embodiment 4. Stage Specific CG Methylation Markers That Differentiate Early from Late Stages of HCC Using Penalized Regression

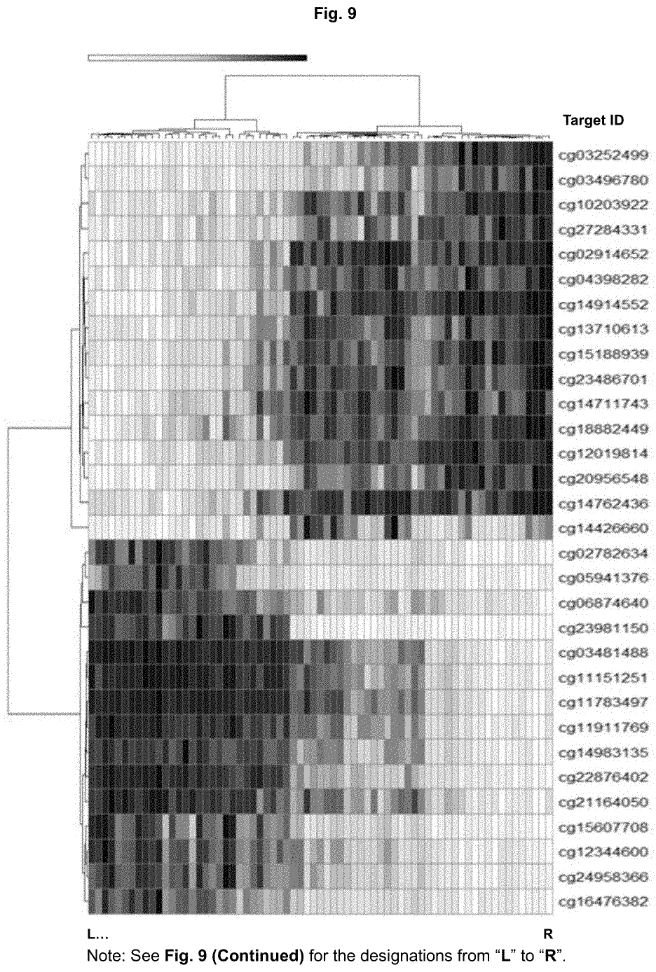

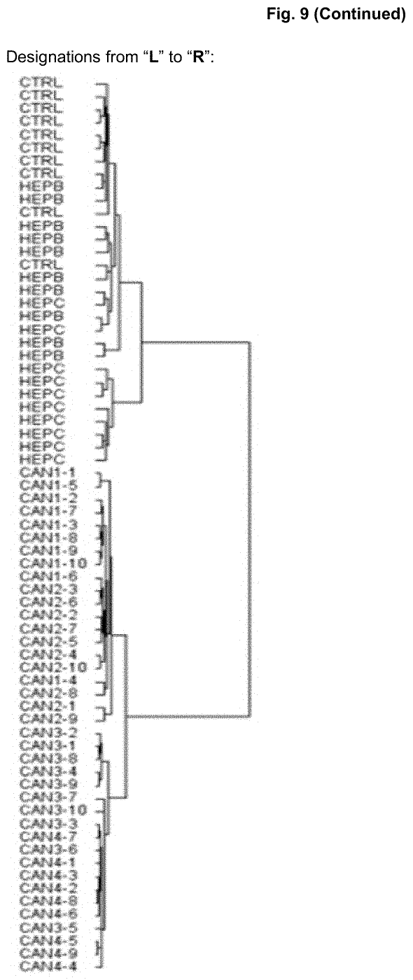

Data suggest that PBMC DNA methylation markers differentiate stages of HCC. This study defines a list of the minimal number of CG sites that are required to differentiate stages of HCC from each other. “Penalized regression” of the 350 CG sites is performed between stage samples using the R package “penalized” for fitting penalized regression models (51). The penalized R package uses likelihood cross-validation and predictions are made on each left-out subject. The fitted model identified 8 CGs that predict stage 1 versus control, 5CGs that predict stage 2 versus control, 5 CGs that differentiate stage 3 versus control, 7 CGs that differentiate Stage 4 versus control and 7 CGs that are sufficient to differentiate stage 1 from hepatitis B (Table 4). 8 CGs are selected that differentiate between stage 1 and later stages 2-4, 10CGs that differentiate stage 1 and 2 from later stages 3-4 and 7 CGs that differentiate stage 4 from all earlier stages (stages 1-3) (Table 4). DNA methylation measurements in PBMC of the combined list of 31 CG stage-separators (after removing duplicates, table 5) accurately predicted all HCC cases and their stages using One minus Pearson clustering (FIG. 9). A kit, comprising means and reagents for detecting DNA methylation measurements of the CG IDs of table 4 or 5, could be used for predicting hepatocellular carcinoma (HCC) stages.

TABLE 4
CG markers differentiating different stages of HCC from control
and hepatitis B and C using penalized regression models.

| | |
|---|---|
| Target CG IDs for | cg14983135, cg10203922, cg05941376, cg14762436, cg12019814, |
| separating HCC stage 1 | cg14426660, cg18882449, cg02914652 |
| from controls: | |
| Target CG IDs for | cg05941376, cg15188939, cg12344600, cg03496780, cg12019814 |
| separating HCC stage 2 | |
| from controls: | |
| Target CG IDs for | cg05941376, cg02782634, cg27284331, cg12019814, cg23981150 |
| separating HCC stage 3 | |
| from controls: | |
| Target CG IDs for | cg02782634, cg05941376, cg10203922, cg12019814, cg14914552, |
| separating HCC stage 4 | cg21164050, cg23981150 |
| from controls: | |
| Target CG IDs for | cg05941376, cg10203922, cg11767757, cg04398282, cg11151251, |
| separating HCC stage 1 | cg24742520, cg14711743 |
| from hepatitis B: | |
| Target CG IDs for | cg03252499, cg03481488, cg04398282, cg10203922, cg11783497, |
| separating HCC stage 1 | cg13710613, cg14762436, cg23486701 |
| from stage 2-4: | |
| Target CG IDs for | cg02914652, cg03252499, cg11783497, cg11911769, cg12019814, |
| separating HCC stage 2 | cg14711743, cg15607708, cg20956548, cg22876402, cg24958366 |
| from stage 3-4: | |
| Target CG IDs for | cg02782634, cg11151251, cg24958366, cg06874640, cg27284331, |
| separating HCC stage 1-3 | cg16476382, cg14711743 |
| from stage 4: | |

TABLE 5
Combined list of 31 CGs differentiating different stages
of HCC from control and hepatitis B and C using penalized
regression models. (after of removing the duplicated CGs)

| | | | | |
|---|---|---|---|---|
| cg14983135 | cg10203922 | cg05941376 | cg14762436 | cg12019814 |
| cg03496780 | cg02782634 | cg27284331 | cg23981150 | cg14914552 |
| cg13710613 | cg23486701 | cg11911769 | cg14711743 | cg15607708 |
| cg14426660 | cg18882449 | cg02914652 | cg15188939 | cg12344600 |
| cg21164050 | cg03252499 | cg03481488 | cg04398282 | cg11783497 |
| cg20956548 | cg22876402 | cg24958366 | cg11151251 | cg06874640 |
| cg16476382 | | | | |

### Embodiment 5. Utility of the CG Penalized Regression Model to Predict Unknown Samples as Different Stage Cancer with 100% Specificity and Sensitivity

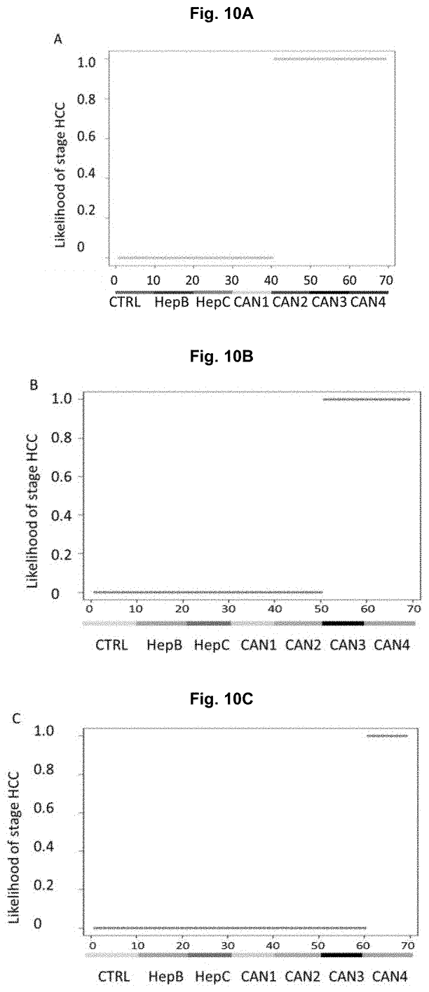

The penalized models derived for differentiating the specific stages using CGs listed in Table 4 were then used on other “naïve” (new samples that were not used for the discovery of the markers) HCC cases and hepatitis B and C controls to predict likelihood of each case being at different stages of HCC. The results of these analyses are shown in FIGS. 10A-10C. The penalized models predicted all the stages samples with 100% sensitivity and 100% specificity.

### Embodiment 6. DNA Methylation Markers that Differentiate Between HCC and Healthy Controls using DNA Extracted from T Cells

Multivariate analysis suggests that the differences in PBMC DNA methylation between HCC and other groups (control and chronic hepatitis) remain even when differences in cell count are taken into account. Further, to determine whether differences in DNA methylation between cancer and control would disappear once the complexity of cell composition is reduced by isolation of a specific cell type (although heterogeneity in T cell subtypes remains), the differences in DNA methylation profiles between T cells isolated from 10 of the 39 HCC patients included in the study (samples from each of the HCC stages, indicated in the legend to table 1) and all healthy controls (n=10) were analyzed to determine whether differences in DNA methylation between cancer and control would disappear once the complexity of cell composition is partly reduced by isolation of a specific cell type.

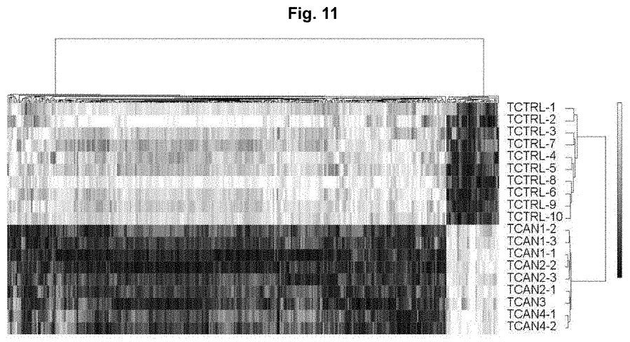

T cells were isolated using antiCD3 immuno-magnetic beads (Dynabed Life technologies), Linear (mixed effects) regression using the ChAMP package on normalized DNA methylation values between HCC and healthy controls revealed 24863 differentially methylated sites at a threshold of p<1×10⁻⁷. 370 robust differentially methylated CGs are shortlisted at a threshold of p<1×10⁻⁷ and delta beta >0.3, <−0.3 (Table 6) and hierarchical clustering of the healthy control and HCC T cell DNA by One minus Pearson correlation was performed (FIG. 11). These 370 CGs correctly cluster all samples into two groups: HCC and controls. A kit, comprising means and reagents for detecting DNA methylation measurements of the CG IDs of table 3, could be used for predicting hepatocellular carcinoma (HCC) stages and chronic hepatitis.

TABLE 6
List of top significant 370 CG IDs derived from T cells
that differentiate HCC from healthy control in cell DNA.

| | | | | | |
|---|---|---|---|---|---|
| cg00014638 | cg02015053 | cg03568507 | cg06098530 | cg08313420 | cg10918327 |
| cg00052964 | cg02086310 | cg03692651 | cg06168204 | cg08479516 | cg10923662 |
| cg00167275 | cg02132714 | cg03764364 | cg06279274 | cg08566455 | cg11065621 |
| cg00168785 | cg02142483 | cg03853208 | cg06445016 | cg08641990 | cg11080540 |
| cg00257775 | cg02152108 | cg03894796 | cg06477663 | cg08644463 | cg11157127 |
| cg00399683 | cg02193146 | cg03909800 | cg06488150 | cg08826152 | cg11231949 |
| cg00404641 | cg02314201 | cg03911306 | cg06568880 | cg08946713 | cg11262262 |
| cg00431894 | cg02322400 | cg03942932 | cg06652329 | cg09122035 | cg11556164 |
| cg00434461 | cg02490460 | cg03976645 | cg06816239 | cg09259081 | cg11692124 |
| cg00452133 | cg02536838 | cg04083575 | cg06822816 | cg09324669 | cg11706775 |
| cg00500229 | cg02556954 | cg04116354 | cg06850005 | cg09555124 | cg11718162 |
| cg00674365 | cg02710015 | cg04192168 | cg06895913 | cg09639931 | cg11909467 |
| cg00772991 | cg02717454 | cg04398282 | cg07019386 | cg09681977 | cg11955727 |
| cg00804338 | cg02750262 | cg04536922 | cg07052063 | cg09696535 | cg11958644 |
| cg00815832 | cg02849693 | cg04656070 | cg07065759 | cg09750084 | cg12019814 |
| cg00898013 | cg02863594 | cg04771084 | cg07145988 | cg10036013 | cg12099423 |
| cg01044293 | cg02914652 | cg04864807 | cg07249730 | cg10061361 | cg12161228 |
| cg01116137 | cg02939781 | cg04998202 | cg07266910 | cg10091662 | cg12299554 |
| cg01124132 | cg02976588 | cg05084827 | cg07381872 | cg10167378 | cg12315391 |
| cg01254303 | cg02991085 | cg05107535 | cg07385778 | cg10184328 | cg12427303 |
| cg01305421 | cg03035849 | cg05132077 | cg07721852 | cg10185424 | cg12549858 |
| cg01359822 | cg03151810 | cg05157625 | cg07772781 | cg10196532 | cg12583076 |
| cg01366985 | cg03204322 | cg05217983 | cg07834396 | cg10274682 | cg12649038 |
| cg01405107 | cg03215181 | cg05304366 | cg07850527 | cg10341310 | cg12691488 |
| cg01413790 | cg03400131 | cg05348875 | cg07912766 | cg10530883 | cg12727605 |
| cg01557792 | cg03441844 | cg05429448 | cg08038033 | cg10549831 | cg12777448 |
| cg01832672 | cg03461110 | cg05460226 | cg08113187 | cg10555744 | cg12789173 |
| cg01921773 | cg03541331 | cg05512157 | cg08123444 | cg10584024 | cg12856392 |
| cg01927745 | cg03544320 | cg05554346 | cg08280368 | cg10890302 | cg12868738 |
| cg01992590 | cg03546163 | cg05759347 | cg08306955 | cg10909506 | cg12880685 |
| cg12906381 | cg15009198 | cg17335387 | cg19795616 | cg22404498 | cg24919348 |
| cg12963656 | cg15011899 | cg17372657 | cg19841369 | cg22589728 | cg25100962 |
| cg12970155 | cg15046123 | cg17597631 | cg19930116 | cg22656550 | cg25104397 |
| cg13260278 | cg15109018 | cg17718703 | cg19988492 | cg22668906 | cg25174412 |
| cg13286116 | cg15145341 | cg17741339 | cg20197130 | cg22675447 | cg25188006 |
| cg13308137 | cg15302376 | cg17765025 | cg20222519 | cg22747380 | cg25310233 |
| cg13401703 | cg15331834 | cg17766305 | cg20478129 | cg22945413 | cg25353287 |
| cg13404054 | cg15514380 | cg17775490 | cg20585841 | cg23299919 | cg25459280 |
| cg13405775 | cg15514896 | cg17786894 | cg20587236 | cg23486701 | cg25461186 |
| cg13435137 | cg15598244 | cg17837517 | cg20606062 | cg23771949 | cg25502144 |
| cg13466988 | cg15695738 | cg17988310 | cg20625523 | cg23824902 | cg25673720 |
| cg13679714 | cg15704219 | cg18031596 | cg20769177 | cg23829949 | cg25779483 |
| cg13896699 | cg15720112 | cg18051353 | cg20781967 | cg23880736 | cg25784220 |
| cg13904970 | cg15747825 | cg18128914 | cg20995304 | cg23944804 | cg25891647 |
| cg13912027 | cg15756407 | cg18132851 | cg21092324 | cg24056269 | cg25964728 |
| cg13939291 | cg15867698 | cg18182216 | cg21222426 | cg24065504 | cg26015683 |
| cg14140403 | cg16111924 | cg18214661 | cg21226442 | cg24070198 | cg26250154 |
| cg14242995 | cg16218221 | cg18273840 | cg21358380 | cg24142603 | cg26325335 |
| cg14276584 | cg16259904 | cg18297196 | cg21384492 | cg24169486 | cg26402555 |
| cg14326196 | cg16292016 | cg18370682 | cg21386573 | cg24232444 | cg26405097 |
| cg14362178 | cg16306870 | cg18417954 | cg21487856 | cg24383056 | cg26407558 |
| cg14376836 | cg16496269 | cg18766900 | cg21816330 | cg24405716 | cg26465602 |
| cg14419424 | cg16512390 | cg18804667 | cg21833076 | cg24453118 | cg26475911 |
| cg14734614 | cg16763089 | cg18808261 | cg21918548 | cg24536818 | cg26594335 |
| cg14762436 | cg16810031 | cg19095568 | cg22088248 | cg24616553 | cg26803268 |
| cg14774438 | cg16894855 | cg19140262 | cg22143698 | cg24631428 | cg26827373 |
| cg14858267 | cg16924102 | cg19193595 | cg22256433 | cg24680439 | cg26856443 |
| cg14898127 | cg17144149 | cg19266387 | cg22301128 | cg24716416 | cg26876834 |
| cg14914552 | cg17173975 | cg19760965 | cg22303909 | cg24729928 | cg26963367 |
| cg15000827 | cg17221813 | cg19768229 | cg22374742 | cg24742520 | cg27010159 |
| cg27098685 | cg27113419 | cg27186013 | cg27207470 | cg27247736 | cg27300829 |
| cg27406664 | cg27408285 | cg27544294 | cg27576694 | | |

### Embodiment 7. Utility of DNA Methylation Marker Discovered in T cells to Predict “Untrained” HCC and Chronic Hepatitis Patients

These 370 CG sites that differentiate T cells from HCC and healthy controls (Table 6) could be used to cluster “untrained” different chronic hepatitis and healthy control PBMC samples (n=69).

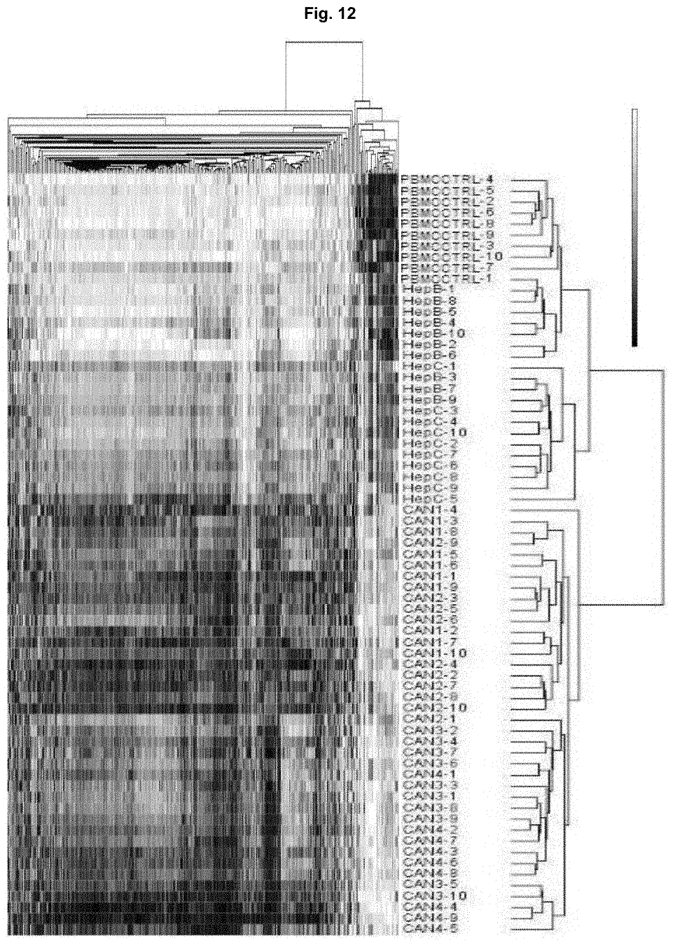

The clustering analysis presented in FIG. 12 shows that the 370 CG sites that are differentially methylated in T cells DNA cluster individual HCC, hepatitis and healthy control DNA from PBMC with 100% accuracy. Thus, the differentially methylated CGs discovered using T cell DNA were “cross validated” on different patients (29 different patients with HCC, and 20 with chronic hepatitis) using DNA methylation measurements in PBMC.

### Embodiment 8. Utility of 350 CG Sites (Table 3) and 31CG Sites (Table 5) Derived from Analysis of PBMC DNA in Predicting HCC Cancer Using T Cell DNA

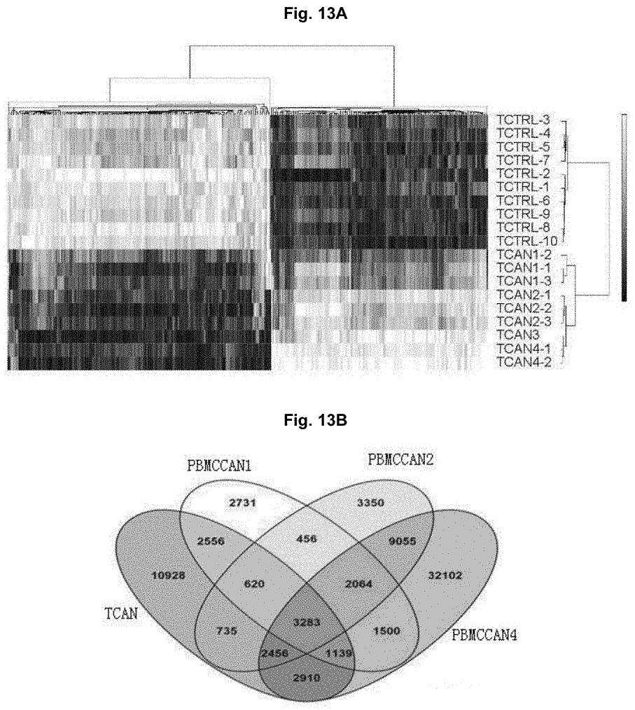

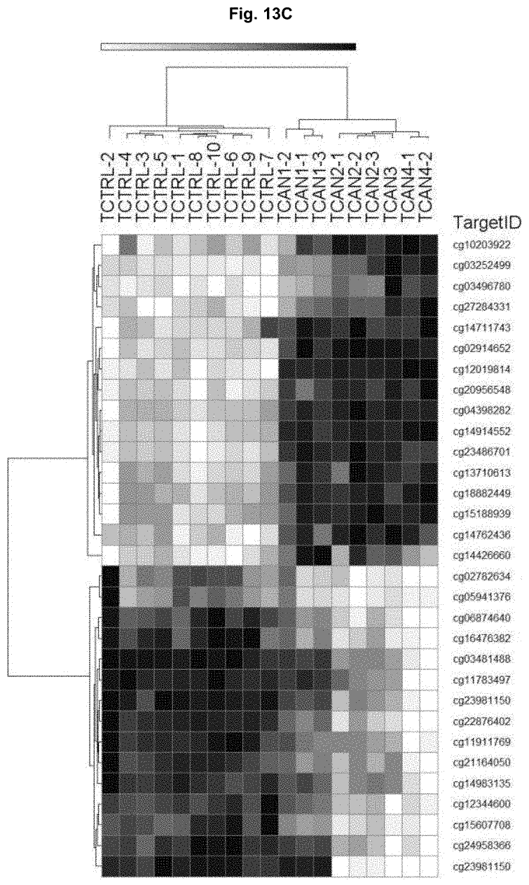

The 350 CGs that were derived by analysis of PBMC DNA clustered the T cell healthy controls and HCC samples correctly (FIG. 13A). There is a highly significant overlap between the significant CGs (Fisher, p<1×10⁻⁷) that differentiate healthy controls from HCC using T cell DNA and CGs that differentiate the different HCC stages and controls using PBMC DNA (FIG. 13B).

The present disclosure also shows that the shortlisted 31 CGs derived by penalized regression from PBMC DNA methylation measures (Table 5) also cluster and stage accurately T cell

DNA methylation measurements from HCC patients and controls using One minus Pearson correlations (FIG. 13C). These data demonstrate that the differences in DNA methylation between HCC and other samples remains even when the complexity of cell types is reduced by isolation of particular cell types and provides further “cross-validation” for the association of these CGs with HCC and their predictive value.

### Embodiment 9. Differentially Methylated Genes in PBMC in HCC are Enriched in Immune Related Canonical Pathways

Progression of HCC has a broad footprint in the methylome (the genome-wide DNA methylation profile) (FIGS. 1A-1B). To gain insight into the functional footprint of the differentially methylated genes in PBMC and T cells from HCC patients, the gene lists generated from the differential methylation analyses were subjected to a gene set enrichment analysis using Ingenuity Pathway Analysis (IPA). Genes associated with CGs were first subjected to gene set enrichment analysis, said CGs show linear correlation with stages of HCC in the Pearson correlation analysis (FIG. 1) (r>0.8; r<−0.8; delta beta>0.2, delta beta<−0.2). Notably the top upstream regulators of genes associated with these CGs are TGFbeta (p<1.09×10⁻¹⁷), TNF (p<7.32×10⁻¹⁵), dexamethasone (p<7.74×10⁻¹²) and estradiol (p<4×10⁻¹²) which are major immune inflammation and stress regulators of the immune system. Top diseases identified were cancer (p value 1×10-5 to 2×10⁻⁵¹) and hepatic disease (p<1.24×10⁻⁵ to 1.11×10⁻²⁵). A strong signal was noted for Liver hyperplasia (p<6.19×10⁻¹ to 1.11×10⁻²⁵) and hepatocellular carcinoma (p<5.2×10⁻¹ to 3.76×10⁻²⁵). An inspection of the genes that are differentially methylated reveals a large representation of immune regulatory molecules such as IL2, IL4, ILS, IL16, IL7, 1110, IL18, 1124, IllB and interleukin receptors such as IL12RB2, IL1B, IL1R1, IL1R2, IL2RA, IL4R, IL5RA; chemokines such as CCL1, CCL7, CCL18, CCL24, as well as chemokine receptors such CCR6, CCR7 and CCR9; cellular receptors such as CD2, CD6, CD14, CD38, CD44, CD80 and CD83; TGFbeta3 and TGFbeta1, NFKB, STAT1, STAT3 and TNFa.

A comparative IPA analysis between PBMC and T cells differentially methylated genes revealed NFKB, TNF, VEGF and IL4 and NFAT as common upstream regulators. Overall, the DNA methylation alterations in HCC PBMC and T cell show a strong signature in immune modulation functions. Differentially methylated promoters between HCC and noncancerous liver tissue were previously delineated (16, 58). The present disclosure also provides a method to determine whether there was an overlap between the promoters that are differentially methylated in HCC in the cancer biopsies (1983 promoters) and peripheral blood mononuclear cells (545 promoters) and found an overlap of 44 promoters which was not statistically significant as determined by Fisher hypergeometric test (p=0.76). These data show that the changes in DNA methylation seen in peripheral blood mononuclear cells reflect changes in the immune system in HCC and that these differentially methylated CGs are most probably not a footprint of circulating DNA from tumors or “surrogates” of DNA methylation changes occurring in the tumor. The utility of these pathways is by providing new targets for cancer therapeutics in the peripheral immune system.

### Embodiment 10. Predicting HCC and Cancer by Pyrosequencing of Differentially Methylated CGs

Pyrosequencing was performed using the PyroMark Q24 machine and results were analyzed with PyroMark® Q24 Software (Qiagen). All data were expressed as mean±standard error of the mean (SEM). The statistical analysis was undertaken using R. Primers used for the analysis are listed in Table 7

.

TABLE 7
Pyrosequencing assays for HCC predictors; AHNAK, SLFN2L, AKAP7, STAP1.
Table 7 discloses SEQ ID NOS 1-20, respectively, in order of appearance.

| Gene | Primers | sequence(5′-----3′) |
|---|---|---|
| AHNAK | out Forward | GGATGTGTCGAGTAGTAGGGT |
| | out Reverse, | CCTATCATCTCCACACTAACGCT |
| | nest Forward | TGTTAGGGGTGATTTTTAGAGG |
| | nest R(biotin) | ATTAACCCCATTTCCATCCTAACTATCTT |
| | sequencing primer | TTTTAGAGGAGTTTTTTTTTTTTA |
| SLFN12L | out Forward | GTGATYTTGGTYAYTGTAAYYT |
| | out Reverse | TCTCATCTTTCCATARACATTTATTTAR |
| | nest Forward | AGGGTTTYAYTATATTAGYYAGGTTGG |
| | nest Reverse (biotin) | ATRCAAACCATRCARCCCTTTTRC |
| | sequencing primer | YYYAAAATAYTGAGATTATAGGTGT |
| AKAP7 | out Forward | TAGGAGAAAGGGTTTATTGTGGT |
| | out Reverse | ACACACCCTACCTTTTTCACTCCA |
| | nest Forward | GGTATTGATTTATGGTTAGGGATTTATAG |
| | nest Reverse(biotin) | AAACAAAAAAAACTCCACCTCCAATCC |
| | sequencing primer | GGGATTTATAGTTTTGTGAGA |
| STAP1 | out Forward | AGTYATGTYTTYTGYAAATAAAAATGGAYAYY |
| | out Reverse | TTRCTTTTTACCACCAACACTACC |
| | nest Forward | YYGTTTYTTTYATYTTYTGGTGATGTTAA |
| | nest Reverse(biotin) | ARARRRCCAATCTCTRRRTAATCCACATRTR |
| | sequencing primer | GGTGATGTTAATYTTYTGTTTA |

For the replication set, this study uses T cells DNA to reduce cell composition issues. The replication set included 79 people, 10 healthy controls and 10 individuals from each of the hepatitis B and C and 3 cancer stages and 19 stage 1 samples (Table 2). Following genes are examined that were found to be significantly differentially methylated in T cells in comparison with HCC in the discovery set: STAP1 (cg04398282) (also included in table 6), AKAP7 (cg12700074), SLFNL2 (cg00974761), and included 1 additional hypomethylated gene in HCC: Neuroblast differentiation-associated protein (AHNAK) (cg14171514). Linear regression between all controls (healthy and hepatitis B and C) and HCC stage 1,2 (0+A) revealed significant association with HCC stage 1,2 for all 4 CGs after correction for multiple testing (STAP1 p=4.04×10⁻⁷; AKAP7 p=0.046; SLFNL2 p=0.012; AHNAK p=0.003436). Linear regression between all controls and all stages of HCC revealed significant association for STAP1 (p=6.6×10⁻⁶) and AHNAK with HCC (p=0.026) after correction for multiple testing.

ANOVA analysis revealed a significant difference in methylation between the control group (healthy controls and hepatitis B and C) and the group of early HCC (stages 0+A; 1,2) in all 4 CGs that were validated. A group comparison between all controls and all HCC revealed a significant difference in methylation for STAP1 (p=1.7×10⁻⁶), AKAP7 (p=0.042), AHNAK (p=0.0062) but the difference for SLFNL2 was trendy but not significant (p=0.071). ANOVA revealed significant effect for diagnosis (F=10.017; p=7.49×10⁻⁶) on STAP1 methylation.

Pairwise analysis after correction for multiple testing on the 5 different diagnosis subgroups of controls (healthy controls, chronic hepatitis B and chronic hepatitis C) and early HCC (stages 1 and 2 or 0 and A) revealed significant differences between stage 1 (BCLC 0) HCC and either healthy controls (p=0.00037), chronic hepatitis B (p=0.00849) or hepatitis C (p=0.00698) and between stage 2 (BCLC A) and either healthy controls (p=0.00018), hepatitis B (p=0.00670) or hepatitis C (p=0.00534). While there was also an effect of diagnosis on SLFN2L methylation (F=3.9376; p=0.00810) AHNAK (F=3.0219; p=0.02809) and AKAP7 (F=3.4; p=0.01633), pairwise comparisons between the different diagnosis subgroups were not significant.

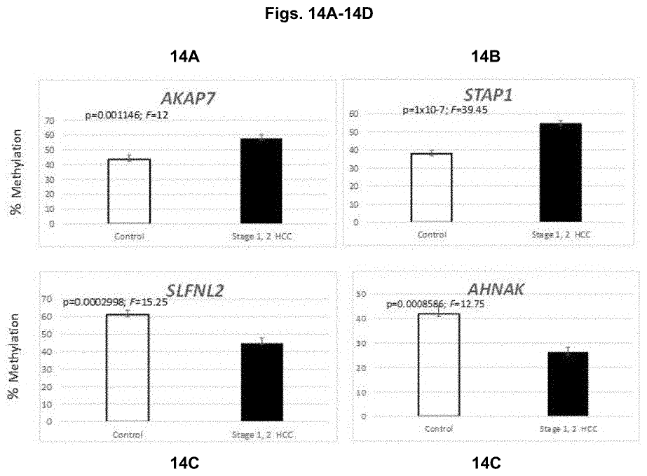

These data illustrates that these 4 CG sites could be used to predict early stages of HCC and differentiate them from controls (FIGS. 14A-14D).

### Embodiment 11. Utility of the Discovered List of Differentially Methylated CGs to Predict HCC by Receiver Operating Characteristic (ROC) Analysis; the Example of STAP1

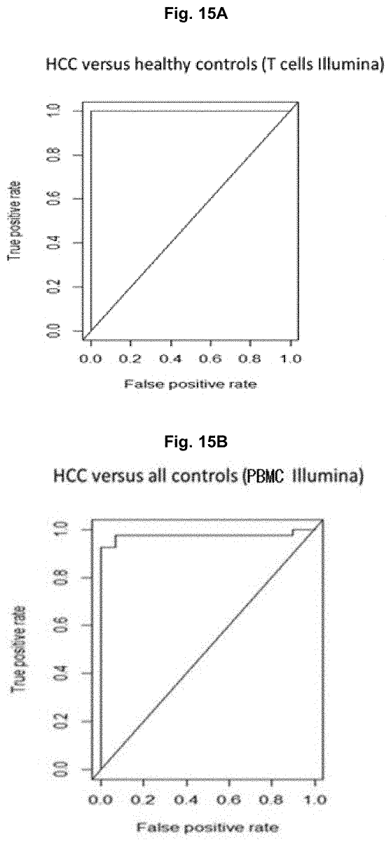

A measure of the diagnostic value of a biomarker is the Receiver Operating Characteristic (ROC) which measures “sensitivity” (fraction of true discoveries) as a function of “specificity” (fraction of false discoveries). The ROC test determines a threshold value (ie. percentage of methylation at a particular CG) that provides the most accurate prediction (the highest fraction of “true discoveries” and the least number of “false discoveries”) (59) (FIGS. 15A-15B). The DNA methylation level of each sample is compared to a threshold DNA methylation value and is then classified as either control or HCC. The present disclosure provides for the first time that determines ROC characteristics for the normalized Illumina 450K beta values for T cells from healthy controls and HCC (FIG. 15A). The STAP1 gene cg04398282 behaves as a perfect biomarker. With a threshold DNA methylation beta value of 0.757 (any sample that has higher value is classified as HCC and lower value than 0.757 as control) the accuracy for calling HCC samples was 100%, the AUC is 1 and both sensitivity and specificity are 100%. The STAP1 biomarker was discovered by comparing T cells DNA methylation from HCC and healthy controls. We therefore could cross-validate the biomarker properties of STAP1 cg04398282 by examining the ROC characteristics using normalized beta values from the PBMC DNA samples which included hepatitis B and hepatitis C patients as well as 29 additional HCC patients that were not included in the T cells DNA methylation analysis (FIG. 15B).

The accuracy of predicting all HCC samples (all stages) using PBMC DNA was 96% using a threshold beta value of 0.6729 and the AUC was 0.9741379 (sensitivity 0.975 and specificity 0.973). The ROC characteristics are examined using pyrosequencing values of STAP1 in the replication set of T cell DNA (FIGS. 16A-16B). The CG methylation values of this STAP1 as quantified by pyrosequencing site were overall lower than Illumina 450K values. At threshold of DNA methylation of 40.2% for STAP1 cg04398282, the accuracy of calling HCC from all other controls (healthy and hepatitis B and C) is 82.2%. The area under the curve (AUC) for discrimination between HCC and all controls is: 0.8 (85% sensitivity and 73% specificity) (FIG. 16A). At threshold of 50.12% methylation of STAP1 cg04398282 the accuracy of calling HCC stage 1 from all controls is 83.6% and the AUC is 0.89 (84% sensitivity and 83% specificity). The accuracy of differentiating HCC stage 1 from healthy controls (FIG. 16A) is 93% at a threshold methylation level of 47.2 and the AUC is 0.94 (94% sensitivity and 94% specificity) (FIG. 16B). In summary, STAP1 illustrates that DNA methylation biomarkers in HCC peripheral blood mononuclear cells could be used for discriminating Stage 1 from chronic hepatitis and healthy controls which is a critical hurdle in early diagnosis of liver cancer. STAP1 was identified using T cell DNA and was validated in the replication set (FIGS. 14A-14D).

The methods used here to measure DNA methylation provide only an example and do not exclude measurements of DNA methylation by other acceptable methods. It should be noted that any person skilled in the art could measure DNA methylation of STAP1 and other differentially methylated sites using a number of accepted and available methods that are well documented in the public domain including for example, Illumina 850K arrays, mass spectrometry based methods such as Epityper (Seqenom), PCR amplification using methylation specific primers (MS-PCR), high resolution melting (HRM), DNA methylation sensitive restriction enzymes and bisulfite sequencing.

### Applications of the Disclosure

The applications of the disclosure are in the field of molecular diagnostics of HCC and cancer in general. Any person skilled in the art could use this diagnostic method to derive similar biomarkers for other cancers. Moreover, the genes and the pathways derived from the genes can guide new drugs that focus on the peripheral immune system using the targets listed in embodiment 9. The focus in DNA methylation studies in cancer to date has been on the tumor, tumor microenvironment (8, 9) and circulating tumor DNA (5, 6) and major advances were made in this respect. However, the question remains of whether there are DNA methylation changes in host systems that could instruct us on the system wide mechanisms of the disease and/or serve as noninvasive predictors of cancer. HCC is a very interesting example since it frequently progresses from preexisting chronic hepatitis and liver cirrhosis (2) and could provide a tractable clinical paradigm for addressing this question. This present disclosure provides that the qualities of the host immune system might define the clinical emergence and trajectory of cancer.

Importantly, the present disclosure shows a sharp boundary between stage 1 of HCC and chronic hepatitis B and C that could be used to diagnose early transition from chronic hepatitis to HCC as illustrated in the embodiments of present disclosure. The present disclosure also provides how this diagnosis could be used to separate stages of cancer from each other. All assays require a set of known samples with methylation values for the CG IDs disclosed in the present disclosure to train the models using hierarchical clustering, ROC or penalized regression and unknown samples will then be analyzed using these models as illustrated in the embodiments of the present disclosure.

The fact that the present disclosure is mentioning different dependent claims does not mean that one cannot use a combination of these claims for predicting cancer. The examples disclosed here for measuring and statistically analyzing and predicting cancer, stages of cancer and chronic hepatitis should not be considered limiting. Various other modifications will be apparent to those skilled in the art to measure DNA methylation in cancer patients such as Illumina 850K arrays, capture array sequencing, next generation sequencing, methylation specific PCR, epityper, restriction enzyme based analyses and other methods found in the public domain. Similarly, there are numerous statistical methods in the public domain in addition to those listed here to use for prediction of cancer in patient samples.

### REFERENCES

1. El-Serag H B. Hepatocellular carcinoma. N Engl J Med. 2011; 365:1118-27.
2. Flores A, Marrero J A. Emerging trends in hepatocellular carcinoma: focus on diagnosis and therapeutics. Clinical Medicine Insights Oncology. 2014; 8:71-6.
3. Tan C H, Low S C, Thng C H. APASL and AASLD Consensus Guidelines on Imaging Diagnosis of Hepatocellular Carcinoma: A Review. International journal of hepatology. 2011; 2011:519783.
4. Valente S, Liu Y, Schnekenburger M, Zwergel C, Cosconati S, Gros C, et al. Selective non-nucleoside inhibitors of human DNA methyltransferases active in cancer including in cancer stem cells. J Med Chem. 2014; 57:701-13.
5. Jiao L, Zhu J, Hassan M M, Evans D B, Abbruzzese J L, Li D. K-ras mutation and p16 and preproenkephalin promoter hypermethylation in plasma DNA of pancreatic cancer patients: in relation to cigarette smoking. Pancreas. 2007; 34:55-62.
6. Park J W, Baek I H, Kim Y T. Preliminary study analyzing the methylated genes in the plasma of patients with pancreatic cancer. Scand J Surg. 2012; 101:38-44.
7. Dirix L, Van Dam P, Vermeulen P. Genomics and circulating tumor cells: promising tools for choosing and monitoring adjuvant therapy in patients with early breast cancer? Curr Opin Oncol. 2005; 17:551-8.
8. Finak G, Laferriere J, Hallett M, Park M. [The tumor microenvironment: a new tool to predict breast cancer outcome]. Med Sci (Paris). 2009; 25:439-41.
9. Finak G, Sadekova S, Pepin F, Hallett M, Meterissian S, Halwani F, et al. Gene expression signatures of morphologically normal breast tissue identify basal-like tumors. Breast Cancer Res. 2006; 8:R58.
10. Sehouli J, Loddenkemper C, Cornu T, Schwachula T, Hoffmuller U, Grutzkau A, et al. Epigenetic quantification of tumor-infiltrating T-lymphocytes. Epigenetics. 2011; 6:236-46.
11. Jeschke J, Collignon E, Fuks F. DNA methylome profiling beyond promoters: taking an epigenetic snapshot of the breast tumor microenvironment. FEBS J. 2014.
12. Baylin S B, Esteller M, Rountree M R, Bachman K E, Schuebel K, Herman J G. Aberrant patterns of DNA methylation, chromatin formation and gene expression in cancer. Hum Mol Genet. 2001; 10:687-92.
13. Issa J P, Vertino P M, Wu J, Sazawal S, Celano P, Nelkin B D, et al. Increased cytosine DNA-methyltransferase activity during colon cancer progression. J Natl Cancer Inst. 1993; 85:1235-40.
14. Ehrlich M. DNA methylation in cancer: too much, but also too little. Oncogene. 2002; 21:5400-13.
15. Aguirre-Ghiso J A. Models, mechanisms and clinical evidence for cancer dormancy. Nat Rev Cancer. 2007; 7:834-46.
16. Stefanska B, Huang J, Bhattacharyya B, Suderman M, Hallett M, Han Z G, et al. Definition of the landscape of promoter DNA hypomethylation in liver cancer. Cancer Res. 2011; 71:5891-903.
17. Stefansson O A, Moran S, Gomez A, Sayols S, Arribas-Jorba C, Sandoval J, et al. A DNA methylation-based definition of biologically distinct breast cancer subtypes. Mol Oncol. 2014.
18. Radpour R, Barekati Z, Kohler C, Lv Q, Burki N, Diesch C, et al. Hypermethylation of tumor suppressor genes involved in critical regulatory pathways for developing a blood-based test in breast cancer. PLoS One. 2011; 6:e16080.
19. Ramzy, I I, Omran D A, Hamad O, Shaker O, Abboud A. Evaluation of serum LINE-1 hypomethylation as a prognostic marker for hepatocellular carcinoma. Arab journal of gastroenterology: the official publication of the Pan-Arab Association of Gastroenterology. 2011; 12:139-42.
20. Chan K C, Jiang P, Chan C W, Sun K, Wong J, Hui E P, et al. Noninvasive detection of cancer-associated genome-wide hypomethylation and copy number aberrations by plasma DNA bisulfite sequencing. Proc Natl Acad Sci U S A. 2013; 110:18761-8.
21. Blair G E, Cook G P. Cancer and the immune system: an overview. Oncogene. 2008; 27:5868.
22. Ehrlich P. Ueber den jetzigen Stand der Karzinomforschung. Ned Tijdschr Geneeskd. 1909; 5:273-90.
23. Vesely M D, Kershaw M H, Schreiber R D, Smyth M J. Natural innate and adaptive immunity to cancer. Annual review of immunology. 2011; 29:235-71.
24. Dunn G P, Bruce A T, Ikeda H, Old L J, Schreiber R D. Cancer immunoediting: from immunosurveillance to tumor escape. Nature immunology. 2002; 3:991-8.
25. Swann J B, Smyth M J. Immune surveillance of tumors. The Journal of clinical investigation. 2007; 117:1137-46.
26. Mackensen A, Ferradini L, Carcelain G, Triebel F, Faure F, Viel S, et al. Evidence for in situ amplification of cytotoxic T-lymphocytes with antitumor activity in a human regressive melanoma. Cancer research. 1993; 53:3569-73.
27. Ferradini L, Mackensen A, Genevee C, Bosq J, Duvillard P, Avril M F, et al. Analysis of T cell receptor variability in tumor-infiltrating lymphocytes from a human regressive melanoma. Evidence for in situ T cell clonal expansion. The Journal of clinical investigation. 1993; 91:1183-90.
28. Zorn E, Hercend T. A natural cytotoxic T cell response in a spontaneously regressing human melanoma targets a neoantigen resulting from a somatic point mutation. European journal of immunology. 1999; 29:592-601.
29. Zorn E, Hercend T. A MAGE-6-encoded peptide is recognized by expanded lymphocytes infiltrating a spontaneously regressing human primary melanoma lesion. European journal of immunology. 1999; 29:602-7.
30. Carcelain G, Rouas-Freiss N, Zorn E, Chung-Scott V, Viel S, Faure F, et al. In situ T-cell responses in a primary regressive melanoma and subsequent metastases: a comparative analysis. International journal of cancer Journal international du cancer. 1997; 72:241-7.
31. Knuth A, Danowski B, Oettgen H F, Old L J. T-cell-mediated cytotoxicity against autologous malignant melanoma: analysis with interleukin 2-dependent T-cell cultures. Proceedings of the National Academy of Sciences of the United States of America. 1984; 81:3511-5.
32. Schumacher K, Haensch W, Roefzaad C, Schlag P M. Prognostic significance of activated CD8(+) T cell infiltrations within esophageal carcinomas. Cancer research. 2001; 61:3932-6.
33. Conejo-Garcia J R, Benencia F, Courreges M C, Gimotty P A, Khang E, Buckanovich R J, et al. Ovarian carcinoma expresses the NKG2D ligand Letal and promotes the survival and expansion of CD28− antitumor T cells. Cancer research. 2004; 64:2175-82.
34. Sato E, Olson S H, Ahn J, Bundy B, Nishikawa H, Qian F, et al. Intraepithelial CD8+tumor-infiltrating lymphocytes and a high CD8+/regulatory T cell ratio are associated with favorable prognosis in ovarian cancer. Proceedings of the National Academy of Sciences of the United States of America. 2005; 102:18538-43.
35. Naito Y, Saito K, Shiiba K, Ohuchi A, Saigenji K, Nagura H, et al. CD8+ T cells infiltrated within cancer cell nests as a prognostic factor in human colorectal cancer. Cancer research. 1998; 58:3491-4.
36. Galon J, Costes A, Sanchez-Cabo F, Kirilovsky A, Mlecnik B, Lagorce-Pages C, et al. Type, density, and location of immune cells within human colorectal tumors predict clinical outcome. Science. 2006; 313:1960-4.
37. Pages F, Berger A, Camus M, Sanchez-Cabo F, Costes A, Molidor R, et al. Effector memory T cells, early metastasis, and survival in colorectal cancer. The New England journal of medicine. 2005; 353:2654-66.
38. Teng M W, Vesely M D, Duret H, McLaughlin N, Towne J E, Schreiber R D, et al. Opposing roles for IL-23 and IL-12 in maintaining occult cancer in an equilibrium state. Cancer Res. 2012; 72:3987-96.
39. Finak G, Bertos N, Pepin F, Sadekova S, Souleimanova M, Zhao H, et al. Stromal gene expression predicts clinical outcome in breast cancer. Nat Med. 2008; 14:518-27.
40. Kristensen V N, Vaske C J, Ursini-Siegel J, Van Loo P, Nordgard S H, Sachidanandam R, et al. Integrated molecular profiles of invasive breast tumors and ductal carcinoma in situ (DCIS) reveal differential vascular and interleukin signaling. Proc Natl Acad Sci U S A. 2011.
41. Teschendorff A E, Menon U, Gentry-Maharaj A, Ramus S J, Gayther S A, Apostolidou S, et al. An epigenetic signature in peripheral blood predicts active ovarian cancer. PLoS One. 2009; 4:e8274.
42. Widschwendter M, Apostolidou S, Raum E, Rothenbacher D, Fiegl H, Menon U, et al. Epigenotyping in peripheral blood cell DNA and breast cancer risk: a proof of principle study. PLoS One. 2008; 3:e2656.
43. Xu Z, Bolick S C, DeRoo L A, Weinberg C R, Sandler D P, Taylor J A. Epigenome-wide association study of breast cancer using prospectively collected sister study samples. J Natl Cancer Inst. 2013; 105:694-700.
44. Koestler D C, Marsit C J, Christensen B C, Accomando W, Langevin S M, Houseman E A, et al. Peripheral blood immune cell methylation profiles are associated with nonhematopoietic cancers. Cancer Epidemiol Biomarkers Prey. 2012; 21:1293-302.
45. Langevin S M, Houseman E A, Accomando W P, Koestler D C, Christensen B C, Nelson H H, et al. Leukocyte-adjusted epigenome-wide association studies of blood from solid tumor patients. Epigenetics. 2014; 9:884-95.
46. Kanof M E, Smith P D, Zola H. PREPARATION OF HUMAN MONONUCLEAR CELL POPULATIONS AND SUBPOPULATIONS. Current Protocols in Immunology.
47. Morris T J, Butcher L M, Feber A, Teschendorff A E, Chakravarthy A R, Wojdacz T K, et al. ChAMP: 450k Chip Analysis Methylation Pipeline. Bioinformatics. 2014; 30:428-30.
48. Marzouka N A, Nordlund J, Backlin C L, Lonnerholm G, Syvanen A C, Carlsson Almlof J. CopyNumber450kCancer: baseline correction for accurate copy number calling from the 450k methylation array. Bioinformatics. 2015.
49. Houseman E A, Accomando W P, Koestler D C, Christensen B C, Marsit C J, Nelson H H, et al. DNA methylation arrays as surrogate measures of cell mixture distribution. BMC Bioinformatics. 2012; 13:86.
50. Smyth G K, Michaud J, Scott HS. Use of within-array replicate spots for assessing differential expression in microarray experiments. Bioinformatics. 2005; 21:2067-75.
51. Goeman J J. L1 penalized estimation in the Cox proportional hazards model. Biometrical journal Biometrische Zeitschrift. 2010; 52:70-84.
52. Wan E S, Qiu W, Carey V J, Morrow J, Bacherman H, Foreman M G, et al. Smoking Associated Site Specific Differential Methylation in Buccal Mucosa in the COPDGene Study. Am J Respir Cell Mol Biol. 2014.
53. Allione A, Marcon F, Fiorito G, Guarrera S, Siniscalchi E, Zijno A, et al. Novel Epigenetic Changes Unveiled by Monozygotic Twins Discordant for Smoking Habits. PLoS One. 2015;10:e0128265.
54. Cheng L, Liu J, Li B, Liu S, Li X, Tu H. Cigarette smoke-induced hypermethylation of the GCLC gene is associated with chronic obstructive pulmonary disease. Chest. 2015.
55. Li H, Hedmer M, Wojdacz T, Hossain M B, Lindh C H, Tinnerberg H, et al. Oxidative stress, telomere shortening, and DNA methylation in relation to low-to-moderate occupational exposure to welding fumes. Environ Mol Mutagen. 2015.
56. Liu J, Morgan M, Hutchison K, Calhoun V D. A study of the influence of sex on genome wide methylation. PLoS One.5:e10028.
57. Horvath S. DNA methylation age of human tissues and cell types. Genome Biol. 2013; 14:R115.
58. Stefanska B, Huang J, Bhattacharyya B, Suderman M, Hallett M, Han Z G, et al. Definition of the landscape of promoter DNA hypomethylation in liver cancer. Cancer Res. 2011.
59. Mandrekar J N. Receiver operating characteristic curve in diagnostic test assessment. J Thorac Oncol. 2010; 5:1315-6.
60. Di Bisceglie A M. Hepatitis B and hepatocellular carcinoma. Hepatology. 2009; 49:S56-60.
61. Hayashi P H, Di Bisceglie A M. The progression of hepatitis B- and C-infections to chronic liver disease and hepatocellular carcinoma: epidemiology and pathogenesis. Med Clin North Am. 2005; 89:371-89.

What is claimed is:

1. A kit for predicting hepatocellular carcinoma (HCC) stages and chronic hepatitis, comprising means and reagents to detect DNA methylation levels of a profile of DNA methylation signatures in peripheral blood mononuclear cells or T cell, wherein the DNA methylation levels correlate with the HCC stages and chronic hepatitis, wherein the profile of DNA methylation signatures consists of a combination of CG IDs listed below:

| | | | | | |
|---|---|---|---|---|---|
| cg05375333 | cg24304617 | cg08649216 | cg15775914 | cg06098530 | cg04536922 |
| cg23679141 | cg26009832 | cg06908855 | cg21585138 | cg15514380 | cg20838429 |
| cg01546046 | cg27090007 | cg11412036 | cg00744866 | cg19988492 | cg21542922 |
| cg10036013 | cg24958366 | cg23824801 | cg08306955 | cg00361155 | cg11356004 |
| cg12829666 | cg17479131 | cg27408285 | cg15009198 | cg05423018 | cg19140262 |
| cg15011899 | cg27644327 | cg01810593 | cg18878210 | cg13710613 | cg05033369 |
| cg02001279 | cg11031737 | cg19795616 | cg02717454 | cg07072643 | cg09048334 |
| cg15188939 | cg09800500 | cg27284331 | cg22344162 | cg04018625 | cg04385818 |
| cg23311108 | cg02313495 | cg08575688 | cg26923863 | cg01238991 | cg01214050 |
| cg09789584 | cg16324306 | cg05486191 | cg15447825 | cg17741339 | cg14361741 |
| cg22301128 | cg02914652 | cg04171808 | cg04771084 | cg18132851 | cg16292016 |
| cg11737318 | cg11057824 | cg14276584 | cg23981150 | cg02556954 | cg14783904 |
| cg07118376 | cg26407558 | cg03496780 | cg24383056 | cg01359822 | cg26250154 |
| cg13978347 | cg09451574 | cg14375111 | cg24232444 | cg22747380 | cg02758552 |
| cg23544996 | cg21156970 | cg08944236 | cg22281935 | cg00211609 | cg21811450 |
| cg16306870 | cg01732538 | cg02142483 | cg22110158 | cg11911769 | cg03432151 |
| cg03731740 | cg10312296 | cg23102014 | cg04398282 | cg15755348 | cg08455089 |
| cg02749789 | cg17704839 | cg25683268 | cg08946713 | cg25195795 | cg17766305 |
| cg08123444 | cg24742520 | cg20460227 | cg24056269 | cg06151145 | cg06349546 |
| cg15747825 | cg14983135 | cg17163729 | cg15118835 | cg00568910 | cg23017594 |
| cg23829949 | cg21164050 | cg01417062 | cg14189441 | cg15146122 | cg12813441 |
| cg16712679 | cg06879746 | cg13146484 | cg16111924 | cg13615971 | cg01411912 |
| cg12820627 | cg27057509 | cg18417954 | cg27089675 | cg06194421 | cg15374754 |
| cg17534034 | cg23857976 | cg13913085 | cg07128102 | cg01966878 | cg00093544 |
| cg05591270 | cg05228338 | cg12705693 | cg18556587 | cg16565409 | cg14711743 |
| cg13219008 | cg24783785 | cg21579239 | cg02863594 | cg03044573 | cg00483304 |
| cg15607708 | cg27457290 | cg10274682 | cg08577341 | cg10469659 | cg24376286 |
| cg22475353 | cg14199837 | cg19389852 | cg12306086 | cg16240816 | cg27638509 |
| cg27296330 | cg25104397 | cg01839860 | cg21700582 | cg21487856 | cg11300809 |
| cg24449629 | cg20592700 | cg20222519 | cg14774438 | cg23486701 | cg09244071 |
| cg12177922 | cg27010159 | cg02272851 | cg15123819 | cg24640156 | cg00014638 |
| cg23004466 | cg14898127 | cg14734614 | cg00759807 | cg05086021 | cg00697672 |
| cg01696603 | cg11783497 | cg27120934 | cg07929642 | cg03899643 | cg01116137 |
| cg03639671 | cg08861115 | cg10078703 | cg08134863 | cg11556164 | cg20250700 |
| cg10203922 | cg15966610 | cg05099186 | cg20228731 | cg25135755 | cg15867698 |
| cg13749822 | cg13299325 | cg11767757 | cg23493018 | cg08113187 | cg11151251 |
| cg12263794 | cg22547775 | cg09545443 | cg04071270 | cg27588356 | cg05577016 |
| cg23157190 | cg22945413 | cg20427318 | cg20750319 | cg01611777 | cg01933228 |
| cg21406217 | cg15046123 | cg01698579 | cg12050434 | cg12299554 | cg11006453 |
| cg08247053 | cg26405097 | cg12691488 | cg00458932 | cg14356440 | cg03555836 |
| cg26576206 | cg03483626 | cg08568561 | cg25708982 | cg18482303 | cg02482718 |
| cg07212747 | cg14531436 | cg13943141 | cg12592365 | cg15323084 | cg24065504 |
| cg22872033 | cg20587236 | cg13619522 | cg19780570 | cg22876402 | cg09340198 |
| cg27186013 | cg24284882 | cg05502766 | cg20187173 | cg17092349 | cg22143698 |
| cg19851487 | cg17226602 | cg06445016 | cg07772781 | cg02782634 | cg07065759 |
| cg03481488 | cg22707529 | cg10895875 | cg01828328 | cg09987993 | cg21751540 |
| cg12598524 | cg19945957 | cg08634082 | cg05725404 | cg26401541 | cg20956548 |
| cg10761639 | cg05460226 | cg20944521 | cg14426660 | cg00248242 | cg18731803 |
| cg00350932 | cg25364972 | cg03252499 | cg04998202 | cg09514545 | cg09639931 |
| cg14914552 | cg00754989 | cg14762436 | cg07381872 | cg16476382 | cg16810031 |
| cg07504763 | cg01994308 | cg19266387 | cg14193653 | cg00189276 | cg10861953 |
| cg25279586 | cg23837109 | cg17934470 | cg22675447 | cg08858441 | cg12628061 |
| cg12019814 | cg10892950 | cg00758915 | cg09479286 | cg20874210 | cg06874640 |
| cg05941376 | cg02976588 | cg27143049 | cg00426720 | cg00321614 | cg15006843 |
| cg23044884 | cg24576298 | cg23880736 | cg05999692 | cg08226047 | cg25522867 |
| cg15891076 | cg12344600 | cg04090347 | cg10784548 | cg02265379 | cg01124132 |
| cg07145988 | cg27544294 | cg22515654 | cg12201380 | cg19925215 | cg10536529 |
| cg09635768 | cg00448395 | cg03062944 | cg05961707 | cg10995381 | cg16517298 |
| cg18882449 and | cg03909800. | | | | |

2. The kit according to claim 1, wherein said CG IDs are derived from the DNA of peripheral blood mononuclear cells (PBMCs).

3. The kit according to claim 1, wherein said DNA methylation signature is derived using a genome wide DNA methylation mapping method.

4. The kit according to claim 3, wherein the DNA methylation mapping method is selected from the group consisting of Illumina 450K array, illumine 850K array, genome wide bisulfite sequencing, methylated DNA Immunoprecipitation (MeDIP) sequencing, and hybridization with oligonucleotide arrays.

5. The kit according to claim 1, further comprising a primer for STAP1 (CG ID: cg04398282), wherein said primer is selected from the group consisting of:
AGTYATGTYTTYTGYAAATAAAAATGGAYAYY
(SEQ ID NO: 16, outside forward),
TTRCTTTTTAACCACCAACACTACC
(SEQ ID NO: 17, outside reverse),
YYGTTTYTTTYATYTTYTGGTGATGTTAA
(SEQ ID NO: 18, nested forward),
ARARRRCAATCTCTRRRTAATCCACATRTR
(SEQ ID NO: 19, nested reverse),
and
GGTGATGTTAATYTTYTGTTTA
(SEQ ID NO: 20, sequencing primer).

6. The kit according to claim 1, wherein the profile of DNA methylation signatures for predicting HCC stages and chronic hepatitis is:
the profile of DNA methylation signatures for separating HCC stage 1 from controls consists of a combination of CG IDs selected from the group consisting of cg14983135, cg10203922, cg05941376, cg14762436, cg12019814, cg14426660, cg18882449, and cg02914652;
the profile of DNA methylation signatures for separating HCC stage 2 from controls consists of a combination of CG IDs selected from the group consisting of cg05941376, cg15188939, cg12344600, cg03496780, and cg12019814;
the profile of DNA methylation signatures for separating HCC stage 3 from controls consists of a combination of CG IDs selected from the group consisting of cg05941376, cg02782634, cg27284331, cg12019814, and cg23981150;
the profile of DNA methylation signatures for separating HCC stage 4 from controls consists of a combination of CG IDs selected from the group consisting of cg02782634, cg05941376, cg10203922, cg12019814, cg14914552, cg21164050, and cg23981150;
the profile of DNA methylation signatures for separating HCC stage 1 from hepatitis B consists of a combination of CG IDs selected from the group consisting of cg05941376, cg10203922, cg11767757, cg04398282, cg11151251, cg24742520, and cg14711743;
the profile of DNA methylation signatures for separating HCC stage 1 from stage 2-4 consists of a combination of CG IDs selected from the group consisting of cg03252499, cg03481488, cg04398282, cg10203922, cg11783497, cg13710613, cg14762436, and cg23486701;
the profile of DNA methylation signatures for separating HCC stage 2 from stage 3-4 consists of a combination of CG IDs selected from the group consisting of cg02914652, cg03252499, cg11783497, cg11911769, cg12019814, cg14711743, cg15607708, cg20956548, cg22876402, and cg24958366; and
the profile of DNA methylation signatures for separating HCC stage 1-3 from stage 4 consists of a combination of CG IDs selected from the group consisting of cg02782634, cg11151251, cg24958366, cg06874640, cg27284331, cg16476382, and cg14711743.

7. The kit according to claim 6, wherein said CG IDs are further grouped by using a statistical model.

8. The kit according to claim 7, wherein said statistical model comprises penalized regression or clustering analysis.
